(19) United States
(12) Patent Application Publication
(10) Pub. No.: US 2023/0405137 A1
(43) Pub. Date: Dec. 21, 2023

(54) Immunogenic compositions comprising conjugated capsular saccharide antigens and uses thereof

(71) Applicant: Pfizer Inc., New York, NY (US)

(72) Inventors: Paul Wayne Brown, Wildwood, MO (US); Kaushik Dutta, River Edge, NJ (US); Jason Arnold Lotvin, West Nyack, NY (US); Kelly Jeffrey Sackett, East Lyme, CT (US)

(21) Appl. No.: 18/252,124

(22) PCT Filed: Nov. 4, 2021
(86) PCT No.: PCT/IB2021/060217

§ 371 Date: May 8, 2023

Related U.S. Application Data
(60) Provisional application No. 63/111,765, filed on Nov. 10, 2020.

(51) Int. Cl. A61K 47/64; A61K 39/09; A61P 31/04

(52) U.S. Cl. CPC A61K 47/646; A61K 39/092; A61P 31/04; A61K 2039/6037

(57) ABSTRACT

The present invention relates to new immunogenic compositions comprising conjugated Streptococcus pneumoniae capsular saccharide antigens (glycoconjugates), kits comprising said immunogenic compositions and uses thereof. Immunogenic compositions of the present invention will typically comprise at least one glycoconjugate from a S. pneumoniae serotype not found in PREVNAR®, SYNFLORIX® and/or PREVNAR 13®. The invention also relates to vaccination of human subjects, in particular infants and elderly, against pneumococcal infections using said novel immunogenic compositions.

### FIELD OF THE INVENTION

The present invention relates to the field of immunogenic compositions and vaccines, their manufacture and the use of such compositions in medicine.

More particularly, it relates to isolated Streptococcus pneumoniae serotype 12F saccharide, glycoconjugates thereof, methods for making Streptococcus pneumoniae serotype 12F glycoconjugates and immunogenic composition comprising a Streptococcus pneumoniae serotype 12F glycoconjugate.

The invention also relates to analytical methods to analyze isolated S. pneumoniae serotype 12F polysaccharide, reduced serotype 12F polysaccharide or Streptococcus pneumoniae serotype 12F glycoconjugates.

The Streptococcus pneumoniae serotype 12F saccharide and glycoconjugates of the invention can be used as a vaccine.

### BACKGROUND OF THE INVENTION

Infections caused by pneumococci are a major cause of morbidity and mortality throughout the world. Pneumonia, febrile bacteraemia and meningitis are the most common manifestations of invasive pneumococcal disease, whereas bacterial spread within the respiratory tract may result in middle-ear infection, sinusitis or recurrent bronchitis. Compared with invasive disease, the non-invasive manifestations are usually less severe, but considerably more common.

In Europe and the United States, pneumococcal pneumonia is the most common community-acquired bacterial pneumonia, estimated to affect approximately 100 per 100,000 adults each year. The corresponding figures for febrile bacteraemia and meningitis are 15-19 per 100,000 and 1-2 per 100,000, respectively. The risk for one or more of these manifestations is much higher in infants and elderly people, as well as immune compromised persons of any age. Even in economically developed regions, invasive pneumococcal disease carries high mortality; for adults with pneumococcal pneumonia the mortality rate averages 10%-20%, while it may exceed 50% in the high-risk groups. Pneumonia is by far the most common cause of pneumococcal death worldwide.

The etiological agent of pneumococcal diseases, Streptococcus pneumoniae (pneumococcus), is a Gram-positive encapsulated coccus, surrounded by a polysaccharide capsule. Differences in the composition of this capsule permit serological differentiation between about 91 capsular types, some of which are frequently associated with pneumococcal disease, others rarely. Invasive pneumococcal infections include pneumonia, meningitis and febrile bacteremia; among the common non-invasive manifestations are otitis media, sinusitis and bronchitis.

T-independent antigens, for example saccharides, are antigens that elicit antibody production via B lymphocytes without involvement of T-cells. Conjugation of T-independent antigens to carrier proteins has been established as a way of enabling T-cell help to become part of the immune response for a normally T-independent antigen. Successful conjugate vaccines have been developed by conjugating bacterial capsular saccharides to carrier proteins; the carrier protein having the known effect of turning the T-independent saccharide antigen into a T-dependent antigen capable of triggering an immune memory response. Several carrier proteins are known in the art with tetanus toxoid, diphtheria toxoid, CRM₁₉₇ and protein D from Haemophilus influenzae being used as carrier protein in commercialised vaccines. Pneumococcal conjugate vaccines (PCVs) are pneumococcal vaccines used to protect against disease caused by S. pneumoniae (pneumococcus). There are currently three PCV vaccines available on the global market: PREVNAR® (PREVENAR® in some countries) (heptavalent vaccine), SYNFLORIX® (a decavalent vaccine) and PREVNAR 13® (PREVENAR 13® in some countries) (tridecavalent vaccine).

There remains a major need for effective vaccines against Streptococcus pneumoniae infection that can safely be produced in high quantities.

### SUMMARY OF THE INVENTION

To meet these and other needs, the present invention relates to isolated polysaccharide with the following repeating unit:

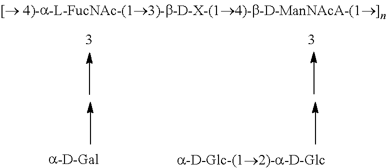

where n represents the number of repeating units and where X represents either N-acetylgalactosamine or 4-keto-N-acetyl-quinovosamine (2-acetamido-2,6-dideoxy-xylo-hexos-4-ulose).

In an aspect, the isolated polysaccharide comprises between about 99.9 to about 50 N-acetylgalactosamine residues and about 0.1 to about 50 4-keto-N-acetyl-quinovosamine residues in every 100 saccharide repeat units of the polysaccharide.

In an aspect the invention relates to an isolated S. pneumoniae serotype 12F capsular polysaccharide comprising between about 99.9 to about 50 N-acetylgalactosamine residues and about 0.1 to about 50 4-keto-N-acetyl-quinovosamine residues in every 100 saccharide repeat units of the polysaccharide.

The invention further relates to a S. pneumoniae serotype 12F glycoconjugate prepared by a process comprising the step of: a) reacting said isolated polysaccharide with an activating agent to produce an activated saccharide; and b) reacting the activated saccharide with a carrier protein.

In another aspect the invention relates to a S. pneumoniae serotype 12F glycoconjugate comprising a serotype 12F capsular polysaccharide comprising between about 0.05 to about 25 N-acetyl-D-fucosamine (D-FucNAc) residues in every 100 saccharide repeat units of the polysaccharide. In a particular aspect, the invention further relates to a S. pneumoniae serotype 12F glycoconjugate comprising a serotype 12F capsular polysaccharide comprising between about 0.05 to about 25 N-acetyl-D-fucosamine (D-FucNAc) residues and between about 0.05 to about 25 N-acetyl-D-quinovosamine (D-QuiNAc) in every 100 saccharide repeat units of the polysaccharide.

In an aspect, the glycoconjugates are prepared using reductive amination.

The invention further relates to immunogenic composition comprising the above polysaccharides or glycoconjugates as well as their use as a medicament, and in particular as a vaccine.

### DETAILED DESCRIPTION OF THE DRAWINGS

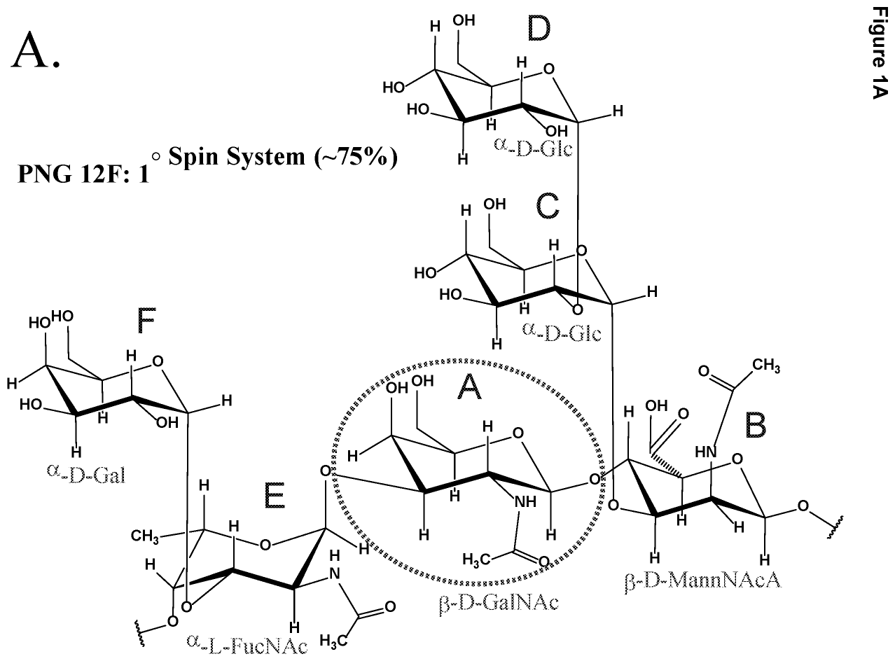

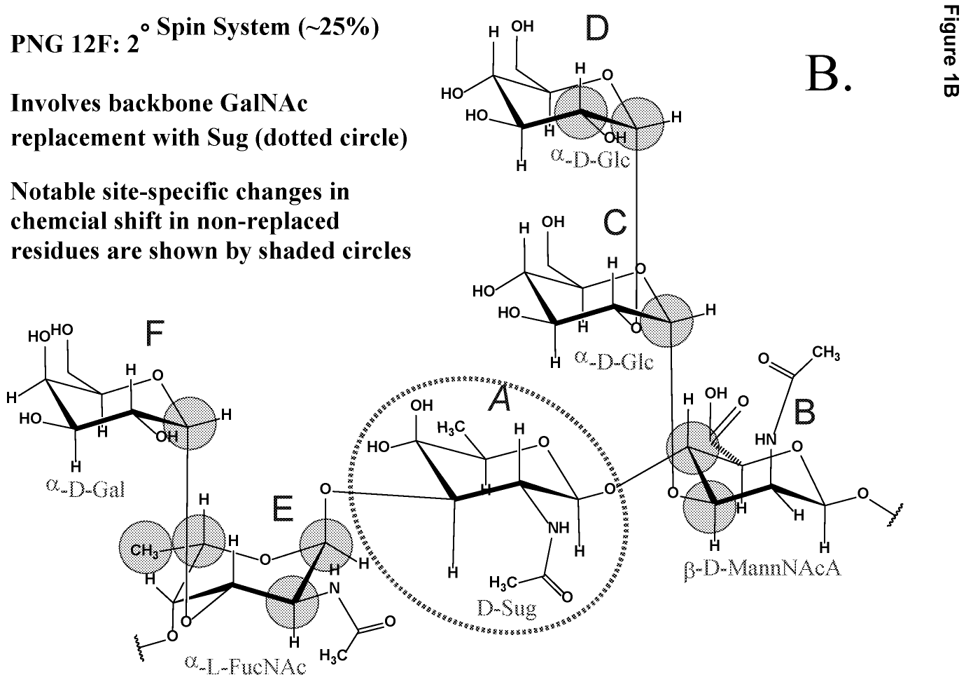

FIGS. 1A to 1C. Schematic of pneumococcal polysaccharide 12F repeat unit organization and populations, including the primary population (A) at ˜75-80 mol % consistent with Leontein et al. ((1981) Can. J. Chem. 59: 2081-2085), and secondary population (B) at ˜20-25 mol % characterized by replacement of GalNAc with Sug (keto-sugar). In the secondary spin system, the backbone and branched residue ¹³C and/or ¹H site-specific resonances significantly affected by Sug residue incorporation are shown in shaded circles. (C) Schematic representation of the average pneumococcal polysaccharide 12F repeat unit with the central backbone residue shown as either GalNAc or Sug based on statistical average (75%/25%).

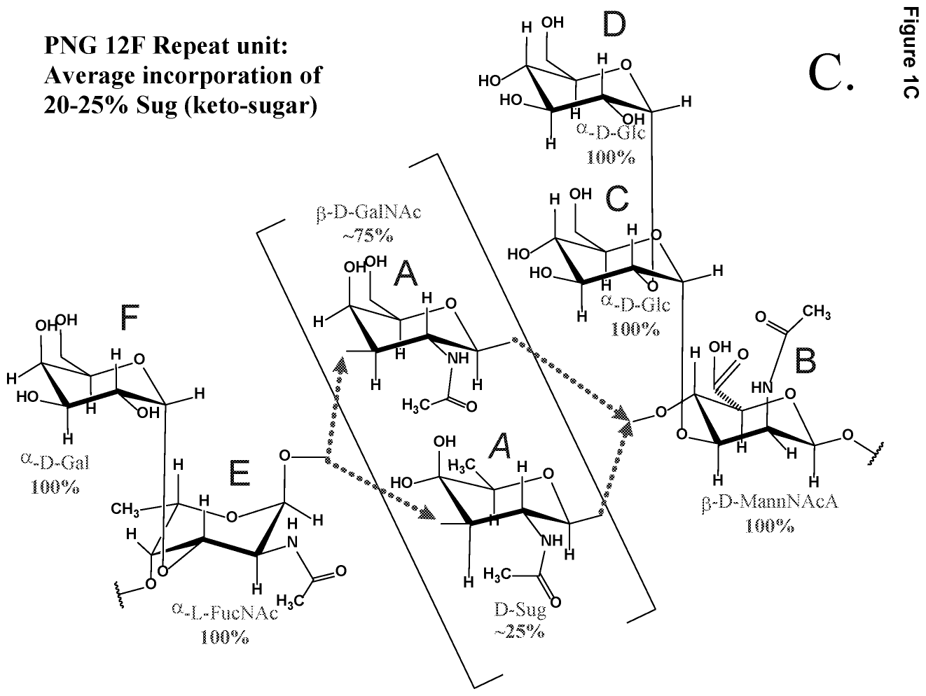

FIG. 2. ¹H and ¹³C chemical shift assignment for pneumococcal polysaccharide 12F Sug residue in hydrate form. Sug (also referred as 4-keto-N-acetyl-quinovosamine, 2-acetamido-2,6-dideoxy-D-xylo-4-hexulose or 4KQ in the present document) is incorporated in the backbone of the repeat unit by replacement of GalNAc residue.

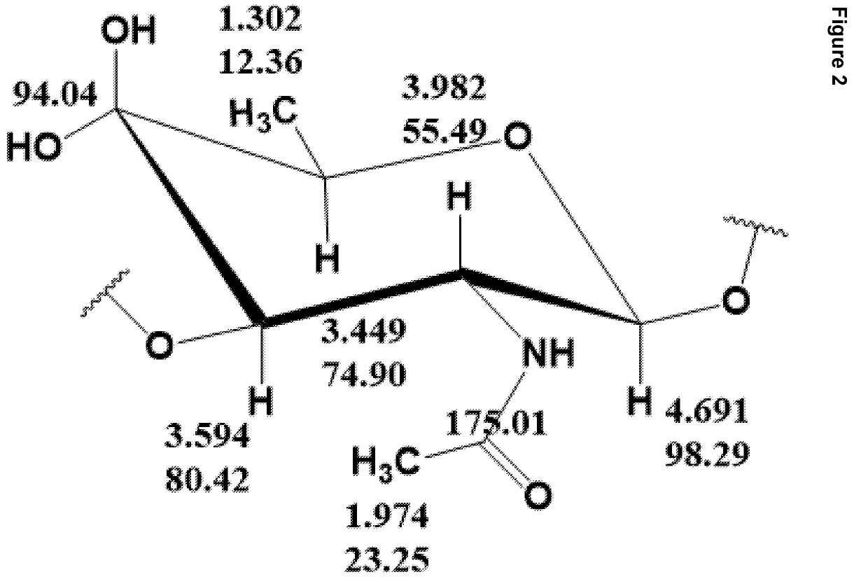

FIG. 3. Ketone/Hydrate Equilibrium

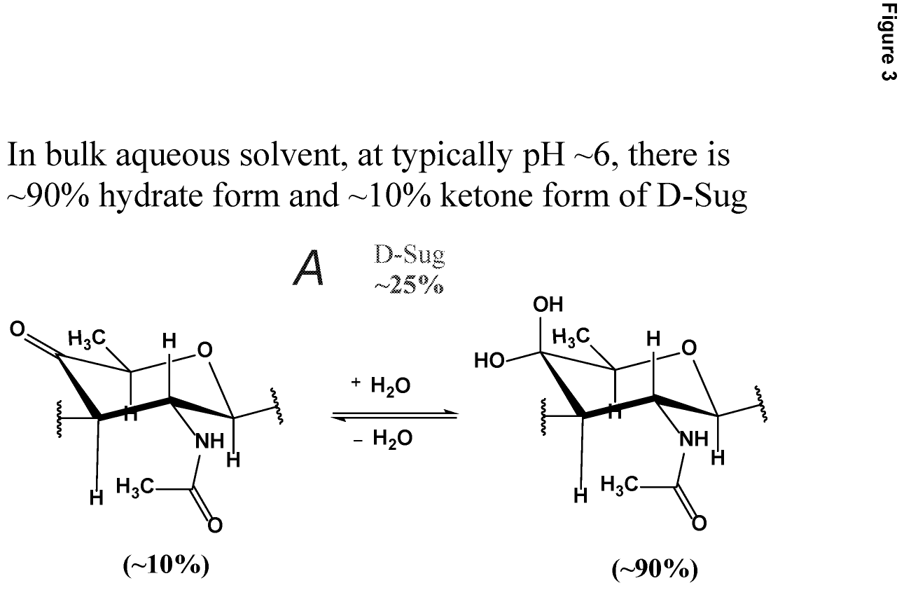

FIG. 4. Eleven NOESY correlations providing further support of D-Sug replacing GalNAc in the polysaccharide 12F repeat unit are shown.

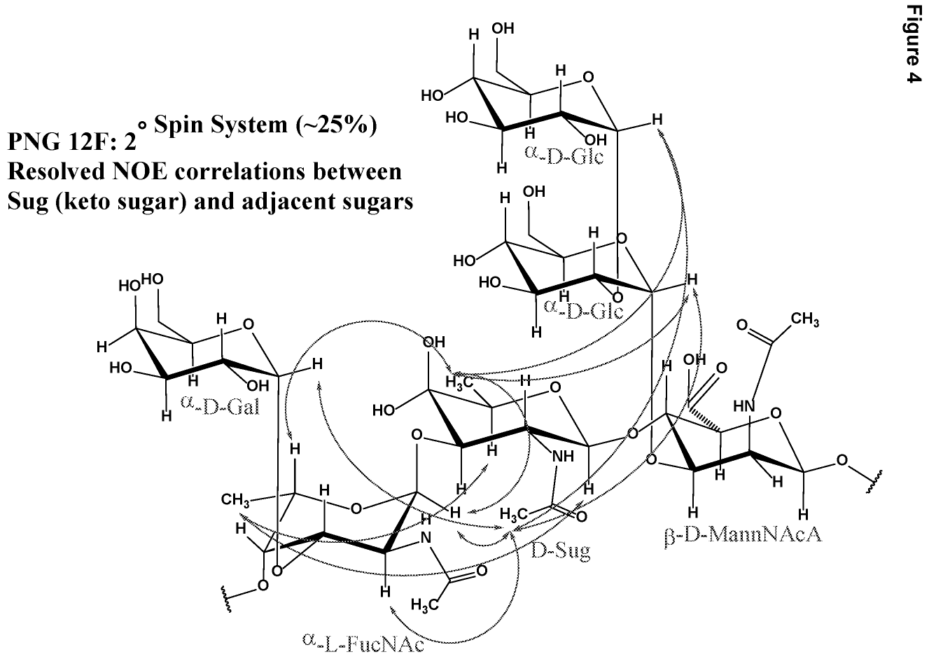

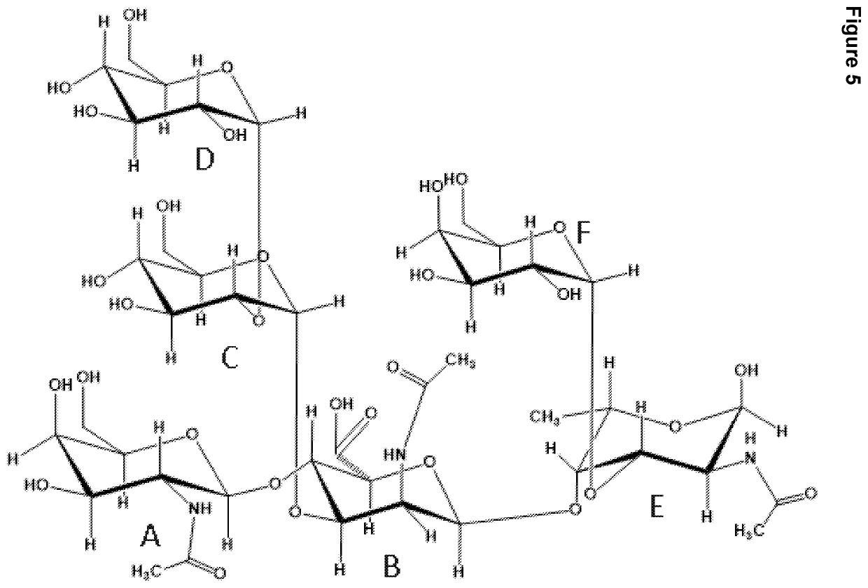

FIG. 5. The repeat unit containing GalNAc of the 12F polysaccharide as detected by In-source collision-induced dissociation (IS-CID)

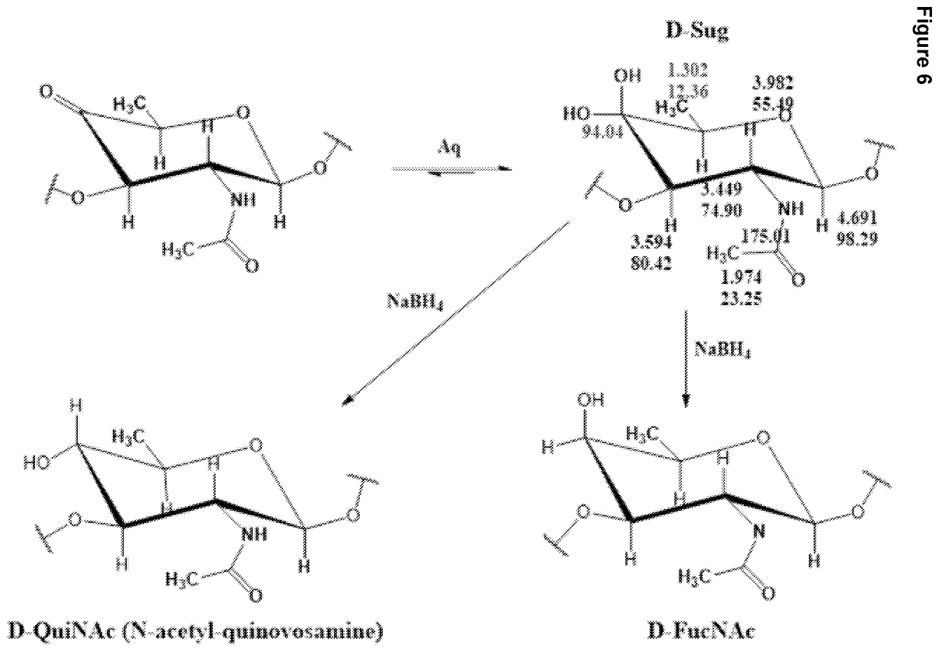

FIG. 6. Schematic of serotype 12F Sug residue (4-keto-N-acetyl-quinovosamine) showing ketone/hydrate equilibrium in aqueous solvent, as well as changes due to specific ketone reduction using NaBH₄.

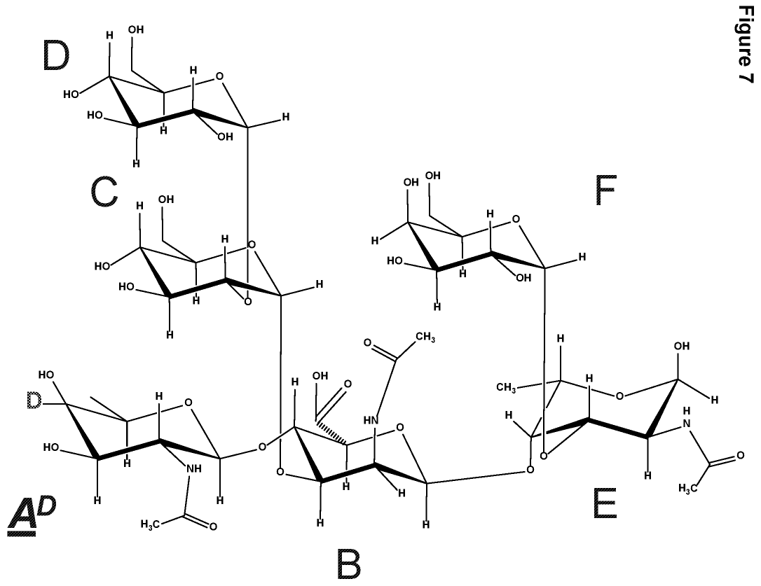

FIG. 7. Schematic of serotype 12F repeat unit containing Sug residue after reduction with NaBD₄.

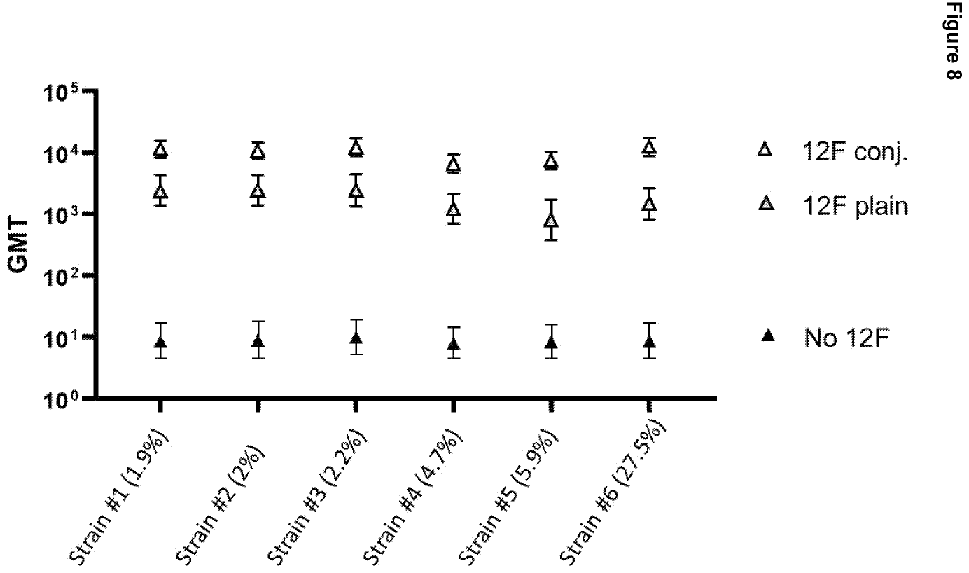

FIG. 8. Immune Sera from subjects immunized with a multi-valent vaccine containing a 12F conjugate (12F conj.), a multi-valent vaccine containing plain 12F (12F plain) or a multi-valent vaccine which did not contain 12F polysaccharide were tested regarding their ability to elicit bacterial killing responses of isolates with 4KQ modification levels between 1.9% to 27.5%.

### DETAILED DESCRIPTION OF THE INVENTION

The present invention is based, in part, on the identification of novel pneumococcal polysaccharide structure(s) by using NMR spectroscopy. It is believed that the structure provided herein is the first identification or the first correct identification of S. pneumoniae serotype 12F.

The S. pneumoniae serotype 12F polysaccharide was produced from different strains and purified. The produced (and purified) polysaccharide was used to generate polysaccharide-protein conjugate (glycoconjugates). S. pneumoniae serotype 12F has a unique polysaccharide structure, which results in a unique conjugate production process.

### 1. Isolated Streptococcus pneumoniae Serotype 12F Saccharide of the Invention

As used herein, the term “isolated” in connection with a polysaccharide refers to isolation of S. pneumoniae serotype specific capsular polysaccharide from purified polysaccharide using purification techniques known in the art, including the use of centrifugation, depth filtration, precipitation, ultrafiltration, treatment with activate carbon, diafiltration and/or column chromatography. Generally, an isolated polysaccharide refers to partial removal of proteins, nucleic acids and non-specific endogenous polysaccharide (C-polysaccharide). The isolated polysaccharide contains less than 10%, 8%, 6%, 4%, or 2% protein impurities and/or nucleic acids. The isolated polysaccharide contains less than 20% of C-polysaccharide with respect to type specific polysaccharides.

The structure of S. pneumoniae serotype 12F capsular polysaccharide has been previously published (Leontein et al. (1981) Can. J. Chem. 59: 2081-2085).

According to Leontein et al., the polysaccharide repeating unit of serotype 12F consists of a linear trisaccharide backbone (one N-acetylfucosamine (FucpNAc), one N-acetylgalactosamine (GalpNAc) and one N-acetylmannuronic acid (ManpNAcA)) with two branches: a pendant α-galactopyranose (Galp) linked at C3 of FucpNAc and an α-Glcp-(1→2)-α-Glcp disaccharide branch linked at C3 of ManpNAcA.

It has been surprisingly found by the inventors that the structure of S. pneumoniae serotype 12F is different. For the first time the inventors found that the serotype 12F polysaccharide actually contains partial substitution of N-acetyl-galactosamine by 4-keto-N-acetyl-quinovosamine (also referred as Sug, D-Sug, 2-acetamido-2,6-dideoxy-D-xylo-4-hexulose or 4KQ in the present document))

Presence of 4KQ incorporation was examined in different clinical serotype 12F isolates and different serotype 12F strains. All clinical isolates studied had some level of 4KQ incorporation, indicating that 4KQ substitution is common in clinical serotype 12F isolates.

Accordingly, in one embodiment, the present invention provides an isolated polysaccharide with the following repeating unit:

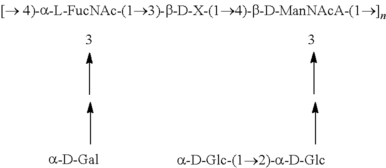

where n represents the number of repeating units and where X represents either N-acetylgalactosamine or 4-keto-N-acetyl-quinovosamine.

In one embodiment, the present invention provides an isolated polysaccharide with the following repeating unit:

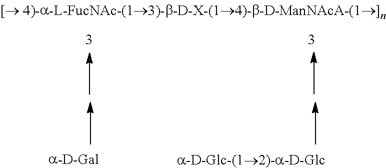

where n represents the number of repeating units, where X represents either N-acetylgalactosamine or 4-keto-N-acetyl-quinovosamine, where the polysaccharide comprises between about 99.9 to about 50 N-acetylgalactosamine residues and about 0.1 to about 50 4-keto-N-acetyl-quinovosamine residues in every 100 saccharide repeat units of the polysaccharide.

In certain embodiments, the isolated polysaccharide comprises between about 99.1 to about 50 N-acetylgalactosamine residues and about 0.9 to about 50 4-keto-N-acetyl-quinovosamine residues in every 100 saccharide repeat units of the polysaccharide.

In certain embodiments, the isolated polysaccharide comprises between about 99 to about 50 N-acetylgalactosamine residues and about 1 to about 50 4-keto-N-acetyl-quinovosamine residues in every 100 saccharide repeat units of the polysaccharide.

In certain embodiments, the isolated polysaccharide comprises between about 98 to about 50 N-acetylgalactosamine residues and about 2 to about 50 4-keto-N-acetyl-quinovosamine residues in every 100 saccharide repeat units of the polysaccharide. In certain embodiments, the isolated polysaccharide comprises between about 97 to about 50 N-acetylgalactosamine residues and about 3 to about 50 4-keto-N-acetyl-quinovosamine residues in every 100 saccharide repeat units of the polysaccharide. In certain embodiments, the isolated polysaccharide comprises between about 96 to about 50 N-acetylgalactosamine residues and about 4 to about 50 4-keto-N-acetyl-quinovosamine residues in every 100 saccharide repeat units of the polysaccharide. In certain embodiments, the isolated polysaccharide comprises between about 95 to about 50 N-acetylgalactosamine residues and about 5 to about 50 4-keto-N-acetyl-quinovosamine residues in every 100 saccharide repeat units of the polysaccharide. In certain embodiments, the isolated polysaccharide comprises between about 94 to about 50 N-acetylgalactosamine residues and about 6 to about 50 4-keto-N-acetyl-quinovosamine residues in every 100 saccharide repeat units of the polysaccharide.

In certain embodiments, the isolated polysaccharide comprises between about 93 to about 50 N-acetylgalactosamine residues and about 7 to about 50 4-keto-N-acetyl-quinovosamine residues in every 100 saccharide repeat units of the polysaccharide. In certain embodiments, the isolated polysaccharide comprises between about 92 to about 50 N-acetylgalactosamine residues and about 8 to about 50 4-keto-N-acetyl-quinovosamine residues in every 100 saccharide repeat units of the polysaccharide. In certain embodiments, the isolated polysaccharide comprises between about 91 to about 50 N-acetylgalactosamine residues and about 9 to about 50 4-keto-N-acetyl-quinovosamine residues in every 100 saccharide repeat units of the polysaccharide. In certain embodiments, the isolated polysaccharide comprises between about 90 to about 50 N-acetylgalactosamine residues and about 10 to about 50 4-keto-N-acetyl-quinovosamine residues in every 100 saccharide repeat units of the polysaccharide.

In certain embodiments, the isolated polysaccharide comprises between about 99.1 to about 55 N-acetylgalactosamine residues and about 0.9 to about 45 4-keto-N-acetyl-quinovosamine residues in every 100 saccharide repeat units of the polysaccharide.

In certain embodiments, the isolated polysaccharide comprises between about 99 to about 55 N-acetylgalactosamine residues and about 1 to about 45 4-keto-N-acetyl-quinovosamine residues in every 100 saccharide repeat units of the polysaccharide.

In certain embodiments, the isolated polysaccharide comprises between about 98 to about 55 N-acetylgalactosamine residues and about 2 to about 45 4-keto-N-acetyl-quinovosamine residues in every 100 saccharide repeat units of the polysaccharide. In certain embodiments, the isolated polysaccharide comprises between about 97 to about 55 N-acetylgalactosamine residues and about 3 to about 45 4-keto-N-acetyl-quinovosamine residues in every 100 saccharide repeat units of the polysaccharide. In certain embodiments, the isolated polysaccharide comprises between about 96 to about 55 N-acetylgalactosamine residues and about 4 to about 45 4-keto-N-acetyl-quinovosamine residues in every 100 saccharide repeat units of the polysaccharide. In certain embodiments, the isolated polysaccharide comprises between about 95 to about 55 N-acetylgalactosamine residues and about 5 to about 45 4-keto-N-acetyl-quinovosamine residues in every 100 saccharide repeat units of the polysaccharide. In certain embodiments, the isolated polysaccharide comprises between about 94 to about 55 N-acetylgalactosamine residues and about 4 to about 45 4-keto-N-acetyl-quinovosamine residues in every 100 saccharide repeat units of the polysaccharide. In certain embodiments, the isolated polysaccharide comprises between about 93 to about 55 N-acetylgalactosamine residues and about 7 to about 45 4-keto-N-acetyl-quinovosamine residues in every 100 saccharide repeat units of the polysaccharide. In certain embodiments, the isolated polysaccharide comprises between about 92 to about 55 N-acetylgalactosamine residues and about 8 to about 45 4-keto-N-acetyl-quinovosamine residues in every 100 saccharide repeat units of the polysaccharide. In certain embodiments, the isolated polysaccharide comprises between about 91 to about 55 N-acetylgalactosamine residues and about 9 to about 45 4-keto-N-acetyl-quinovosamine residues in every 100 saccharide repeat units of the polysaccharide. In certain embodiments, the isolated polysaccharide comprises between about 90 to about 55 N-acetylgalactosamine residues and about 10 to about 45 4-keto-N-acetyl-quinovosamine residues in every 100 saccharide repeat units of the polysaccharide.

In certain embodiments, the isolated polysaccharide comprises between about 99.9 to about 75 N-acetylgalactosamine residues and about 0.1 to about 25 4-keto-N-acetyl-quinovosamine residues in every 100 saccharide repeat units of the polysaccharide.

In certain embodiments, the isolated polysaccharide comprises between about 99.1 to about 75 N-acetylgalactosamine residues and about 0.9 to about 25 4-keto-N-acetyl-quinovosamine residues in every 100 saccharide repeat units of the polysaccharide.

In certain embodiments, the isolated polysaccharide comprises between about 99 to about 75 N-acetylgalactosamine residues and about 1 to about 25 4-keto-N-acetyl-quinovosamine residues in every 100 saccharide repeat units of the polysaccharide.

In certain embodiments, the isolated polysaccharide comprises between about 98 to about 75 N-acetylgalactosamine residues and about 2 to about 25 4-keto-N-acetyl-quinovosamine residues in every 100 saccharide repeat units of the polysaccharide. In certain embodiments, the isolated polysaccharide comprises between about 97 to about 75 N-acetylgalactosamine residues and about 3 to about 25 4-keto-N-acetyl-quinovosamine residues in every 100 saccharide repeat units of the polysaccharide. In certain embodiments, the isolated polysaccharide comprises between about 96 to about 75 N-acetylgalactosamine residues and about 4 to about 25 4-keto-N-acetyl-quinovosamine residues in every 100 saccharide repeat units of the polysaccharide. In certain embodiments, the isolated polysaccharide comprises between about 95 to about 75 N-acetylgalactosamine residues and about 5 to about 25 4-keto-N-acetyl-quinovosamine residues in every 100 saccharide repeat units of the polysaccharide. In certain embodiments, the isolated polysaccharide comprises between about 94 to about 75 N-acetylgalactosamine residues and about 4 to about 25 4-keto-N-acetyl-quinovosamine residues in every 100 saccharide repeat units of the polysaccharide. In certain embodiments, the isolated polysaccharide comprises between about 93 to about 75 N-acetylgalactosamine residues and about 7 to about 25 4-keto-N-acetyl-quinovosamine residues in every 100 saccharide repeat units of the polysaccharide. In certain embodiments, the isolated polysaccharide comprises between about 92 to about 75 N-acetylgalactosamine residues and about 8 to about 25 4-keto-N-acetyl-quinovosamine residues in every 100 saccharide repeat units of the polysaccharide. In certain embodiments, the isolated polysaccharide comprises between about 91 to about 75 N-acetylgalactosamine residues and about 9 to about 25 4-keto-N-acetyl-quinovosamine residues in every 100 saccharide repeat units of the polysaccharide. In certain embodiments, the isolated polysaccharide comprises between about 90 to about 75 N-acetylgalactosamine residues and about 10 to about 25 4-keto-N-acetyl-quinovosamine residues in every 100 saccharide repeat units of the polysaccharide.

In certain embodiments, the isolated polysaccharide comprises about 99.9 N-acetylgalactosamine residues and about 0.1 4-keto-N-acetyl-quinovosamine residues in every 100 saccharide repeat units of the polysaccharide.

In certain embodiments, the isolated polysaccharide comprises about 99.8 N-acetylgalactosamine residues and about 0.2 4-keto-N-acetyl-quinovosamine residues in every 100 saccharide repeat units of the polysaccharide.

In certain embodiments, the isolated polysaccharide comprises about 99.5 N-acetylgalactosamine residues and about 0.5 4-keto-N-acetyl-quinovosamine residues in every 100 saccharide repeat units of the polysaccharide.

In certain embodiments, the isolated polysaccharide comprises about 99 N-acetylgalactosamine residues and about 1 4-keto-N-acetyl-quinovosamine residues in every 100 saccharide repeat units of the polysaccharide.

In certain embodiments, the isolated polysaccharide comprises about 98 N-acetylgalactosamine residues and about 2 4-keto-N-acetyl-quinovosamine residues in every 100 saccharide repeat units of the polysaccharide.

In certain embodiments, the isolated polysaccharide comprises about 97 N-acetylgalactosamine residues and about 3 4-keto-N-acetyl-quinovosamine residues in every 100 saccharide repeat units of the polysaccharide.

In certain embodiments, the isolated polysaccharide comprises about 96 N-acetylgalactosamine residues and about 4 4-keto-N-acetyl-quinovosamine residues in every 100 saccharide repeat units of the polysaccharide.

In certain embodiments, the isolated polysaccharide comprises about 95 N-acetylgalactosamine residues and about 5 4-keto-N-acetyl-quinovosamine residues in every 100 saccharide repeat units of the polysaccharide.

In certain embodiments, the isolated polysaccharide comprises about 94 N-acetylgalactosamine residues and about 6 4-keto-N-acetyl-quinovosamine residues in every 100 saccharide repeat units of the polysaccharide.

In certain embodiments, the isolated polysaccharide comprises about 93 N-acetylgalactosamine residues and about 7 4-keto-N-acetyl-quinovosamine residues in every 100 saccharide repeat units of the polysaccharide.

In certain embodiments, the isolated polysaccharide comprises about 92 N-acetylgalactosamine residues and about 8 4-keto-N-acetyl-quinovosamine residues in every 100 saccharide repeat units of the polysaccharide.

In certain embodiments, the isolated polysaccharide comprises about 91 N-acetylgalactosamine residues and about 9 4-keto-N-acetyl-quinovosamine residues in every 100 saccharide repeat units of the polysaccharide.

In certain embodiments, the isolated polysaccharide comprises about 90 N-acetylgalactosamine residues and about 10 4-keto-N-acetyl-quinovosamine residues in every 100 saccharide repeat units of the polysaccharide.

In certain embodiments, the isolated polysaccharide comprises about 85 N-acetylgalactosamine residues and about 15 4-keto-N-acetyl-quinovosamine residues in every 100 saccharide repeat units of the polysaccharide.

In certain embodiments, the isolated polysaccharide comprises about 80 N-acetylgalactosamine residues and about 20 4-keto-N-acetyl-quinovosamine residues in every 100 saccharide repeat units of the polysaccharide.

In certain embodiments, the isolated polysaccharide comprises about 75 N-acetylgalactosamine residues and about 25 4-keto-N-acetyl-quinovosamine residues in every 100 saccharide repeat units of the polysaccharide.

In certain embodiments, the isolated polysaccharide comprises about 70 N-acetylgalactosamine residues and about 30 4-keto-N-acetyl-quinovosamine residues in every 100 saccharide repeat units of the polysaccharide.

In certain embodiments, the isolated polysaccharide comprises about 65 N-acetylgalactosamine residues and about 35 4-keto-N-acetyl-quinovosamine residues in every 100 saccharide repeat units of the polysaccharide.

In certain embodiments, the isolated polysaccharide comprises about 60 N-acetylgalactosamine residues and about 40 4-keto-N-acetyl-quinovosamine residues in every 100 saccharide repeat units of the polysaccharide.

In certain embodiments, the isolated polysaccharide comprises about 55 N-acetylgalactosamine residues and about 45 4-keto-N-acetyl-quinovosamine residues in every 100 saccharide repeat units of the polysaccharide.

In certain embodiments, the isolated polysaccharide comprises about 50 N-acetylgalactosamine residues and about 50 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.9 to about 50 N-acetylgalactosamine residues and about 0.1 to about 50 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.9 to about 55 N-acetylgalactosamine residues and about 0.1 to about 45 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.9 to about 75 N-acetylgalactosamine residues and about 0.1 to about 25 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99 to about 75 N-acetylgalactosamine residues and about 1 to about 25 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 95 to about 50 N-acetylgalactosamine residues and about 5 to about 50 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 95 to about 55 N-acetylgalactosamine residues and about 5 to about 45 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 95 to about 75 N-acetylgalactosamine residues and about 5 to about 25 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 90 to about 50 N-acetylgalactosamine residues and about 10 to about 50 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 90 to about 55 N-acetylgalactosamine residues and about 10 to about 45 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 90 to about 75 N-acetylgalactosamine residues and about 10 to about 25 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.9 to about 99.5 N-acetylgalactosamine residues and about 0.1 to about 0.5 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.9 to about 99 N-acetylgalactosamine residues and about 0.1 to about 1 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.9 to about 98 N-acetylgalactosamine residues and about 0.1 to about 2 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.9 to about 97 N-acetylgalactosamine residues and about 0.1 to about 3 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.9 to about 95 N-acetylgalactosamine residues and about 0.1 to about 5 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.8 to about 99.5 N-acetylgalactosamine residues and about 0.2 to about 0.5 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.8 to about 99 N-acetylgalactosamine residues and about 0.2 to about 1 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.8 to about 98 N-acetylgalactosamine residues and about 0.2 to about 2 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.8 to about 97 N-acetylgalactosamine residues and about 0.2 to about 3 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.8 to about 95 N-acetylgalactosamine residues and about 0.2 to about 5 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.5 to about 99 N-acetylgalactosamine residues and about 0.5 to about 1 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.5 to about 98 N-acetylgalactosamine residues and about 0.5 to about 2 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.5 to about 97 N-acetylgalactosamine residues and about 0.5 to about 3 4-keto-N-acetyl-quinovosamine residues in every 100 saccharide repeat units of the polysaccharide.

In one embodiment, the present invention provides an isolated S. pneumoniae serotype 12F capsular polysaccharide comprising between about 99.5 to about 95 N-acetylgalactosamine residues and about 0.5 to about 5 4-keto-N-acetyl-quinovosamine residues in every 100 saccharide repeat units of the polysaccharide.

In certain embodiments, the isolated polysaccharide has between 10 and 5,000 repeating units. In certain aspects, the isolated polysaccharide has between 50 and 4,500 repeating units. In certain aspects, the isolated polysaccharide has between 100 and 4,500 repeating units. In certain aspects, the isolated polysaccharide has between 150 and 2,000 repeating units.

Isolated capsular saccharides from S. pneumoniae serotype 12F are prepared by standard techniques known to those of ordinary skill in the art. Typically capsular polysaccharides are produced by growing a S. pneumoniae serotype 12F strain in a medium (e.g., in a soy-based medium), the polysaccharides are then prepared from the bacteria culture. Serotype 12F Streptococcus pneumoniae strains may be obtained from established culture collections (such as for example the Streptococcal Reference Laboratory (Centers for Disease Control and Prevention, Atlanta, GA)) or clinical specimens.

The population of the organism (S. pneumoniae serotype 12F) is often scaled up from a seed vial to seed bottles and passaged through one or more seed fermentors of increasing volume until production scale fermentation volumes are reached. At the end of the growth cycle the cells are lysed and the lysate broth is then harvested for downstream (purification) processing (see for example WO 2006/110381 and WO 2008/118752, U.S. Patent App. Pub. Nos. 2006/0228380, 2006/0228381, 2008/0102498 and US2008/0286838). The polysaccharides are typically purified through centrifugation, precipitation, ultra-filtration, and/or column chromatography (see for example WO 2006/110352, WO 2008/118752 and WO2020/170190).

The isolated polysaccharide can be characterized by different parameters including, for example the weight average molecular weight (Mw).

The molecular weight of the polysaccharide can be measured by Size Exclusion Chromatography (SEC) combined with Multiangle Laser Light Scattering detector (MALLS). In an embodiment, the isolated polysaccharide has a weight average molecular weight between 5 kDa and 5000 kDa. In an embodiment, the isolated polysaccharide has a weight average molecular weight between 5 kDa and 2000 kDa. In an embodiment, the isolated polysaccharide has a weight average molecular weight between 5 kDa and 1000 kDa. In an embodiment, the isolated polysaccharide has a weight average molecular weight between 5 kDa and 500 kDa. In an embodiment, the isolated capsular polysaccharide has a weight average molecular weight between 5 kDa and 400 kDa. In an embodiment, the isolated polysaccharide has a weight average molecular weight between 5 kDa and 300 kDa. In an embodiment, the isolated polysaccharide has a weight average molecular weight between 5 kDa and 200 kDa. In an embodiment, the isolated polysaccharide has a weight average molecular weight between 5 kDa and 100 kDa.

In an embodiment, the isolated polysaccharide has a weight average molecular weight between 50 kDa and 5000 kDa. In an embodiment, the isolated polysaccharide has a weight average molecular weight between 50 kDa and 2000 kDa. In an embodiment, the isolated polysaccharide has a weight average molecular weight between 50 kDa and 1000 kDa. In an embodiment, the isolated polysaccharide has a weight average molecular weight between 50 kDa and 500 kDa. In an embodiment, the isolated capsular polysaccharide has a weight average molecular weight between 50 kDa and 400 kDa. In an embodiment, the isolated polysaccharide has a weight average molecular weight between 50 kDa and 300 kDa. In an embodiment, the isolated polysaccharide has a weight average molecular weight between 50 kDa and 200 kDa. In an embodiment, the isolated polysaccharide has a weight average molecular weight between 50 kDa and 100 kDa.

In an embodiment, the isolated polysaccharide has a weight average molecular weight between 100 kDa and 5000 kDa. In an embodiment, the isolated polysaccharide has a weight average molecular weight between 100 kDa and 2000 kDa. In an embodiment, the isolated polysaccharide has a weight average molecular weight between 100 kDa and 1000 kDa. In an embodiment, the isolated polysaccharide has a weight average molecular weight between 100 kDa and 500 kDa. In an embodiment, the isolated polysaccharide has a weight average molecular weight between 100 kDa and 400 kDa. In an embodiment, the isolated polysaccharide has a weight average molecular weight between 100 kDa and 300 kDa. In an embodiment, the isolated polysaccharide has a weight average molecular weight between 100 kDa and 200 kDa.

In an embodiment, the isolated polysaccharide has a weight average molecular weight between 200 kDa and 5000 kDa. In an embodiment, the isolated polysaccharide has a weight average molecular weight between 200 kDa and 2000 kDa. In an embodiment, the isolated polysaccharide has a weight average molecular weight between 200 kDa and 1000 kDa. In an embodiment, the isolated polysaccharide has a weight average molecular weight between 200 kDa and 500 kDa. In an embodiment, the isolated polysaccharide has a weight average molecular weight between 200 kDa and 400 kDa. In an embodiment, the isolated polysaccharide has a weight average molecular weight between 200 kDa and 300 kDa.

In an embodiment, the isolated polysaccharide has a weight average molecular weight between 300 kDa and 5000 kDa. In an embodiment, the isolated polysaccharide has a weight average molecular weight between 300 kDa and 2000 kDa. In an embodiment, the isolated polysaccharide has a weight average molecular weight between 300 kDa and 1000 kDa. In an embodiment, the isolated polysaccharide has a weight average molecular weight between 300 kDa and 500 kDa. In an embodiment, the isolated polysaccharide has a weight average molecular weight between 300 kDa and 400 kDa.

In an embodiment, the isolated polysaccharide has a weight average molecular weight between 500 kDa and 5000 kDa. In an embodiment, the isolated polysaccharide has a weight average molecular weight between 500 kDa and 2000 kDa. In an embodiment, the isolated polysaccharide has a weight average molecular weight between 500 kDa and 1000 kDa. In a preferred embodiment, the isolated polysaccharide has a weight average molecular weight between 100 kDa and 500 kDa.

### 2. Streptococcus pneumoniae Serotype 12F Glycoconjugates of the Invention

The isolated polysaccharide described above may be activated (e.g., chemically activated) to make them capable of reacting (e.g. with a linker or directly with the carrier protein) and then incorporated into glycoconjugates, as further described herein.

For the purposes of the invention the term ‘glycoconjugate’ indicates a saccharide covalently linked to a carrier protein. In one embodiment a saccharide is linked directly to a carrier protein. In a second embodiment a saccharide is linked to a carrier protein through a spacer/linker.

In general, covalent conjugation of saccharides to carriers enhances the immunogenicity of saccharides as it converts them from T-independent antigens to T-dependent antigens, thus allowing priming for immunological memory. Conjugation is particularly useful for pediatric vaccines.

Before activation, the size of the isolated polysaccharide can be reduced while preserving critical features of the structure of the polysaccharide. Mechanical or chemical sizing maybe employed. In an embodiment, the size of the isolated polysaccharide is reduced by chemical hydrolysis. The size of the isolated polysaccharide can also be reduced by mechanical homogenization. In an embodiment, the size of the isolated polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

In an embodiment, the invention relates to a serotype 12F glycoconjugate prepared by a process comprising the step of: a) reacting an isolated polysaccharide of section 1 above with an activating agent to produce an activated saccharide; and b) reacting the activated saccharide with a carrier protein.

In an embodiment, the serotype 12F glycoconjugate of the present invention comprises a serotype 12F polysaccharide wherein the weight average molecular weight (Mw) of said polysaccharide before conjugation is between 50 kDa and 1,000 kDa.

In an embodiment, the serotype 12F glycoconjugate of the present invention comprises a serotype 12F polysaccharide wherein the weight average molecular weight (Mw) of said polysaccharide before conjugation is between 100 kDa and 600 kDa.

In an embodiment, the serotype 12F glycoconjugate of the present invention comprises a serotype 12F polysaccharide wherein the weight average molecular weight (Mw) of said polysaccharide before conjugation is between 150 kDa and 400 kDa.

In an embodiment, the serotype 12F glycoconjugate of the present invention comprises a serotype 12F polysaccharide wherein the weight average molecular weight (Mw) of said polysaccharide before conjugation is between 150 kDa and 300 kDa.

In some embodiments, the serotype 12F glycoconjugate of the invention has a weight average molecular weight (Mw) of between 250 kDa and 15,000 kDa.

In other embodiments, the serotype 12F glycoconjugate has a weight average molecular weight (Mw) of between 500 kDa and 2,500 kDa.

In still other embodiments, the serotype 12F glycoconjugate has a weight average molecular weight (Mw) of between 750 kDa and 2,500 kDa.

In preferred embodiments, the serotype 12F glycoconjugate has a weight average molecular weight (Mw) of between 1,000 kDa and 2,500 kDa.

Another way to characterize the serotype 12F glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the serotype 12F glycoconjugate of the invention is between 2 and 15. In an embodiment, the degree of conjugation of the serotype 12F glycoconjugate of the invention is between 2 and 10. In an embodiment, the degree of conjugation of the serotype 12F glycoconjugate of the invention is between 3 and 5. In an embodiment, the degree of conjugation of the serotype 12F glycoconjugate of the invention is between 2 and 6. In an embodiment, the degree of conjugation of the serotype 12F glycoconjugate of the invention is between 4 and 10.

The serotype 12F glycoconjugates of the invention may also be characterized by the ratio (weight/weight) of saccharide to carrier protein. In some embodiments, the ratio of serotype 12F polysaccharide to carrier protein in the glycoconjugate (w/w) is between 0.5 and 3.0. In other embodiments, the saccharide to carrier protein ratio (w/w) is between 0.5 and 2.0. In other embodiments, the saccharide to carrier protein ratio (w/w) is between 0.5 and 1.5. In other embodiments, the saccharide to carrier protein ratio (w/w) is between 0.8 and 1.2. In other embodiments, the saccharide to carrier protein ratio (w/w) is between 0.5 and 1.0. In other embodiments, the saccharide to carrier protein ratio (w/w) is between 1.0 and 1.5. In other embodiments, the saccharide to carrier protein ratio (w/w) is between 1.0 and 2.0. In further embodiments, the saccharide to carrier protein ratio (w/w) is between 0.8 and 1.2. In a preferred embodiment, the ratio of serotype 12F capsular polysaccharide to carrier protein in the conjugate is between 0.9 and 1.1. In some such embodiments, the carrier protein is CRM₁₉₇.

The process to prepare the serotype 12F glycoconjugate of the invention may comprise the use of reducing agent. In particular, unreacted aldehyde groups following oxidation (in particular when reductive amination is used, see below) may be capped using a suitable capping agent (reducing agent). In one embodiment this capping agent is sodium borohydride (NaBH₄).

As shown at Example 2, the 4KQ (4-keto-N-acetyl-quinovosamine) residue is sensitive to reduction using NaBH₄. Treatment of serotype 12F polysaccharide with NaBH₄ specifically reduces the position 4 of 4KQ residue from a ketone/hydrate to an alcohol and transform the residue 4KQ to a mixture of D-FucNAc and D-QuiNAc, characterized by position 4 hydroxyl at axial and equatorial orientations, respectively as illustrated in FIG. 6.

Therefore, in an embodiment, the serotype 12F glycoconjugate of the present invention comprises a serotype 12F capsular polysaccharide comprising between about 0.05 to about 25 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.05 to about 22.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.05 to about 15 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.05 to about 12.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.1 to about 25 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.1 to about 22.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.1 to about 15 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.1 to about 12.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.5 to about 25 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.5 to about 22.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.5 to about 15 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.5 to about 12.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 1 to about 25 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 1 to about 22.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 1 to about 15 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 1 to about 12.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 2 to about 25 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 2 to about 22.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 2 to about 15 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 2 to about 12.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 3 to about 25 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 3 to about 22.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 3 to about 15 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 3 to about 12.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 4 to about 25 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 4 to about 22.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 4 to about 15 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 4 to about 12.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 5 to about 25 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 5 to about 22.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 5 to about 15 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 5 to about 12.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 10 to about 25 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 10 to about 22.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 10 to about 15 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 10 to about 12.5 N-acetyl-D-fucosamine (D-FucNAc) residues in every 100 saccharide repeat units of the polysaccharide.

In an embodiment, the serotype 12F glycoconjugate of the present invention comprises a serotype 12F capsular polysaccharide comprising between about 0.05 to about 25 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.05 to about 22.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.05 to about 15 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.05 to about 12.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.1 to about 25 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.1 to about 22.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.1 to about 15 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.1 to about 12.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.5 to about 25 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.5 to about 22.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.5 to about 15 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.5 to about 12.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 1 to about 25 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 1 to about 22.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 1 to about 15 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 1 to about 12.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 2 to about 25 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 2 to about 22.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 2 to about 15 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 2 to about 12.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 3 to about 25 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 3 to about 22.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 3 to about 15 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 3 to about 12.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 4 to about 25 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 4 to about 22.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 4 to about 15 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 4 to about 12.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 5 to about 25 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 5 to about 22.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 5 to about 15 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 5 to about 12.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 10 to about 25 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 10 to about 22.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 10 to about 15 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 10 to about 12.5 N-acetyl-D-quinovosamine (D-QuiNAc) residues in every 100 saccharide repeat units of the polysaccharide.

In an embodiment, the serotype 12F glycoconjugate of the present invention comprises a serotype 12F capsular polysaccharide comprising between about 0.05 to about 25 N-acetyl-D-fucosamine (D-FucNAc) residues and between about 0.05 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.05 to about 22.5 N-acetyl-D-fucosamine (D-FucNAc) residues and between about 0.05 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.05 to about 15 N-acetyl-D-fucosamine (D-FucNAc) residues and between about 0.05 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.1 to about 12.5 N-acetyl-D-fucosamine (D-FucNAc) residues and between about 0.1 to about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.1 to about 25 N-acetyl-D-fucosamine (D-FucNAc) residues and between about 0.1 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.1 to about 22.5 N-acetyl-D-fucosamine (D-FucNAc) residues and between about 0.1 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.1 to about 15 N-acetyl-D-fucosamine (D-FucNAc) residues and between about 0.1 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.1 to about 12.5 N-acetyl-D-fucosamine (D-FucNAc) residues and between about 0.1 to about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.5 to about 25 N-acetyl-D-fucosamine (D-FucNAc) residues and between about 0.5 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.05 to about 22.5 N-acetyl-D-fucosamine (D-FucNAc) residues and between about 0.5 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.5 to about 15 N-acetyl-D-fucosamine (D-FucNAc) residues and between about 0.5 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.5 to about 12.5 N-acetyl-D-fucosamine (D-FucNAc) residues and between about 0.5 to about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 1 to about 25 N-acetyl-D-fucosamine (D-FucNAc) residues and between about 1 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 1 to about 22.5 N-acetyl-D-fucosamine (D-FucNAc) residues and between about 1 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 1 to about 15 N-acetyl-D-fucosamine (D-FucNAc) residues and between about 1 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 1 to about 12.5 N-acetyl-D-fucosamine (D-FucNAc) residues and between about 1 to about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 2 to about 25 N-acetyl-D-fucosamine (D-FucNAc) residues and between about 2 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 2 to about 22.5 N-acetyl-D-fucosamine (D-FucNAc) residues and between about 2 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 2 to about 15 N-acetyl-D-fucosamine (D-FucNAc) residues and between about 2 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 2 to about 12.5 N-acetyl-D-fucosamine (D-FucNAc) residues and between about 2 to about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 3 to about 25 N-acetyl-D-fucosamine (D-FucNAc) residues and between about 3 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 3 to about 22.5 N-acetyl-D-fucosamine (D-FucNAc) residues and between about 3 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 3 to about 15 N-acetyl-D-fucosamine (D-FucNAc) residues and between about 3 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 3 to about 12.5 N-acetyl-D-fucosamine (D-FucNAc) residues and between about 3 to about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 4 to about 25 N-acetyl-D-fucosamine (D-FucNAc) residues and between about 4 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 4 to about 22.5 N-acetyl-D-fucosamine (D-FucNAc) residues and between about 4 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 4 to about 15 N-acetyl-D-fucosamine (D-FucNAc) residues and between about 4 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 4 to about 12.5 N-acetyl-D-fucosamine (D-FucNAc) residues and between about 4 to about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 5 to about 25 N-acetyl-D-fucosamine (D-FucNAc) residues and between about 5 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 5 to about 22.5 N-acetyl-D-fucosamine (D-FucNAc) residues and between about 5 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 5 to about 15 N-acetyl-D-fucosamine (D-FucNAc) residues and between about 5 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 5 to about 12.5 N-acetyl-D-fucosamine (D-FucNAc) residues and between about 5 to about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 7.5 to about 25 N-acetyl-D-fucosamine (D-FucNAc) residues and between about 7.5 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 7.5 to about 22.5 N-acetyl-D-fucosamine (D-FucNAc) residues and between about 7.5 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 7.5 to about 15 N-acetyl-D-fucosamine (D-FucNAc) residues and between about 7.5 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 7.5 to about 12.5 N-acetyl-D-fucosamine (D-FucNAc) residues and between about 7.5 to about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 10 to about 25 N-acetyl-D-fucosamine (D-FucNAc) residues and between about 10 to about 25 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 10 to about 22.5 N-acetyl-D-fucosamine (D-FucNAc) residues and between about 10 to about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 10 to about 15 N-acetyl-D-fucosamine (D-FucNAc) residues and between about 10 to about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.05 to about 0.1 N-acetyl-D-fucosamine (D-FucNAc) residues and between about 0.05 to about 0.1 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.05 to about 0.5 N-acetyl-D-fucosamine (D-FucNAc) residues and between about 0.05 to about 0.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.05 to about 1 N-acetyl-D-fucosamine (D-FucNAc) residues and between about 0.05 to about 1 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.1 to about 0.2 N-acetyl-D-fucosamine (D-FucNAc) residues and between about 0.1 to about 0.2 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.1 to about 0.5 N-acetyl-D-fucosamine (D-FucNAc) residues and between about 0.1 to about 0.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising between about 0.1 to about 1 N-acetyl-D-fucosamine (D-FucNAc) residues and between about 0.1 to about 1 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 0.05 N-acetyl-D-fucosamine (D-FucNAc) residues and about 0.05 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 0.1 N-acetyl-D-fucosamine (D-FucNAc) residues and about 0.1 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 0.5 N-acetyl-D-fucosamine (D-FucNAc) residues and about 0.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 1 N-acetyl-D-fucosamine (D-FucNAc) residues and about 1 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 2 N-acetyl-D-fucosamine (D-FucNAc) residues and about 2 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 3 N-acetyl-D-fucosamine (D-FucNAc) residues and about 3 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 5 N-acetyl-D-fucosamine (D-FucNAc) residues and about 5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 7 N-acetyl-D-fucosamine (D-FucNAc) residues and about 7 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 12 N-acetyl-D-fucosamine (D-FucNAc) residues and about 12 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 12.5 N-acetyl-D-fucosamine (D-FucNAc) residues and about 12.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 13 N-acetyl-D-fucosamine (D-FucNAc) residues and about 13 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 14 N-acetyl-D-fucosamine (D-FucNAc) residues and about 14 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 15 N-acetyl-D-fucosamine (D-FucNAc) residues and about 15 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 22.5 N-acetyl-D-fucosamine (D-FucNAc) residues and about 22.5 N-acetyl-D-quinovosamine (D-QuiNAc) in every 100 saccharide repeat units of the polysaccharide.

In an embodiment, the serotype 12F glycoconjugate of the present invention comprises a serotype 12F capsular polysaccharide comprising about 25 N-acetyl-D-fucosamine (D-FucNAc) residues and about 25 N-acetyl-D-quinovosamine (D-QuiNAc) in every 100 saccharide repeat units of the polysaccharide.

The serotype 12F glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In a preferred embodiment, the serotype 12F glycoconjugate comprises less than about 50% of free serotype 12F polysaccharide compared to the total amount of serotype 12F polysaccharide. In a preferred embodiment, the serotype 12F glycoconjugate comprises less than about 25% of free serotype 12F polysaccharide compared to the total amount of serotype 12F polysaccharide. In an even preferred embodiment, the serotype 12F glycoconjugate comprises less than about 20% of free serotype 12F polysaccharide compared to the total amount of serotype 12F polysaccharide. In a yet preferred embodiment, the serotype 12F glycoconjugate comprises less than about 15% of free serotype 12F polysaccharide compared to the total amount of serotype 12F polysaccharide.

The serotype 12F glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In a preferred embodiment, at least 30% of the serotype 12F glycoconjugate has a K_{d} below or equal to 0.3 in a CL-4B column. In a preferred embodiment, at least 40% of the glycoconjugate has a K_{d} below or equal to 0.3 in a CL-4B column. In a preferred embodiment, at least 60% of the serotype 12F glycoconjugate has a K_{d} below or equal to 0.3 in a CL-4B column. In a preferred embodiment, between 50% and 80% of the serotype 12F glycoconjugate has a K_{d} below or equal to 0.3 in a CL-4B column. In a preferred embodiment, between 65% and 80% of the serotype 12F glycoconjugate has a K_{d} below or equal to 0.3 in a CL-4B column.

Carrier Proteins

Another component of the glycoconjugate of the disclosure is a carrier protein to which the saccharide is conjugated. The term “protein carrier” or “carrier protein” or “carrier” refers to any protein molecule that may be conjugated to an antigen (such as a capsular polysaccharide) against which an immune response is desired.

Conjugation to a carrier can enhance the immunogenicity of the antigen. Protein carriers for the antigens can be toxins, toxoids or any mutant cross-reactive material (CRM) of the toxin from tetanus, diphtheria, pertussis, Pseudomonas, E. coli, Staphylococcus and Streptococcus. In one embodiment, the carrier protein is CRM₁₉₇, derived from C. diphtheriae strain C7 (β197), which produces CRM₁₉₇ protein. This strain has ATCC accession No. 53281. A method for producing CRM₁₉₇ is described in U.S. Pat. No. 5,614,382. Alternatively, a fragment or epitope of the protein carrier or other immunogenic protein can be used. For example, a haptenic antigen can be coupled to a T-cell epitope of a bacterial toxin, toxoid or CRM. Other suitable carrier proteins include inactivated bacterial toxins such as cholera toxoid (e.g., as described in Int'l Patent Application No. WO 2004/083251), E. coli LT, E. coli ST, and exotoxin A from Pseudomonas aeruginosa. Bacterial outer membrane proteins such as outer membrane complex c (OMPC), porins, transferrin binding proteins, pneumolysin, pneumococcal surface protein A (PspA), pneumococcal adhesion protein (PsaA) or Haemophilus influenzae protein D can also be used. Other proteins, such as ovalbumin, keyhole limpet hemocyanin (KLH), bovine serum albumin (BSA) or purified protein derivative of tuberculin (PPD) also can be used as carrier proteins.

In a preferred embodiment, the carrier protein of the serotype 12F glycoconjugate of the invention is TT (tetanus toxoid), DT (Diphtheria toxoid), DT mutants (such as CRM₁₉₇), or a C5a peptidase from Streptococcus (SCP).

In a preferred embodiment, the carrier protein of the serotype 12F glycoconjugate of the invention is selected from the group consisting of TT (tetanus toxoid), DT (Diphtheria toxoid), DT mutants (such as CRM₁₉₇), and a C5a peptidase from Streptococcus (SCP).

In an embodiment, the carrier protein of the serotype 12F capsular polysaccharide glycoconjugate is DT (Diphtheria toxoid). In another embodiment, the carrier protein of the serotype 12F capsular polysaccharide glycoconjugate is TT (tetanus toxoid).

In another embodiment, the carrier protein of the serotype 12F capsular polysaccharide glycoconjugate is PD (H. influenzae protein D; see, e.g., EP0594610 B).

In a preferred embodiment, the carrier protein of the serotype 12F capsular polysaccharide glycoconjugate is CRM₁₉₇.

As discussed previously herein, the number of lysine residues in the carrier protein that become conjugated to the saccharide can be characterized as a range of conjugated lysines.

For example, in a given immunogenic composition, the CRM₁₉₇ may comprise 1 to 15 lysine residues out of 39 covalently linked to the saccharide. Another way to express this parameter is that about 2.5% to about 40% of CRM₁₉₇ lysines are covalently linked to the saccharide. For example, in a given immunogenic composition, the CRM₁₉₇ may comprise 1 to 20 lysine residues out of 39 covalently linked to the 12F saccharide. Another way to express this parameter is that about 2.5% to about 50% of CRM₁₉₇ lysines are covalently linked to the 12F saccharide.

### 3. Methods for Making Streptococcus pneumoniae Serotype 12F Glycoconjugates of the Invention

In an embodiment, the serotype 12F glycoconjugate of the present invention is prepared using reductive amination.

Reductive amination involves two steps, (1) oxidation (activation) of the purified saccharide, (2) reduction of the activated saccharide and a carrier protein (e.g., CRM₁₉₇, TT or SCP) to form a glycoconjugate (see e.g. WO2006/110381, WO2008/079653, WO2008/143709, WO2008/079732, WO2011/110531, WO2012/119972, WO2015110941, WO2015110940, WO2018/144439, WO2018/156491).

As mentioned above, before oxidation, sizing of the polysaccharide to a target molecular weight (MW) range can be performed.

Therefore, in an embodiment, the isolated 12F polysaccharide is sized before oxidation. In an embodiment, the isolated 12F polysaccharide is sized to any of the target molecular weight (MW) range defined above.

In one embodiment, the serotype 12F glycoconjugate of the present invention is prepared by a process comprising the step of: a) reacting a serotype 12F saccharide with a stable nitroxyl radical compound and an oxidant to produce an activated saccharide; and b) reacting the activated saccharide with a carrier protein.

In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO or 4-Amino-TEMPO, 4-Acetamido-2,2,6,6-tetramethylpiperidine 1-oxyl. Preferably said stable nitroxyl radical compound is TEMPO. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL. In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione.

Preferably said oxidant is N-Chlorosuccinimide.

In an aspect, said stable nitroxyl radical compound is 2,2,6,6-Tetramethyl-1-piperidinyloxy free radical (TEMPO) and said oxidant is N-Chlorosuccinimide (NCS).

In an aspect, step a) of the reaction is carried out in aqueous solvent. In another aspect, step a) is carried out in aprotic solvent. In an aspect, step a) is carried out in DMSO (dimethylsulfoxide), Dimethylacetamide (DMA), Sulfolane, N-Methyl-2-pyrrolidone (NMP), Hexamethylphosphoramide (HMPA) or in DMF (dimethylformamide) solvent. In an aspect, step a) is carried out in DMSO (dimethylsulfoxide).

In an aspect, the saccharide is reacted with 0.1 to 10 molar equivalents of oxidant. Preferably, the saccharide is reacted with 0.2 to 5, 0.5 to 2.5 or 0.5 to 1.5 molar equivalent of oxidant. In an aspect, the polysaccharide is reacted with about 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8 or 5 molar equivalent of oxidant.

In an aspect, the stable nitroxyl radical compound is present in a catalytic amount. In an aspect, the saccharide is reacted with less than about 0.3 molar equivalent of stable nitroxyl radical compound. In an aspect, the saccharide is reacted with less than about 0.005 molar equivalent of stable nitroxyl radical compound. In an aspect, the saccharide is reacted with about 0.005, 0.01, 0.05 or 0.1 molar equivalent of stable nitroxyl radical compound.

At the end of the reduction reaction, there may be unreacted aldehyde groups remaining in the conjugates, these may be capped using a suitable capping agent. In one embodiment this capping agent is sodium borohydride (NaBH₄).

In an embodiment capping is achieved by mixing the product of step c) with 0.5 to 20 molar equivalents of sodium borohydride. In an embodiment capping is achieved by mixing the product of step c) with 1 to 15 molar equivalents of sodium borohydride. In an embodiment capping is achieved by mixing the product of step c) with 0.5 to 5 molar equivalents of sodium borohydride. In an embodiment capping is achieved by mixing the product of step c) with 0.75 to 3 molar equivalents of sodium borohydride. In an embodiment capping is achieved by mixing the product of step c) with 1 molar equivalents of sodium borohydride. In an embodiment capping is achieved by mixing the product of step c) with 2 molar equivalents of sodium borohydride. In an embodiment capping is achieved by mixing the product of step c) with 3 molar equivalents of sodium borohydride.

In an embodiment, the serotype 12F glycoconjugate of the present invention is prepared by a process comprising the step of:
(a) reacting an isolated serotype 12F polysaccharide with an oxidizing agent;
(b) compounding the activated polysaccharide of step (a) with a carrier protein; and
(c) reacting the compounded activated polysaccharide and carrier protein with a reducing agent to form a glycoconjugate.

In an embodiment, the serotype 12F glycoconjugate of the present invention is prepared by a process comprising the step of:
(a) reacting an isolated serotype 12F polysaccharide with an oxidizing agent;
(a′) quenching the oxidation reaction by addition of a quenching agent;
(b) compounding the activated polysaccharide of step (a′) with a carrier protein; and
(c) reacting the compounded activated polysaccharide and carrier protein with a reducing agent to form a glycoconjugate.

Following the oxidation step (a) the saccharide is said to be activated and is referred to as “activated polysaccharide”.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate).

In an embodiment, the oxidizing agent is orthoperiodate.

In a preferred embodiment, the oxidizing agent is sodium periodate. In an embodiment, the periodate used for the oxidation is metaperiodate. In an embodiment the periodate used for the oxidation is sodium metaperiodate.

When a polysaccharide reacts with periodate, periodate oxidises vicinal hydroxyl groups to form carbonyl or aldehyde groups and causes cleavage of a C—C bond. For this reason, the term “reacting a polysaccharide with periodate” includes oxidation of vicinal hydroxyl groups by periodate.

In one embodiment step a) comprises reacting the polysaccharide with 0.01-2 molar equivalents of periodate. In one embodiment step a) comprises reacting the polysaccharide with 0.05-1.5 molar equivalents of periodate. In one embodiment step a) comprises reacting the polysaccharide with 0.1-1.0 molar equivalents of periodate. In one embodiment step a) comprises reacting the polysaccharide with 0.01-0.5 molar equivalents of periodate. In one embodiment step a) comprises reacting the polysaccharide with 0.1-0.5 molar equivalents of periodate.

In one embodiment, the quenching agent of step a′) is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

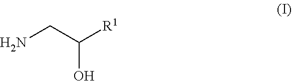

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

Preferably, the quenching agent is a compound of formula (II):

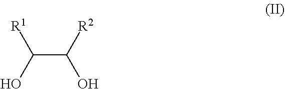

wherein R¹ and R² are each independently selected from H, methyl, ethyl, propyl or isopropyl.

In a preferred embodiment, the quenching agent is glycerol, ethylene glycol, propan-1,2-diol, butan-1,2-diol or butan-2,3-diol, or ascorbic acid. In an even preferred embodiment, the quenching agent is butan-2,3-diol.

In a preferred embodiment the degree of oxidation of the activated serotype 12F polysaccharide is between 2 and 30.

In an embodiment, the reduction reaction (c) is carried out in aqueous solvent. In an embodiment, the reduction reaction (c) is carried out in aprotic solvent.

In an embodiment, the reduction reaction (c) is carried out in the presence of dimethylsulfoxide (DMSO) or dimethylformamide (DMF). In an embodiment, the reduction reaction (c) is carried out in the presence of dimethylformamide (DMF). In an embodiment, the reduction reaction (c) is carried out in the presence of dimethylsulphoxide (DMSO).

In one embodiment the reduction reaction (c) is carried out in a solution consisting essentially of dimethylsulphoxide (DMSO) or dimethylformamide (DMF). In one embodiment the reduction reaction (c) is carried out in a solution consisting essentially of dimethylformamide (DMF). In one embodiment the reduction reaction (c) is carried out in a solution consisting essentially of dimethylsulphoxide (DMSO).

In an embodiment, the reduction reaction (c) is carried out in DMSO (dimethylsulfoxide) or in DMF (dimethylformamide)) solvent. In an embodiment, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium triacetoxyborohydride. In a preferred embodiment, the reducing agent is sodium cyanoborohydride. In an embodiment, the reducing agent is sodium cyanoborohydride in the present of nickel (see WO2018144439).

In one embodiment between 1.0 and 20 molar equivalents of reducing agent is used in step c). In one embodiment between 2 and 15 molar equivalents of reducing agent is used in step c). In one embodiment between 5 and 15 molar equivalents of reducing agent is used in step c).

At the end of the reduction reaction, there may be unreacted aldehyde groups remaining in the conjugates, these may be capped using a suitable capping agent. In one embodiment this capping agent is sodium borohydride (NaBH₄).

In an embodiment capping is achieved by mixing the product of step c) with 1 to 20 molar equivalents of sodium borohydride. In an embodiment capping is achieved by mixing the product of step c) with 2 to 15 molar equivalents of sodium borohydride. In an embodiment capping is achieved by mixing the product of step c) with 5 to 10 molar equivalents of sodium borohydride.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

### 4. Immunogenic Compositions

In an embodiment the invention relates to an immunogenic composition comprising S. pneumoniae serotype 12F saccharide of the invention.

In an embodiment the invention relates to an immunogenic composition comprising a Streptococcus pneumoniae serotype 12F glycoconjugate of the invention.

In an embodiment the invention relates to an immunogenic composition comprising a Streptococcus pneumoniae serotype 12F glycoconjugate of the invention and comprising from 1 to 25 different glycoconjugates.

In an embodiment the invention relates to an immunogenic composition comprising a Streptococcus pneumoniae serotype 12F glycoconjugate of the invention and comprising from 1 to 25 glycoconjugates from different serotypes of S. pneumoniae (1 to 25 pneumococcal conjugates). In one embodiment the invention relates to an immunogenic composition comprising glycoconjugates from 7, 8, 9, 10, 11, 12, 13, 14, 15, 16, 17, 18, 19, 20, 21, 22, 23, 24 or 25 different serotypes of S. pneumoniae. In one embodiment the immunogenic composition comprises glycoconjugates from 16 or 20 different serotypes of S. pneumoniae. In an embodiment the immunogenic composition is a 7, 8, 9, 10, 11, 12, 13, 14, 15, 16, 17, 18, 19 or 20-valent pneumococcal conjugate compositions. In an embodiment the immunogenic composition is a 14, 15, 16, 17, 18 or 19-valent pneumococcal conjugate compositions. In an embodiment the immunogenic composition is a 16-valent pneumococcal conjugate composition. In an embodiment the immunogenic composition is a 19-valent pneumococcal conjugate composition. In an embodiment the immunogenic composition is a 20-valent pneumococcal conjugate composition.

In an embodiment the invention relates to an immunogenic composition comprising a Streptococcus pneumoniae serotype 12F glycoconjugate of the invention and comprising from 26 to 45 glycoconjugates from different serotypes of S. pneumoniae (26 to 45 pneumococcal conjugates). In one embodiment the invention relates to an immunogenic composition comprising glycoconjugates from 26, 27, 28, 29, 30, 31, 32, 33, 34, 35, 36, 37, 38, 39, 40, 41, 42, 43, 44 or 45 different serotypes of S. pneumoniae. In one embodiment the immunogenic composition comprises glycoconjugates from 35 or 45 different serotypes of S. pneumoniae. In an embodiment the immunogenic composition is a 35, 36, 37, 38, 39, 40, 41, 42, 43, 44 or 45-valent pneumococcal conjugate compositions. In an embodiment the immunogenic composition is a 40, 41, 42, 43, 44 or 45-valent pneumococcal conjugate compositions. In an embodiment the immunogenic composition is a 40-valent pneumococcal conjugate composition. In an embodiment the immunogenic composition is a 41-valent pneumococcal conjugate composition. In an embodiment the immunogenic composition is a 42-valent pneumococcal conjugate composition. In an embodiment the immunogenic composition is a 43-valent pneumococcal conjugate composition. In an embodiment the immunogenic composition is a 44-valent pneumococcal conjugate composition. In an embodiment the immunogenic composition is a 45-valent pneumococcal conjugate composition.

In an embodiment the invention relates to an immunogenic composition comprising a Streptococcus pneumoniae serotype 12F glycoconjugate of the invention and further comprising glycoconjugates from S. pneumoniae serotypes 4, 6B, 9V, 14, 18C, 19F and 23F.

In an embodiment said immunogenic composition comprises in addition glycoconjugates from S. pneumoniae serotypes 1, 5 and 7F.

In an embodiment any of the immunogenic compositions above comprises in addition glycoconjugates from S. pneumoniae serotype 3.

In an embodiment any of the immunogenic compositions above comprises in addition glycoconjugates from S. pneumoniae serotypes 6A and 19A.

In an embodiment any of the immunogenic compositions above comprise in addition a glycoconjugates from S. pneumoniae serotype 22F and 33F.

In an embodiment any of the immunogenic compositions above comprise in addition a glycoconjugates from S. pneumoniae serotypes 8, 10A, 11A and 15B.

In an embodiment any of the immunogenic compositions above comprise in addition a glycoconjugates from S. pneumoniae serotype 2.

In an embodiment any of the immunogenic compositions above comprise in addition a glycoconjugates from S. pneumoniae serotype 9N.

In an embodiment any of the immunogenic compositions above comprise in addition a glycoconjugates from S. pneumoniae serotype 17F.

In an embodiment any of the immunogenic compositions above comprise in addition a glycoconjugates from S. pneumoniae serotype 20.

In an embodiment any of the immunogenic compositions above comprise in addition a glycoconjugates from S. pneumoniae serotype 15C.

In an embodiment the invention relates to an immunogenic composition comprising a Streptococcus pneumoniae serotype 12F glycoconjugate of the invention and further comprising glycoconjugates from S. pneumoniae serotypes 4, 6B, 9V, 14, 18C, 19F and 23F. In an embodiment the immunogenic composition is an 8-valent pneumococcal conjugate compositions.

In an embodiment the invention relates to an immunogenic composition comprising a Streptococcus pneumoniae serotype 12F glycoconjugate of the invention and further comprising glycoconjugates from S. pneumoniae serotypes 1, 4, 5, 6B, 7F, 9V, 14, 18C, 19F and 23F. In an embodiment the immunogenic composition is an 11-valent pneumococcal conjugate compositions.

In an embodiment the invention relates to an immunogenic composition comprising a Streptococcus pneumoniae serotype 12F glycoconjugate of the invention and further comprising glycoconjugates from S. pneumoniae serotypes 1, 3, 4, 5, 6A, 6B, 7F, 9V, 14, 18C, 19A, 19F, 22F, 23F and 33F. In an embodiment the immunogenic composition is a 16-valent pneumococcal conjugate compositions.

In an embodiment the invention relates to an immunogenic composition comprising a Streptococcus pneumoniae serotype 12F glycoconjugate of the invention and further comprising glycoconjugates from S. pneumoniae serotypes 1, 3, 4, 5, 6A, 6B, 7F, 8, 9V, 10A, 11A, 14, 15B, 18C, 19A, 19F, 22F, 23F and 33F. In an embodiment the immunogenic composition is a 20-valent pneumococcal conjugate compositions.

In an embodiment the invention relates to an immunogenic composition comprising a Streptococcus pneumoniae serotype 12F glycoconjugate of the invention and further comprising glycoconjugates from S. pneumoniae serotypes 1, 3, 4, 5, 6A, 6B, 7F, 8, 9V, 10A, 11A, 14, 15C, 18C, 19A, 19F, 22F, 23F and 33F. In an embodiment the immunogenic composition is a 20-valent pneumococcal conjugate compositions.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugate from S. pneumoniae serotype 12F is conjugated to CRM₁₉₇.

In an embodiment of any of the above immunogenic compositions, the glycoconjugate from S. pneumoniae serotype 22F is conjugated to CRM₁₉₇. In an embodiment of any of the above immunogenic compositions, the glycoconjugate from S. pneumoniae serotype 33F is conjugated to CRM₁₉₇. In an embodiment of any of the above immunogenic compositions, the glycoconjugate from S. pneumoniae serotype 15B is conjugated to CRM₁₉₇. In an embodiment of any of the above immunogenic compositions, the glycoconjugate from S. pneumoniae serotype 10A is conjugated to CRM₁₉₇. In an embodiment of any of the above immunogenic compositions, the glycoconjugate from S. pneumoniae serotype 11A is conjugated to CRM₁₉₇. In an embodiment of any of the above immunogenic compositions, the glycoconjugate from S. pneumoniae serotype 8 is conjugated to CRM₁₉₇. In an embodiment of any of the above immunogenic compositions, the glycoconjugates from S. pneumoniae serotypes 4, 6B, 9V, 14, 18C, 19F and 23F are conjugated to CRM₁₉₇. In an embodiment of any of the above immunogenic compositions, the glycoconjugates from S. pneumoniae serotypes 1, 5 and 7F are conjugated to CRM₁₉₇. In an embodiment of any of the above immunogenic compositions, the glycoconjugates from S. pneumoniae serotypes 6A and 19A are conjugated to CRM₁₉₇. In an embodiment of any of the above immunogenic compositions, the glycoconjugate from S. pneumoniae serotype 3 is conjugated to CRM₁₉₇. In an embodiment of any of the above immunogenic compositions, the glycoconjugate from S. pneumoniae serotype 15C is conjugated to CRM₁₉₇.

In an embodiment of any of the above immunogenic compositions, the glycoconjugates of any of the above immunogenic compositions are all individually conjugated to CRM₁₉₇.

In an embodiment the above immunogenic compositions comprise from 8 to 20 different serotypes of S. pneumoniae. In an embodiment the above immunogenic compositions comprise from 21 to 45 different serotypes of S. pneumoniae.

Compositions of the invention may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

The immunogenic compositions of the present disclosure can be used to protect or treat a human susceptible to bacterial infection, e.g., by a S. pneumoniae bacteria, by means of administering the immunogenic compositions via a systemic, dermal or mucosal route, or can be used to generate a polyclonal or monoclonal antibody preparation that could be used to confer passive immunity on another subject. These administrations can include injection via the intramuscular, intraperitoneal, intradermal or subcutaneous routes; or via mucosal administration to the oral/alimentary, respiratory or genitourinary tracts. Immunogenic compositions may also be used to generate antibodies that are functional as measured by the killing of bacteria in either an animal efficacy model or via an opsonophagocytic killing assay.

Optimal amounts of components for a particular immunogenic composition can be ascertained by standard studies involving observation of appropriate immune responses in subjects. Following an initial vaccination, subjects can receive one or several booster immunizations adequately spaced.

In some embodiments, the immunogenic compositions disclosed herein may further comprise at least one adjuvant. In some embodiments, the immunogenic compositions disclosed herein may further comprise one adjuvant. In some embodiments, the immunogenic compositions disclosed herein may further comprise two adjuvants. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant. In a preferred embodiment, the immunogenic compositions disclosed herein comprise aluminum phosphate as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

### 5. Uses of the Immunogenic Compositions of the Invention

The S. pneumoniae serotype 12F saccharide or S. pneumoniae serotype 12F glycoconjugate disclosed herein may be use as antigens. For example, they may be part of a vaccine.

Therefore, in an embodiment, the immunogenic compositions of the invention are for use as a medicament.

In an embodiment, the immunogenic compositions of the invention are for use as a vaccine. Therefore, in an embodiment, the immunogenic compositions described herein are for use in generating an immune response in a subject. In one aspect, the subject is a mammal, such as a human, cat, sheep, pig, horse, bovine or dog. In one aspect, the subject is a human.

The immunogenic compositions described herein may be used in therapeutic or prophylactic methods for preventing, treating or ameliorating a bacterial infection, disease or condition in a subject. In particular, immunogenic compositions described herein may be used to prevent, treat or ameliorate a S. pneumoniae serotype 12F infection, disease or condition in a subject.

Thus, in one aspect, the disclosure provides a method of preventing, treating or ameliorating an infection, disease or condition associated with S. pneumoniae serotype 12F in a subject, comprising administering to the subject an immunologically effective amount of an immunogenic composition of the disclosure.

In some such embodiments, the infection, disease or condition is pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection or brain abscess.

In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess.

In an embodiment, the disclosure provides a method of inducing an immune response to S. pneumoniae serotype 12F in a subject comprising administering to the subject an immunologically effective amount of an immunogenic composition of the invention. In one aspect, the subject is a mammal, such as a human, cat, sheep, pig, horse, bovine or dog. In one aspect, the subject is a human.

In an embodiment, the immunogenic compositions disclosed herein are for use as a vaccine. In such embodiments the immunogenic compositions described herein may be used to prevent S. pneumoniae serotype 12F infection in a subject. Thus, in one aspect, the invention provides a method of preventing an infection by S. pneumoniae serotype 12F in a subject comprising administering to the subject an immunologically effective amount of an immunogenic composition of the disclosure. In some such embodiments, the infection is pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection or brain abscess. In one aspect, the subject is a mammal, such as a human, cat, sheep, pig, horse, bovine or dog. In one aspect, the subject is a human. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject is a mammal, such as a human, cat, sheep, pig, horse, bovine or dog. In one aspect, the subject is a human.

The immunogenic composition of the present disclosure can be used to protect or treat a human susceptible to a S. pneumoniae serotype 12F infection, by means of administering the immunogenic composition via a systemic or mucosal route. In an embodiment, the immunogenic composition of the invention is administered by intramuscular, intraperitoneal, intradermal or subcutaneous routes. In an embodiment, the immunogenic composition of the invention is administered by intramuscular, intraperitoneal, intradermal or subcutaneous injection. In an embodiment, the immunogenic composition of the invention is administered by intramuscular or subcutaneous injection. In an embodiment, the immunogenic composition of the invention is administered by intramuscular injection. In an embodiment, the immunogenic composition of the invention is administered by subcutaneous injection.

### 6. Analytical Methods

In an embodiment the invention relates to a method of detecting the presence of 4-keto-N-acetyl-quinovosamine residues in an isolated S. pneumoniae serotype 12F polysaccharide, said method comprising the step of: a) isolating an S. pneumoniae serotype 12F polysaccharide and b) detecting the presence of 4-keto-N-acetyl-quinovosamine residues in said polysaccharide.

In an embodiment the presence of 4-keto-N-acetyl-quinovosamine residues is detected by NMR or Mass Spectrometry (MS). In an embodiment the presence of 4-keto-N-acetyl-quinovosamine residues is detected by NMR. In an embodiment, the presence of 4-keto-N-acetyl-quinovosamine residues is detected by 1 D NMR. In an embodiment, the presence of 4-keto-N-acetyl-quinovosamine residues is detected by 1D ¹H or 1D ¹³C NMR. In an embodiment, the presence of 4-keto-N-acetyl-quinovosamine residues is detected by 2D NMR. In an embodiment, the presence of 4-keto-N-acetyl-quinovosamine residues is detected by Heteronuclear Single Quantum Coherence Spectroscopy (HSQC), Heteronuclear multiple-bond correlation spectroscopy (HMBC), Nuclear Overhauser Effect Spectroscopy (NOESY), Correlation spectroscopy (COSY), Total Correlation Spectroscopy (TOCSY) or Heteronuclear Single Quantum Coherence Spectroscopy-Total Correlation Spectroscopy (HSQC-TOCSY).

In an embodiment, the presence of 4-keto-N-acetyl-quinovosamine residues is detected by 1D ¹H, 2D ¹H-¹³C Heteronuclear Single Quantum Coherence Spectroscopy (HSQC), 2D ¹H-¹³C Heteronuclear multiple-bond correlation spectroscopy (HMBC), 2D ¹H-¹³C Nuclear Overhauser Effect Spectroscopy (NOESY), 2D ¹H-¹³C Correlation spectroscopy (COSY), 2D ¹H-¹³C Total Correlation Spectroscopy (TOCSY), 2D ¹H-¹³C Heteronuclear Single Quantum Coherence Spectroscopy-Total Correlation Spectroscopy (HSQC-TOCSY) or 1D ¹³C NMR. In a preferred embodiment, the presence of 4-keto-N-acetyl-quinovosamine residues is detected by 1D ¹H, 2D ¹H-¹³C Heteronuclear Single Quantum Coherence Spectroscopy (HSQC), or 1D ¹³C NMR.

In an embodiment, the presence of 4-keto-N-acetyl-quinovosamine residues is detected by 2D ¹H-¹³C Heteronuclear Single Quantum Coherence Spectroscopy (HSQC).

In an embodiment the presence of 4-keto-N-acetyl-quinovosamine residues is detected by Mass Spectrometry (MS). In an embodiment the presence of 4-keto-N-acetyl-quinovosamine residues is detected by Tandem Mass Spectrometry (MS/MS). In an embodiment the presence of 4-keto-N-acetyl-quinovosamine residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the presence of 4-keto-N-acetyl-quinovosamine residues is detected by Size-Exclusion Chromatography combined with Mass Spectrometry (SEC/MS).

In an embodiment the presence of 4-keto-N-acetyl-quinovosamine residues is detected by Gas Chromatography-Mass Spectrometry (GC-MS). In an embodiment the presence of 4-keto-N-acetyl-quinovosamine residues is detected by Liquid Chromatography-Mass Spectrometry (LC-MS). In an embodiment the presence of 4-keto-N-acetyl-quinovosamine residues is detected by Capillary Electrophoresis-Mass Spectrometry (CE-MS). In an embodiment the presence of 4-keto-N-acetyl-quinovosamine residues is detected by Ion Mobility Spectrometry-Mass Spectrometry (IMS/MS). In an embodiment the presence of 4-keto-N-acetyl-quinovosamine residues is detected by Hydrophilic Interaction Liquid Chromatography-Mass Spectrometry (HILIC-LC/MS).

In an embodiment, the invention relates to a method of determining the amount of 4-keto-N-acetyl-quinovosamine residues in an isolated S. pneumoniae serotype 12F polysaccharide, said method comprising the step of: a) isolating an S. pneumoniae serotype 12F polysaccharide and b) measuring the amount of 4-keto-N-acetyl-quinovosamine residues in said polysaccharide.

In an embodiment the amount of 4-keto-N-acetyl-quinovosamine residues is determined by NMR. In an embodiment the amount of 4-keto-N-acetyl-quinovosamine residues is determined by 1 D NMR. In an embodiment, the amount of 4-keto-N-acetyl-quinovosamine residues is determined by 1D ¹H or 1D ¹³C NMR. In a preferred embodiment the amount of 4-keto-N-acetyl-quinovosamine residues is determined by 1D ¹H NMR. In an embodiment, the amount of 4-keto-N-acetyl-quinovosamine residues is determined by integration or deconvolution of 1D ¹H spectra.

In an embodiment the amount of 4-keto-N-acetyl-quinovosamine residues is determined by 2D NMR. In an embodiment the amount of 4-keto-N-acetyl-quinovosamine residues is determined by crosspeak integration of 2D ¹H-¹³C HSQC spectra.

In an embodiment the amount of 4-keto-N-acetyl-quinovosamine residues is determined by Mass Spectrometry (MS). In an embodiment the amount of 4-keto-N-acetyl-quinovosamine residues is determined by Tandem Mass Spectrometry (MS/MS). In an embodiment the amount of 4-keto-N-acetyl-quinovosamine residues is determined by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the amount of 4-keto-N-acetyl-quinovosamine residues is determined by Size-Exclusion Chromatography combined with Mass Spectrometry (SEC/MS).

In an embodiment the amount of 4-keto-N-acetyl-quinovosamine residues is determined by Gas Chromatography-Mass Spectrometry (GC-MS). In an embodiment the amount of 4-keto-N-acetyl-quinovosamine residues is determined by Liquid Chromatography-Mass Spectrometry (LC-MS). In an embodiment the amount of 4-keto-N-acetyl-quinovosamine residues is determined by Capillary Electrophoresis-Mass Spectrometry (CE-MS). In an embodiment the amount of 4-keto-N-acetyl-quinovosamine residues is determined by Ion Mobility Spectrometry-Mass Spectrometry (IMS/MS). In an embodiment the amount of 4-keto-N-acetyl-quinovosamine residues is determined by Hydrophilic Interaction Liquid Chromatography-Mass Spectrometry (HILIC-LC/MS).

In an embodiment, the invention relates to a method of detecting the presence of N-acetyl-D-fucosamine (D-FucNAc) residues in a reduced serotype 12F polysaccharide, said method comprising the step of: a) reacting an isolated S. pneumoniae serotype 12F polysaccharide with a reducing agent and b) detecting the presence of N-acetyl-D-fucosamine (D-FucNAc) residues in said reduced polysaccharide.

In an embodiment the presence of N-acetyl-D-fucosamine (D-FucNAc) residues is detected by NMR. In an embodiment the presence of N-acetyl-D-fucosamine (D-FucNAc) residues is detected by 2D NMR.

In a preferred embodiment, the presence of N-acetyl-D-fucosamine (D-FucNAc) residues is detected by 2D ¹H-¹³C HSQC NMR.

In an embodiment, the presence of N-acetyl-D-fucosamine (D-FucNAc) residues is detected by Mass Spectrometry (MS). In an embodiment, the presence of N-acetyl-D-fucosamine (D-FucNAc) residues is detected by Tandem Mass Spectrometry (MS/MS). In an embodiment, the presence of N-acetyl-D-fucosamine (D-FucNAc) residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment, the presence of N-acetyl-D-fucosamine (D-FucNAc) residues is detected by Size-Exclusion Chromatography combined with Mass Spectrometry (SEC/MS).

In an embodiment, the presence of N-acetyl-D-fucosamine (D-FucNAc) residues is detected by Gas Chromatography-Mass Spectrometry (GC-MS). In an embodiment, the presence of N-acetyl-D-fucosamine (D-FucNAc) residues is detected by Liquid Chromatography-Mass Spectrometry (LC-MS). In an embodiment, the presence of N-acetyl-D-fucosamine (D-FucNAc) residues is detected by Capillary Electrophoresis-Mass Spectrometry (CE-MS). In an embodiment, the presence of N-acetyl-D-fucosamine (D-FucNAc) residues is detected by Ion Mobility Spectrometry-Mass Spectrometry (IMS/MS). In an embodiment, the presence of N-acetyl-D-fucosamine (D-FucNAc) residues is detected by Hydrophilic Interaction Liquid Chromatography-Mass Spectrometry (HILIC-LC/MS).

In an embodiment, said reducing agent is sodium borohydride (NaBH₄).

In an embodiment, said isolated S. pneumoniae serotype 12F polysaccharide has been previously treated with an oxidizing agent. In an embodiment, the oxidizing agent is any oxidizing agent which oxidizes a terminal hydroxyl group to an aldehyde. In an embodiment, the oxidizing agent is periodate. In an embodiment, the oxidizing agent is orthoperiodate. In a preferred embodiment, the oxidizing agent is sodium periodate. In an embodiment, the oxidizing agent is metaperiodate. In a preferred embodiment the oxidizing agent is sodium metaperiodate.

In an embodiment, said isolated S. pneumoniae serotype 12F polysaccharide has been previously treated with a stable nitroxyl radical compound and an oxidant. In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO or 4-Acetamido-2,2,6,6-tetramethylpiperidine 1-oxyl. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL. In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In an aspect, said stable nitroxyl radical compound is 2,2,6,6-Tetramethyl-1-piperidinyloxy free radical (TEMPO) and said oxidant is N-Chlorosuccinimide (NCS).

In an embodiment, the invention relates to a method of detecting the presence of N-acetyl-D-quinovosamine (D-QuiNAc) residues in a reduced serotype 12F polysaccharide, said method comprising the step of: a) reacting an isolated S. pneumoniae serotype 12F polysaccharide with a reducing agent and b) detecting the presence of N-acetyl-D-quinovosamine (D-QuiNAc) residues in said reduced polysaccharide.

In an embodiment the presence of N-acetyl-D-quinovosamine (D-QuiNAc) residues is detected by NMR. In an embodiment the presence of N-acetyl-D-quinovosamine (D-QuiNAc) residues is detected by 2D NMR.

In an embodiment the presence of N-acetyl-D-quinovosamine (D-QuiNAc) residues is detected by Heteronuclear Single Quantum Coherence Spectroscopy (HSQC), Heteronuclear multiple-bond correlation spectroscopy (HMBC), Correlation spectroscopy (COSY), and/or Heteronuclear Single Quantum Coherence Spectroscopy-Total Correlation Spectroscopy (HSQC-TOCSY).

In a preferred embodiment, the presence of N-acetyl-D-quinovosamine (D-QuiNAc) residues is detected by 2D ¹H-¹³C HSQC NMR.

In an embodiment the presence of N-acetyl-D-quinovosamine (D-QuiNAc) residues is detected by Mass Spectrometry (MS). In an embodiment the presence of N-acetyl-D-quinovosamine (D-QuiNAc) residues is detected by Tandem Mass Spectrometry (MS/MS).

In an embodiment the presence of N-acetyl-D-quinovosamine (D-QuiNAc) residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the presence of N-acetyl-D-quinovosamine (D-QuiNAc) residues is detected by Size-Exclusion Chromatography combined with Mass Spectrometry (SEC/MS).

In an embodiment the presence of N-acetyl-D-quinovosamine (D-QuiNAc) residues is detected by Gas Chromatography-Mass Spectrometry (GC-MS). In an embodiment the presence of N-acetyl-D-quinovosamine (D-QuiNAc) residues is detected by Liquid Chromatography-Mass Spectrometry (LC-MS). In an embodiment the presence of N-acetyl-D-quinovosamine (D-QuiNAc) residues is detected by Capillary Electrophoresis-Mass Spectrometry (CE-MS). In an embodiment the presence of N-acetyl-D-quinovosamine (D-QuiNAc) residues is detected by Ion Mobility Spectrometry-Mass Spectrometry (IMS/MS). In an embodiment the presence of N-acetyl-D-quinovosamine (D-QuiNAc) residues is detected by Hydrophilic Interaction Liquid Chromatography-Mass Spectrometry (HILIC-LC/MS).

In an embodiment, said reducing agent is sodium borohydride (NaBH₄).

In an embodiment, said isolated S. pneumoniae serotype 12F polysaccharide has been previously treated with an oxidizing agent. In an embodiment, the oxidizing agent is any oxidizing agent which oxidizes a terminal hydroxyl group to an aldehyde. In an embodiment, the oxidizing agent is periodate. In an embodiment, the oxidizing agent is orthoperiodate. In a preferred embodiment, the oxidizing agent is sodium periodate. In an embodiment, the oxidizing agent is metaperiodate. In a preferred embodiment the oxidizing agent is sodium metaperiodate.

In an embodiment, said isolated S. pneumoniae serotype 12F polysaccharide has been previously treated with a stable nitroxyl radical compound and an oxidant. In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO or 4-Acetamido-2,2,6,6-tetramethylpiperidine 1-oxyl. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL. In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In an aspect, said stable nitroxyl radical compound is 2,2,6,6-Tetramethyl-1-piperidinyloxy free radical (TEMPO) and said oxidant is N-Chlorosuccinimide (NCS).

In an embodiment, the invention relates to a method of detecting the presence of N-acetyl-D-fucosamine (D-FucNAc) and N-acetyl-D-quinovosamine (D-QuiNAc) residues in a reduced serotype 12F polysaccharide, said method comprising the step of: a) reacting an isolated S. pneumoniae serotype 12F polysaccharide with a reducing agent and b) detecting the presence of N-acetyl-D-fucosamine (D-FucNAc) and N-acetyl-D-quinovosamine (D-QuiNAc) residues in said reduced polysaccharide.

In an embodiment the presence of N-acetyl-D-fucosamine (D-FucNAc) and N-acetyl-D-quinovosamine (D-QuiNAc) residues is detected by NMR.

In a preferred embodiment, the presence of N-acetyl-D-fucosamine (D-FucNAc) and N-acetyl-D-quinovosamine (D-QuiNAc) residues is detected by 2D-NMR.

In an embodiment the presence of N-acetyl-D-fucosamine (D-FucNAc) and N-acetyl-D-quinovosamine (D-QuiNAc) residues is detected by Mass Spectrometry (MS). In an embodiment the presence of N-acetyl-D-fucosamine (D-FucNAc) and N-acetyl-D-quinovosamine (D-QuiNAc) residues is detected by Tandem Mass Spectrometry (MS/MS).

In an embodiment the presence of N-acetyl-D-fucosamine (D-FucNAc) and N-acetyl-D-quinovosamine (D-QuiNAc) residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the presence of N-acetyl-D-fucosamine (D-FucNAc) and N-acetyl-D-quinovosamine (D-QuiNAc) residues is detected by Size-Exclusion Chromatography combined with Mass Spectrometry (SEC/MS).

In an embodiment the presence of N-acetyl-D-fucosamine (D-FucNAc) and N-acetyl-D-quinovosamine (D-QuiNAc) residues is detected by Gas Chromatography-Mass Spectrometry (GC-MS). In an embodiment the presence of N-acetyl-D-fucosamine (D-FucNAc) and N-acetyl-D-quinovosamine (D-QuiNAc) residues is detected by Liquid Chromatography-Mass Spectrometry (LC-MS). In an embodiment the presence of N-acetyl-D-fucosamine (D-FucNAc) and N-acetyl-D-quinovosamine (D-QuiNAc) residues is detected by Capillary Electrophoresis-Mass Spectrometry (CE-MS). In an embodiment the presence of N-acetyl-D-fucosamine (D-FucNAc) and N-acetyl-D-quinovosamine (D-QuiNAc) residues is detected by Ion Mobility Spectrometry-Mass Spectrometry (IMS/MS). In an embodiment the presence of N-acetyl-D-fucosamine (D-FucNAc) and N-acetyl-D-quinovosamine (D-QuiNAc) residues is detected by Hydrophilic Interaction Liquid Chromatography-Mass Spectrometry (HILIC-LC/MS).

In an embodiment, said reducing agent is sodium borohydride (NaBH₄).

In an embodiment, said isolated S. pneumoniae serotype 12F polysaccharide has been previously treated with an oxidizing agent. In an embodiment, the oxidizing agent is any oxidizing agent which oxidizes a terminal hydroxyl group to an aldehyde. In an embodiment, the oxidizing agent is periodate. In an embodiment, the oxidizing agent is orthoperiodate. In a preferred embodiment, the oxidizing agent is sodium periodate. In an embodiment, the oxidizing agent is metaperiodate. In a preferred embodiment the oxidizing agent is sodium metaperiodate.

In an embodiment, said isolated S. pneumoniae serotype 12F polysaccharide has been previously treated with a stable nitroxyl radical compound and an oxidant. In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO or 4-Acetamido-2,2,6,6-tetramethylpiperidine 1-oxyl. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL. In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In an aspect, said stable nitroxyl radical compound is 2,2,6,6-Tetramethyl-1-piperidinyloxy free radical (TEMPO) and said oxidant is N-Chlorosuccinimide (NCS).

In an embodiment the invention relates to a method of detecting the presence of N-acetyl-D-fucosamine (D-FucNAc) and/or N-acetyl-D-quinovosamine (D-QuiNAc) residues in S. pneumoniae serotype 12F glycoconjugate, said method comprising the step of: a) preparing a S. pneumoniae serotype 12F glycoconjugate and b) detecting the presence of N-acetyl-D-fucosamine (D-FucNAc) and/or N-acetyl-D-quinovosamine (D-QuiNAc) residues in said glycoconjugate.

In an embodiment the presence of N-acetyl-D-fucosamine (D-FucNAc) and/or N-acetyl-D-quinovosamine (D-QuiNAc) residues is detected by NMR. In an embodiment the presence of N-acetyl-D-fucosamine (D-FucNAc) and/or N-acetyl-D-quinovosamine (D-QuiNAc) residues is detected by 2D NMR.

In an embodiment the presence of N-acetyl-D-fucosamine (D-FucNAc) and/or N-acetyl-D-quinovosamine (D-QuiNAc) residues is detected by Heteronuclear Single Quantum Coherence Spectroscopy (HSQC), Heteronuclear multiple-bond correlation spectroscopy (HMBC), Correlation spectroscopy (COSY), and/or Heteronuclear Single Quantum Coherence Spectroscopy-Total Correlation Spectroscopy (HSQC-TOCSY).

In a preferred embodiment, the presence of N-acetyl-D-fucosamine (D-FucNAc) and/or N-acetyl-D-quinovosamine (D-QuiNAc) residues is detected by 2D ¹H-¹³C HSQC NMR.

In an embodiment the presence of N-acetyl-D-fucosamine (D-FucNAc) and/or N-acetyl-D-quinovosamine (D-QuiNAc) residues is detected by Mass Spectrometry (MS). In an embodiment the presence of N-acetyl-D-fucosamine (D-FucNAc) and/or N-acetyl-D-quinovosamine (D-QuiNAc) residues is detected by Tandem Mass Spectrometry (MS/MS).

In an embodiment the presence of N-acetyl-D-fucosamine (D-FucNAc) and/or N-acetyl-D-quinovosamine (D-QuiNAc) residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the presence of N-acetyl-D-fucosamine (D-FucNAc) and/or N-acetyl-D-quinovosamine (D-QuiNAc) residues is detected by Size-Exclusion Chromatography combined with Mass Spectrometry (SEC/MS).

In an embodiment the presence of N-acetyl-D-fucosamine (D-FucNAc) and/or N-acetyl-D-quinovosamine (D-QuiNAc) residues is detected by Gas Chromatography-Mass Spectrometry (GC-MS). In an embodiment the presence of N-acetyl-D-fucosamine (D-FucNAc) and/or N-acetyl-D-quinovosamine (D-QuiNAc) residues is detected by Liquid Chromatography-Mass Spectrometry (LC-MS). In an embodiment the presence of N-acetyl-D-fucosamine (D-FucNAc) and/or N-acetyl-D-quinovosamine (D-QuiNAc) residues is detected by Capillary Electrophoresis-Mass Spectrometry (CE-MS). In an embodiment the presence of N-acetyl-D-fucosamine (D-FucNAc) and/or N-acetyl-D-quinovosamine (D-QuiNAc) residues is detected by Ion Mobility Spectrometry-Mass Spectrometry (IMS/MS). In an embodiment the presence of N-acetyl-D-fucosamine (D-FucNAc) and/or N-acetyl-D-quinovosamine (D-QuiNAc) residues is detected by Hydrophilic Interaction Liquid Chromatography-Mass Spectrometry (HILIC-LC/MS).

In an embodiment the invention relates to a method of detecting the presence of N-acetyl-D-fucosamine (D-FucNAc) and N-acetyl-D-quinovosamine (D-QuiNAc) residues in S. pneumoniae serotype 12F glycoconjugate, said method comprising the step of: a) preparing a S. pneumoniae serotype 12F glycoconjugate and b) detecting the presence of N-acetyl-D-fucosamine (D-FucNAc) and N-acetyl-D-quinovosamine (D-QuiNAc) residues in said glycoconjugate.

In an embodiment the presence of N-acetyl-D-fucosamine (D-FucNAc) and N-acetyl-D-quinovosamine (D-QuiNAc) residues is detected by NMR. In an embodiment the presence of N-acetyl-D-fucosamine (D-FucNAc) and N-acetyl-D-quinovosamine (D-QuiNAc) residues is detected by 2D NMR.

In an embodiment the presence of N-acetyl-D-fucosamine (D-FucNAc) and N-acetyl-D-quinovosamine (D-QuiNAc) residues is detected by Heteronuclear Single Quantum Coherence Spectroscopy (HSQC), Heteronuclear multiple-bond correlation spectroscopy (HMBC), Correlation spectroscopy (COSY), and/or Heteronuclear Single Quantum Coherence Spectroscopy-Total Correlation Spectroscopy (HSQC-TOCSY).

In a preferred embodiment, the presence of N-acetyl-D-fucosamine (D-FucNAc) and N-acetyl-D-quinovosamine (D-QuiNAc) residues is detected by 2D ¹H-¹³C HSQC NMR.

In an embodiment the presence of N-acetyl-D-fucosamine (D-FucNAc) and N-acetyl-D-quinovosamine (D-QuiNAc) residues is detected by Mass Spectrometry (MS). In an embodiment the presence of N-acetyl-D-fucosamine (D-FucNAc) and N-acetyl-D-quinovosamine (D-QuiNAc) residues is detected by Tandem Mass Spectrometry (MS/MS).

In an embodiment the presence of N-acetyl-D-fucosamine (D-FucNAc) and N-acetyl-D-quinovosamine (D-QuiNAc) residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the presence of N-acetyl-D-fucosamine (D-FucNAc) and N-acetyl-D-quinovosamine (D-QuiNAc) residues is detected by Size-Exclusion Chromatography combined with Mass Spectrometry (SEC/MS).

In an embodiment the presence of N-acetyl-D-fucosamine (D-FucNAc) and N-acetyl-D-quinovosamine (D-QuiNAc) residues is detected by Gas Chromatography-Mass Spectrometry (GC-MS). In an embodiment the presence of N-acetyl-D-fucosamine (D-FucNAc) and N-acetyl-D-quinovosamine (D-QuiNAc) residues is detected by Liquid Chromatography-Mass Spectrometry (LC-MS). In an embodiment the presence of N-acetyl-D-fucosamine (D-FucNAc) and N-acetyl-D-quinovosamine (D-QuiNAc) residues is detected by Capillary Electrophoresis-Mass Spectrometry (CE-MS). In an embodiment the presence of N-acetyl-D-fucosamine (D-FucNAc) and N-acetyl-D-quinovosamine (D-QuiNAc) residues is detected by Ion Mobility Spectrometry-Mass Spectrometry (IMS/MS). In an embodiment the presence of N-acetyl-D-fucosamine (D-FucNAc) and N-acetyl-D-quinovosamine (D-QuiNAc) residues is detected by Hydrophilic Interaction Liquid Chromatography-Mass Spectrometry (HILIC-LC/MS).

In an embodiment the invention relates to a method of detecting the presence of N-acetyl-D-fucosamine (D-FucNAc) residues in S. pneumoniae serotype 12F glycoconjugate, said method comprising the step of: a) preparing a S. pneumoniae serotype 12F glycoconjugate and b) detecting the presence of N-acetyl-D-fucosamine (D-FucNAc) residues in said glycoconjugate.

In an embodiment the presence of N-acetyl-D-fucosamine (D-FucNAc) residues is detected by NMR. In an embodiment the presence of N-acetyl-D-fucosamine (D-FucNAc) residues is detected by 2D NMR.

In an embodiment the presence of N-acetyl-D-fucosamine (D-FucNAc) residues is detected by Heteronuclear Single Quantum Coherence Spectroscopy (HSQC), Heteronuclear multiple-bond correlation spectroscopy (HMBC), Correlation spectroscopy (COSY), and/or Heteronuclear Single Quantum Coherence Spectroscopy-Total Correlation Spectroscopy (HSQC-TOCSY).

In a preferred embodiment, the presence of N-acetyl-D-fucosamine (D-FucNAc) residues is detected by 2D ¹H-¹³C HSQC NMR.

In an embodiment the presence of N-acetyl-D-fucosamine (D-FucNAc) residues is detected by Mass Spectrometry (MS). the presence of N-acetyl-D-fucosamine (D-FucNAc) residues is detected by Tandem Mass Spectrometry (MS/MS). In an embodiment the presence of N-acetyl-D-fucosamine (D-FucNAc) residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the presence of N-acetyl-D-fucosamine (D-FucNAc) residues is detected by Size-Exclusion Chromatography combined with Mass Spectrometry (SEC/MS).

In an embodiment the presence of N-acetyl-D-fucosamine (D-FucNAc) residues is detected by Gas Chromatography-Mass Spectrometry (GC-MS). In an embodiment the presence of N-acetyl-D-fucosamine (D-FucNAc) residues is detected by Liquid Chromatography-Mass Spectrometry (LC-MS). In an embodiment the presence of N-acetyl-D-fucosamine (D-FucNAc) residues is detected by Capillary Electrophoresis-Mass Spectrometry (CE-MS). In an embodiment the presence of N-acetyl-D-fucosamine (D-FucNAc) residues is detected by Ion Mobility Spectrometry-Mass Spectrometry (IMS/MS). In an embodiment the presence of N-acetyl-D-fucosamine (D-FucNAc) residues is detected by Hydrophilic Interaction Liquid Chromatography-Mass Spectrometry (HILIC-LC/MS).

In an embodiment the invention relates to a method of detecting the presence of N-acetyl-D-quinovosamine (D-QuiNAc) residues in S. pneumoniae serotype 12F glycoconjugate, said method comprising the step of: a) preparing a S. pneumoniae serotype 12F glycoconjugate and b) detecting the presence of N-acetyl-D-quinovosamine (D-QuiNAc) residues in said glycoconjugate.

In an embodiment the presence of N-acetyl-D-quinovosamine (D-QuiNAc) residues is detected by NMR. In an embodiment the presence of N-acetyl-D-quinovosamine (D-QuiNAc) residues is detected by 2D NMR.

In an embodiment the presence of N-acetyl-D-quinovosamine (D-QuiNAc) residues is detected by Heteronuclear Single Quantum Coherence Spectroscopy (HSQC), Heteronuclear multiple-bond correlation spectroscopy (HMBC), Correlation spectroscopy (COSY), and/or Heteronuclear Single Quantum Coherence Spectroscopy-Total Correlation Spectroscopy (HSQC-TOCSY).

In a preferred embodiment, the presence of N-acetyl-D-quinovosamine (D-QuiNAc) residues is detected by 2D ¹H-¹³C HSQC NMR.

In an embodiment the presence of N-acetyl-D-quinovosamine (D-QuiNAc) residues is detected by Mass Spectrometry (MS). In an embodiment the presence of N-acetyl-D-quinovosamine (D-QuiNAc) residues is detected by Tandem Mass Spectrometry (MS/MS). In an embodiment the presence of N-acetyl-D-quinovosamine (D-QuiNAc) residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the presence of N-acetyl-D-quinovosamine (D-QuiNAc) residues is detected by Size-Exclusion Chromatography combined with Mass Spectrometry (SEC/MS).

In an embodiment the presence of N-acetyl-D-quinovosamine (D-QuiNAc) residues is detected by Gas Chromatography-Mass Spectrometry (GC-MS). In an embodiment the presence of N-acetyl-D-quinovosamine (D-QuiNAc) residues is detected by Liquid Chromatography-Mass Spectrometry (LC-MS). In an embodiment the presence of N-acetyl-D-quinovosamine (D-QuiNAc) residues is detected by Capillary Electrophoresis-Mass Spectrometry (CE-MS). In an embodiment the presence of N-acetyl-D-quinovosamine (D-QuiNAc) residues is detected by Ion Mobility Spectrometry-Mass Spectrometry (IMS/MS). In an embodiment the presence of N-acetyl-D-quinovosamine (D-QuiNAc) residues is detected by Hydrophilic Interaction Liquid Chromatography-Mass Spectrometry (HILIC-LC/MS).

### 7. The Invention Also Provides the Following Embodiments as Defined in the Following Numbered Paragraphs 1 to 296

1. An isolated polysaccharide with the following repeating unit:

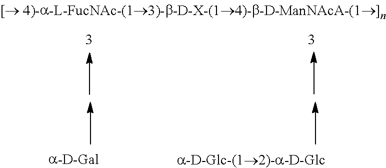

where n represents the number of repeating units and where X represents either N-acetylgalactosamine or 4-keto-N-acetyl-quinovosamine.

2. The isolated polysaccharide of paragraph 1 where said isolated polysaccharide comprises between about 99.9 to about 50 N-acetylgalactosamine residues and about 0.1 to about 50 4-keto-N-acetyl-quinovosamine residues in every 100 saccharide repeat units of the polysaccharide.

3. The isolated polysaccharide of paragraph 1 where said isolated polysaccharide comprises between about 99.8 to about 50 N-acetylgalactosamine residues and about 0.2 to about 50 4-keto-N-acetyl-quinovosamine residues in every 100 saccharide repeat units of the polysaccharide.

4. The isolated polysaccharide of paragraph 1 where said polysaccharide comprises between about 99.1 to about 50 N-acetylgalactosamine residues and about 0.9 to about 50 4-keto-N-acetyl-quinovosamine residues in every 100 saccharide repeat units of the polysaccharide.

5. The isolated polysaccharide of paragraph 1 where said isolated polysaccharide comprises between about 99 to about 50 N-acetylgalactosamine residues and about 1 to about 50 4-keto-N-acetyl-quinovosamine residues in every 100 saccharide repeat units of the polysaccharide.

6. The isolated polysaccharide of paragraph 1 where said isolated polysaccharide comprises between about 95 to about 50 N-acetylgalactosamine residues and about 5 to about 50 4-keto-N-acetyl-quinovosamine residues in every 100 saccharide repeat units of the polysaccharide.

7. The isolated polysaccharide of paragraph 1 where said isolated polysaccharide comprises between about 90 to about 50 N-acetylgalactosamine residues and about 10 to about 50 4-keto-N-acetyl-quinovosamine residues in every 100 saccharide repeat units of the polysaccharide.

8. The isolated polysaccharide of paragraph 1 where said isolated polysaccharide comprises between about 99.9 to about 55 N-acetylgalactosamine residues and about 0.1 to about 45 4-keto-N-acetyl-quinovosamine residues in every 100 saccharide repeat units of the polysaccharide.

9. The isolated polysaccharide of paragraph 1 where said isolated polysaccharide comprises between about 99.1 to about 55 N-acetylgalactosamine residues and about 0.9 to about 45 4-keto-N-acetyl-quinovosamine residues in every 100 saccharide repeat units of the polysaccharide.

10. The isolated polysaccharide of paragraph 1 where said isolated polysaccharide comprises between about 90 to about 55 N-acetylgalactosamine residues and about 10 to about 45 4-keto-N-acetyl-quinovosamine residues in every 100 saccharide repeat units of the polysaccharide.

11. The isolated polysaccharide of paragraph 1 where said isolated polysaccharide comprises between about 99.9 to about 75 N-acetylgalactosamine residues and about 0.1 to about 25 4-keto-N-acetyl-quinovosamine residues in every 100 saccharide repeat units of the polysaccharide.

12. The isolated polysaccharide of paragraph 1 where said isolated polysaccharide comprises between about 99 to about 75 N-acetylgalactosamine residues and about 1 to about 25 4-keto-N-acetyl-quinovosamine residues in every 100 saccharide repeat units of the polysaccharide.

13. The isolated polysaccharide of paragraph 1 where said isolated polysaccharide comprises between about 90 to about 75 N-acetylgalactosamine residues and about 10 to about 25 4-keto-N-acetyl-quinovosamine residues in every 100 saccharide repeat units of the polysaccharide.

14. The isolated polysaccharide of paragraph 1 where said isolated polysaccharide comprises between about 99.9 to about 99.5 N-acetylgalactosamine residues and about 0.1 to about 0.5 4-keto-N-acetyl-quinovosamine residues in every 100 saccharide repeat units of the polysaccharide.

15. The isolated polysaccharide of paragraph 1 where said isolated polysaccharide comprises between about 99.9 to about 99 N-acetylgalactosamine residues and about 0.1 to about 1 4-keto-N-acetyl-quinovosamine residues in every 100 saccharide repeat units of the polysaccharide.

16. The isolated polysaccharide of paragraph 1 where said isolated polysaccharide comprises between about 99.9 to about 98 N-acetylgalactosamine residues and about 0.1 to about 2 4-keto-N-acetyl-quinovosamine residues in every 100 saccharide repeat units of the polysaccharide.

17. The isolated polysaccharide of paragraph 1 where said isolated polysaccharide comprises between about 99.8 to about 99.5 N-acetylgalactosamine residues and about 0.2 to about 0.5 4-keto-N-acetyl-quinovosamine residues in every 100 saccharide repeat units of the polysaccharide.

18. The isolated polysaccharide of paragraph 1 where said isolated polysaccharide comprises between about 99.8 to about 99 N-acetylgalactosamine residues and about 0.2 to about 1 4-keto-N-acetyl-quinovosamine residues in every 100 saccharide repeat units of the polysaccharide.

19. The isolated polysaccharide of paragraph 1 where said isolated polysaccharide comprises about 99.9 N-acetylgalactosamine residues and about 0.1 4-keto-N-acetyl-quinovosamine residues in every 100 saccharide repeat units of the polysaccharide.

20. The isolated polysaccharide of paragraph 1 where said isolated polysaccharide comprises about 99.8 N-acetylgalactosamine residues and about 0.2 4-keto-N-acetyl-quinovosamine residues in every 100 saccharide repeat units of the polysaccharide.

21. The isolated polysaccharide of paragraph 1 where said isolated polysaccharide comprises about 80 N-acetylgalactosamine residues and about 20 4-keto-N-acetyl-quinovosamine residues in every 100 saccharide repeat units of the polysaccharide.

22. The isolated polysaccharide of paragraph 1 where said isolated polysaccharide comprises about 75 N-acetylgalactosamine residues and about 25 4-keto-N-acetyl-quinovosamine residues in every 100 saccharide repeat units of the polysaccharide.

23. An isolated S. pneumoniae serotype 12F capsular polysaccharide comprising between about 99.9 to about 50 N-acetylgalactosamine residues and about 0.1 to about 50 4-keto-N-acetyl-quinovosamine residues in every 100 saccharide repeat units of the polysaccharide.

24. An isolated S. pneumoniae serotype 12F capsular polysaccharide comprising between about 99.9 to about 55 N-acetylgalactosamine residues and about 0.1 to about 45 4-keto-N-acetyl-quinovosamine residues in every 100 saccharide repeat units of the polysaccharide.

25. An isolated S. pneumoniae serotype 12F capsular polysaccharide comprising between about 99.9 to about 75 N-acetylgalactosamine residues and about 0.1 to about 25 4-keto-N-acetyl-quinovosamine residues in every 100 saccharide repeat units of the polysaccharide.

26. An isolated S. pneumoniae serotype 12F capsular polysaccharide comprising between about 99 to about 75 N-acetylgalactosamine residues and about 1 to about 25 4-keto-N-acetyl-quinovosamine residues in every 100 saccharide repeat units of the polysaccharide.

27. An isolated S. pneumoniae serotype 12F capsular polysaccharide comprising between about 95 to about 50 N-acetylgalactosamine residues and about 5 to about 50 4-keto-N-acetyl-quinovosamine residues in every 100 saccharide repeat units of the polysaccharide.

28. An isolated S. pneumoniae serotype 12F capsular polysaccharide comprising between about 95 to about 55 N-acetylgalactosamine residues and about 5 to about 45 4-keto-N-acetyl-quinovosamine residues in every 100 saccharide repeat units of the polysaccharide.

29. An isolated S. pneumoniae serotype 12F capsular polysaccharide comprising between about 95 to about 75 N-acetylgalactosamine residues and about 5 to about 25 4-keto-N-acetyl-quinovosamine residues in every 100 saccharide repeat units of the polysaccharide.

30. An isolated S. pneumoniae serotype 12F capsular polysaccharide comprising between about 90 to about 50 N-acetylgalactosamine residues and about 10 to about 50 4-keto-N-acetyl-quinovosamine residues in every 100 saccharide repeat units of the polysaccharide.

31. An isolated S. pneumoniae serotype 12F capsular polysaccharide comprising between about 90 to about 55 N-acetylgalactosamine residues and about 10 to about 45 4-keto-N-acetyl-quinovosamine residues in every 100 saccharide repeat units of the polysaccharide.

32. An isolated S. pneumoniae serotype 12F capsular polysaccharide comprising between about 90 to about 75 N-acetylgalactosamine residues and about 10 to about 25 4-keto-N-acetyl-quinovosamine residues in every 100 saccharide repeat units of the polysaccharide.

33. An isolated S. pneumoniae serotype 12F capsular polysaccharide comprising between about 99.9 to about 99.5 N-acetylgalactosamine residues and about 0.1 to about 0.5 4-keto-N-acetyl-quinovosamine residues in every 100 saccharide repeat units of the polysaccharide.

34. An isolated S. pneumoniae serotype 12F capsular polysaccharide comprising between about 99.9 to about 99 N-acetylgalactosamine residues and about 0.1 to about 1 4-keto-N-acetyl-quinovosamine residues in every 100 saccharide repeat units of the polysaccharide.

35. An isolated S. pneumoniae serotype 12F capsular polysaccharide comprising between about 99.8 to about 99.5 N-acetylgalactosamine residues and about 0.2 to about 0.5 4-keto-N-acetyl-quinovosamine residues in every 100 saccharide repeat units of the polysaccharide.

36. The isolated polysaccharide of any one of paragraphs 1-35 wherein said isolated polysaccharide has between 10 and 5,000 repeating units.

37. The isolated polysaccharide of any one of paragraphs 1-35 wherein said isolated polysaccharide has between 50 and 4,500 repeating units.

38. The isolated polysaccharide of any one of paragraphs 1-35 wherein said wherein said isolated polysaccharide has between 100 and 4,500 repeating units.

39. The isolated polysaccharide of any one of paragraphs 1-35 wherein said isolated polysaccharide has between 150 and 2,000 repeating units.

40. The isolated polysaccharide of any one of paragraphs 1-39 wherein said isolated polysaccharide has a weight average molecular weight between 5 kDa and 5000 kDa.

41. The isolated polysaccharide of any one of paragraphs 1-39 wherein said isolated polysaccharide has a weight average molecular weight between 5 kDa and 2000 kDa.

42. The isolated polysaccharide of any one of paragraphs 1-39 wherein said isolated polysaccharide has a weight average molecular weight between 50 kDa and 1000 kDa.

43. The isolated polysaccharide of any one of paragraphs 1-39 wherein said isolated polysaccharide has a weight average molecular weight between 50 kDa and 300 kDa.

44. The isolated polysaccharide of any one of paragraphs 1-39 wherein said isolated polysaccharide has a weight average molecular weight between 100 kDa and 500 kDa.

45. The isolated polysaccharide of any one of paragraphs 1-39 wherein said isolated polysaccharide has a weight average molecular weight between 100 kDa and 300 kDa.

46. The isolated polysaccharide of any one of paragraphs 1-39 wherein said isolated polysaccharide has a weight average molecular weight between 200 kDa and 1000 kDa.

47. The isolated polysaccharide of any one of paragraphs 1-39 wherein said isolated polysaccharide has a weight average molecular weight between 200 kDa and 500 kDa.

48. The isolated polysaccharide of any one of paragraphs 1-39 wherein said isolated capsular polysaccharide has a weight average molecular weight between 300 kDa and 400 kDa.

49. The isolated polysaccharide of any one of paragraphs 1-39 wherein said isolated capsular polysaccharide has a weight average molecular weight between 100 kDa and 500 kDa.

50. A S. pneumoniae serotype 12F glycoconjugate prepared by a process comprising the step of: a) reacting the isolated polysaccharide of any one of paragraphs 1-49 with an activating agent to produce an activated saccharide; and b) reacting the activated saccharide with a carrier protein.

51. A S. pneumoniae serotype 12F glycoconjugate comprising a serotype 12F capsular polysaccharide comprising between about 0.05 to about 25 N-acetyl-D-fucosamine (D-FucNAc) residues in every 100 saccharide repeat units of the polysaccharide.

52. A S. pneumoniae serotype 12F glycoconjugate comprising a serotype 12F capsular polysaccharide comprising between about 0.05 to about 22.5 N-acetyl-D-fucosamine (D-FucNAc) residues in every 100 saccharide repeat units of the polysaccharide.

53. A S. pneumoniae serotype 12F glycoconjugate comprising a serotype 12F capsular polysaccharide comprising between about 0.05 to about 15 N-acetyl-D-fucosamine (D-FucNAc) residues in every 100 saccharide repeat units of the polysaccharide.

54. A S. pneumoniae serotype 12F glycoconjugate comprising a serotype 12F capsular polysaccharide comprising between about 0.05 to about 12.5 N-acetyl-D-fucosamine (D-FucNAc) residues in every 100 saccharide repeat units of the polysaccharide.

55. A S. pneumoniae serotype 12F glycoconjugate comprising a serotype 12F capsular polysaccharide comprising between about 0.1 to about 25 N-acetyl-D-fucosamine (D-FucNAc) residues in every 100 saccharide repeat units of the polysaccharide.

56. A S. pneumoniae serotype 12F glycoconjugate comprising a serotype 12F capsular polysaccharide comprising between about 0.1 to about 22.5 N-acetyl-D-fucosamine (D-FucNAc) residues in every 100 saccharide repeat units of the polysaccharide.

57. A S. pneumoniae serotype 12F glycoconjugate comprising a serotype 12F capsular polysaccharide comprising between about 0.1 to about 15 N-acetyl-D-fucosamine (D-FucNAc) residues in every 100 saccharide repeat units of the polysaccharide.

58. A S. pneumoniae serotype 12F glycoconjugate comprising a serotype 12F capsular polysaccharide comprising between about 0.1 to about 12.5 N-acetyl-D-fucosamine (D-FucNAc) residues in every 100 saccharide repeat units of the polysaccharide.

59. A S. pneumoniae serotype 12F glycoconjugate comprising a serotype 12F capsular polysaccharide comprising between about 0.5 to about 25 N-acetyl-D-fucosamine (D-FucNAc) residues in every 100 saccharide repeat units of the polysaccharide.

60. A S. pneumoniae serotype 12F glycoconjugate comprising a serotype 12F capsular polysaccharide comprising between about 0.5 to about 22.5 N-acetyl-D-fucosamine (D-FucNAc) residues in every 100 saccharide repeat units of the polysaccharide.

61. A S. pneumoniae serotype 12F glycoconjugate comprising a serotype 12F capsular polysaccharide comprising between about 0.5 to about 15 N-acetyl-D-fucosamine (D-FucNAc) residues in every 100 saccharide repeat units of the polysaccharide.

62. A S. pneumoniae serotype 12F glycoconjugate comprising a serotype 12F capsular polysaccharide comprising between about 0.5 to about 12.5 N-acetyl-D-fucosamine (D-FucNAc) residues in every 100 saccharide repeat units of the polysaccharide.

63. A S. pneumoniae serotype 12F glycoconjugate comprising a serotype 12F capsular polysaccharide comprising between about 1 to about 25 N-acetyl-D-fucosamine (D-FucNAc) residues in every 100 saccharide repeat units of the polysaccharide.

64. A S. pneumoniae serotype 12F glycoconjugate comprising a serotype 12F capsular polysaccharide comprising between about 1 to about 22.5 N-acetyl-D-fucosamine (D-FucNAc) residues in every 100 saccharide repeat units of the polysaccharide.

65. A S. pneumoniae serotype 12F glycoconjugate comprising a serotype 12F capsular polysaccharide comprising between about 1 to about 15 N-acetyl-D-fucosamine (D-FucNAc) residues in every 100 saccharide repeat units of the polysaccharide.

66. A S. pneumoniae serotype 12F glycoconjugate comprising a serotype 12F capsular polysaccharide comprising between about 1 to about 12.5 N-acetyl-D-fucosamine (D-FucNAc) residues in every 100 saccharide repeat units of the polysaccharide.

67. A S. pneumoniae serotype 12F glycoconjugate comprising a serotype 12F capsular polysaccharide comprising between about 2 to about 25 N-acetyl-D-fucosamine (D-FucNAc) residues in every 100 saccharide repeat units of the polysaccharide.

68. A S. pneumoniae serotype 12F glycoconjugate comprising a serotype 12F capsular polysaccharide comprising between about 2 to about 22.5 N-acetyl-D-fucosamine (D-FucNAc) residues in every 100 saccharide repeat units of the polysaccharide.

69. A S. pneumoniae serotype 12F glycoconjugate comprising a serotype 12F capsular polysaccharide comprising between about 2 to about 15 N-acetyl-D-fucosamine (D-FucNAc) residues in every 100 saccharide repeat units of the polysaccharide.

70. A S. pneumoniae serotype 12F glycoconjugate comprising a serotype 12F capsular polysaccharide comprising between about 2 to about 12.5 N-acetyl-D-fucosamine (D-FucNAc) residues in every 100 saccharide repeat units of the polysaccharide.

71. A S. pneumoniae serotype 12F glycoconjugate comprising a serotype 12F capsular polysaccharide comprising between about 3 to about 25 N-acetyl-D-fucosamine (D-FucNAc) residues in every 100 saccharide repeat units of the polysaccharide.

72. A S. pneumoniae serotype 12F glycoconjugate comprising a serotype 12F capsular polysaccharide comprising between about 3 to about 22.5 N-acetyl-D-fucosamine (D-FucNAc) residues in every 100 saccharide repeat units of the polysaccharide.

73. A S. pneumoniae serotype 12F glycoconjugate comprising a serotype 12F capsular polysaccharide comprising between about 3 to about 15 N-acetyl-D-fucosamine (D-FucNAc) residues in every 100 saccharide repeat units of the polysaccharide.

74. A S. pneumoniae serotype 12F glycoconjugate comprising a serotype 12F capsular polysaccharide comprising between about 3 to about 12.5 N-acetyl-D-fucosamine (D-FucNAc) residues in every 100 saccharide repeat units of the polysaccharide.

75. A S. pneumoniae serotype 12F glycoconjugate comprising a serotype 12F capsular polysaccharide comprising between about 4 to about 25 N-acetyl-D-fucosamine (D-FucNAc) residues in every 100 saccharide repeat units of the polysaccharide.

76. A S. pneumoniae serotype 12F glycoconjugate comprising a serotype 12F capsular polysaccharide comprising between about 4 to about 22.5 N-acetyl-D-fucosamine (D-FucNAc) residues in every 100 saccharide repeat units of the polysaccharide.

77. A S. pneumoniae serotype 12F glycoconjugate comprising a serotype 12F capsular polysaccharide comprising between about 4 to about 15 N-acetyl-D-fucosamine (D-FucNAc) residues in every 100 saccharide repeat units of the polysaccharide.

78. A S. pneumoniae serotype 12F glycoconjugate comprising a serotype 12F capsular polysaccharide comprising between about 4 to about 12.5 N-acetyl-D-fucosamine (D-FucNAc) residues in every 100 saccharide repeat units of the polysaccharide.

79. A S. pneumoniae serotype 12F glycoconjugate comprising a serotype 12F capsular polysaccharide comprising between about 5 to about 25 N-acetyl-D-fucosamine (D-FucNAc) residues in every 100 saccharide repeat units of the polysaccharide.

80. A S. pneumoniae serotype 12F glycoconjugate comprising a serotype 12F capsular polysaccharide comprising between about 5 to about 22.5 N-acetyl-D-fucosamine (D-FucNAc) residues in every 100 saccharide repeat units of the polysaccharide.

81. A S. pneumoniae serotype 12F glycoconjugate comprising a serotype 12F capsular polysaccharide comprising between about 5 to about 15 N-acetyl-D-fucosamine (D-FucNAc) residues in every 100 saccharide repeat units of the polysaccharide.

82. A S. pneumoniae serotype 12F glycoconjugate comprising a serotype 12F capsular polysaccharide comprising between about 5 to about 12.5 N-acetyl-D-fucosamine (D-FucNAc) residues in every 100 saccharide repeat units of the polysaccharide.

83. A S. pneumoniae serotype 12F glycoconjugate comprising a serotype 12F capsular polysaccharide comprising between about 10 to about 25 N-acetyl-D-fucosamine (D-FucNAc) residues in every 100 saccharide repeat units of the polysaccharide.

84. A S. pneumoniae serotype 12F glycoconjugate comprising a serotype 12F capsular polysaccharide comprising between about 10 to about 22.5 N-acetyl-D-fucosamine (D-FucNAc) residues in every 100 saccharide repeat units of the polysaccharide.

85. A S. pneumoniae serotype 12F glycoconjugate comprising a serotype 12F capsular polysaccharide comprising between about 10 to about 15 N-acetyl-D-fucosamine (D-FucNAc) residues in every 100 saccharide repeat units of the polysaccharide.

86. A S. pneumoniae serotype 12F glycoconjugate comprising a serotype 12F capsular polysaccharide comprising between about 10 to about 12.5 N-acetyl-D-fucosamine (D-FucNAc) residues in every 100 saccharide repeat units of the polysaccharide.

87. A S. pneumoniae serotype 12F glycoconjugate comprising a serotype 12F capsular polysaccharide comprising between about 0.05 to about 25 N-acetyl-D-quinovosamine (D-QuiNAc) residues in every 100 saccharide repeat units of the polysaccharide.

88. A S. pneumoniae serotype 12F glycoconjugate comprising a serotype 12F capsular polysaccharide comprising between about 0.05 to about 22.5 N-acetyl-D-quinovosamine (D-QuiNAc) residues in every 100 saccharide repeat units of the polysaccharide.

89. A S. pneumoniae serotype 12F glycoconjugate comprising a serotype 12F capsular polysaccharide comprising between about 0.05 to about 15 N-acetyl-D-quinovosamine (D-QuiNAc) residues in every 100 saccharide repeat units of the polysaccharide.

90. A S. pneumoniae serotype 12F glycoconjugate comprising a serotype 12F capsular polysaccharide comprising between about 0.05 to about 12.5 N-acetyl-D-quinovosamine (D-QuiNAc) residues in every 100 saccharide repeat units of the polysaccharide.

91. A S. pneumoniae serotype 12F glycoconjugate comprising a serotype 12F capsular polysaccharide comprising between about 0.1 to about 25 N-acetyl-D-quinovosamine (D-QuiNAc) residues in every 100 saccharide repeat units of the polysaccharide.

92. A S. pneumoniae serotype 12F glycoconjugate comprising a serotype 12F capsular polysaccharide comprising between about 0.1 to about 22.5 N-acetyl-D-quinovosamine (D-QuiNAc) residues in every 100 saccharide repeat units of the polysaccharide.

93. A S. pneumoniae serotype 12F glycoconjugate comprising a serotype 12F capsular polysaccharide comprising between about 0.1 to about 15 N-acetyl-D-quinovosamine (D-QuiNAc) residues in every 100 saccharide repeat units of the polysaccharide.

94. A S. pneumoniae serotype 12F glycoconjugate comprising a serotype 12F capsular polysaccharide comprising between about 0.1 to about 12.5 N-acetyl-D-quinovosamine (D-QuiNAc) residues in every 100 saccharide repeat units of the polysaccharide.

95. A S. pneumoniae serotype 12F glycoconjugate comprising a serotype 12F capsular polysaccharide comprising between about 0.5 to about 25 N-acetyl-D-quinovosamine (D-QuiNAc) residues in every 100 saccharide repeat units of the polysaccharide.

96. A S. pneumoniae serotype 12F glycoconjugate comprising a serotype 12F capsular polysaccharide comprising between about 0.5 to about 22.5 N-acetyl-D-quinovosamine (D-QuiNAc) residues in every 100 saccharide repeat units of the polysaccharide.

97. A S. pneumoniae serotype 12F glycoconjugate comprising a serotype 12F capsular polysaccharide comprising between about 0.5 to about 15 N-acetyl-D-quinovosamine (D-QuiNAc) residues in every 100 saccharide repeat units of the polysaccharide.

98. A S. pneumoniae serotype 12F glycoconjugate comprising a serotype 12F capsular polysaccharide comprising between about 0.5 to about 12.5 N-acetyl-D-quinovosamine (D-QuiNAc) residues in every 100 saccharide repeat units of the polysaccharide.

99. A S. pneumoniae serotype 12F glycoconjugate comprising a serotype 12F capsular polysaccharide comprising between about 1 to about 25 N-acetyl-D-quinovosamine (D-QuiNAc) residues in every 100 saccharide repeat units of the polysaccharide.

100. A S. pneumoniae serotype 12F glycoconjugate comprising a serotype 12F capsular polysaccharide comprising between about 1 to about 22.5 N-acetyl-D-quinovosamine (D-QuiNAc) residues in every 100 saccharide repeat units of the polysaccharide.

101. A S. pneumoniae serotype 12F glycoconjugate comprising a serotype 12F capsular polysaccharide comprising between about 1 to about 15 N-acetyl-D-quinovosamine (D-QuiNAc) residues in every 100 saccharide repeat units of the polysaccharide.

102. A S. pneumoniae serotype 12F glycoconjugate comprising a serotype 12F capsular polysaccharide comprising between about 1 to about 12.5 N-acetyl-D-quinovosamine (D-QuiNAc) residues in every 100 saccharide repeat units of the polysaccharide.

103. A S. pneumoniae serotype 12F glycoconjugate comprising a serotype 12F capsular polysaccharide comprising between about 2 to about 25 N-acetyl-D-quinovosamine (D-QuiNAc) residues in every 100 saccharide repeat units of the polysaccharide.

104. A S. pneumoniae serotype 12F glycoconjugate comprising a serotype 12F capsular polysaccharide comprising between about 2 to about 22.5 N-acetyl-D-quinovosamine (D-QuiNAc) residues in every 100 saccharide repeat units of the polysaccharide.

105. A S. pneumoniae serotype 12F glycoconjugate comprising a serotype 12F capsular polysaccharide comprising between about 2 to about 15 N-acetyl-D-quinovosamine (D-QuiNAc) residues in every 100 saccharide repeat units of the polysaccharide.

106. A S. pneumoniae serotype 12F glycoconjugate comprising a serotype 12F capsular polysaccharide comprising between about 2 to about 12.5 N-acetyl-D-quinovosamine (D-QuiNAc) residues in every 100 saccharide repeat units of the polysaccharide.

107. A S. pneumoniae serotype 12F glycoconjugate comprising a serotype 12F capsular polysaccharide comprising between about 3 to about 25 N-acetyl-D-quinovosamine (D-QuiNAc) residues in every 100 saccharide repeat units of the polysaccharide.

108. A serotype 12F glycoconjugate comprising a serotype 12F capsular polysaccharide comprising between about 3 to about 22.5 N-acetyl-D-quinovosamine (D-QuiNAc) residues in every 100 saccharide repeat units of the polysaccharide.

109. A S. pneumoniae serotype 12F glycoconjugate comprising a serotype 12F capsular polysaccharide comprising between about 3 to about 15 N-acetyl-D-quinovosamine (D-QuiNAc) residues in every 100 saccharide repeat units of the polysaccharide.

110. A S. pneumoniae serotype 12F glycoconjugate comprising a serotype 12F capsular polysaccharide comprising between about 3 to about 12.5 N-acetyl-D-quinovosamine (D-QuiNAc) residues in every 100 saccharide repeat units of the polysaccharide.

111. A S. pneumoniae serotype 12F glycoconjugate comprising a serotype 12F capsular polysaccharide comprising between about 4 to about 25 N-acetyl-D-quinovosamine (D-QuiNAc) residues in every 100 saccharide repeat units of the polysaccharide.

112. A S. pneumoniae serotype 12F glycoconjugate comprising a serotype 12F capsular polysaccharide comprising between about 4 to about 22.5 N-acetyl-D-quinovosamine (D-QuiNAc) residues in every 100 saccharide repeat units of the polysaccharide.

113. A S. pneumoniae serotype 12F glycoconjugate comprising a serotype 12F capsular polysaccharide comprising between about 4 to about 15 N-acetyl-D-quinovosamine (D-QuiNAc) residues in every 100 saccharide repeat units of the polysaccharide.

114. A S. pneumoniae serotype 12F glycoconjugate comprising a serotype 12F capsular polysaccharide comprising between about 4 to about 12.5 N-acetyl-D-quinovosamine (D-QuiNAc) residues in every 100 saccharide repeat units of the polysaccharide.

115. A S. pneumoniae serotype 12F glycoconjugate comprising a serotype 12F capsular polysaccharide comprising between about 5 to about 25 N-acetyl-D-quinovosamine (D-QuiNAc) residues in every 100 saccharide repeat units of the polysaccharide.

116. A S. pneumoniae serotype 12F glycoconjugate comprising a serotype 12F capsular polysaccharide comprising between about 5 to about 22.5 N-acetyl-D-quinovosamine (D-QuiNAc) residues in every 100 saccharide repeat units of the polysaccharide.

117. A S. pneumoniae serotype 12F glycoconjugate comprising a serotype 12F capsular polysaccharide comprising between about 5 to about 15 N-acetyl-D-quinovosamine (D-QuiNAc) residues in every 100 saccharide repeat units of the polysaccharide.

118. A S. pneumoniae serotype 12F glycoconjugate comprising a serotype 12F capsular polysaccharide comprising between about 5 to about 12.5 N-acetyl-D-quinovosamine (D-QuiNAc) residues in every 100 saccharide repeat units of the polysaccharide.

119. A S. pneumoniae serotype 12F glycoconjugate comprising a serotype 12F capsular polysaccharide comprising between about 10 to about 25 N-acetyl-D-quinovosamine (D-QuiNAc) residues in every 100 saccharide repeat units of the polysaccharide.

120. A S. pneumoniae serotype 12F glycoconjugate comprising a serotype 12F capsular polysaccharide comprising between about 10 to about 22.5 N-acetyl-D-quinovosamine (D-QuiNAc) residues in every 100 saccharide repeat units of the polysaccharide.

121. A S. pneumoniae serotype 12F glycoconjugate comprising a serotype 12F capsular polysaccharide comprising between about 10 to about 15 N-acetyl-D-quinovosamine (D-QuiNAc) residues in every 100 saccharide repeat units of the polysaccharide.

122. A S. pneumoniae serotype 12F glycoconjugate comprising a serotype 12F capsular polysaccharide comprising between about 10 to about 12.5 N-acetyl-D-quinovosamine (D-QuiNAc) residues in every 100 saccharide repeat units of the polysaccharide.

123. A S. pneumoniae serotype 12F glycoconjugate comprising a serotype 12F capsular polysaccharide comprising between about 0.05 to about 25 N-acetyl-D-fucosamine (D-FucNAc) residues and between about 0.05 to about 25 N-acetyl-D-quinovosamine (D-QuiNAc) in every 100 saccharide repeat units of the polysaccharide.

124. A S. pneumoniae serotype 12F glycoconjugate comprising a serotype 12F capsular polysaccharide comprising between about 0.05 to about 22.5 N-acetyl-D-fucosamine (D-FucNAc) residues and between about 0.05 to about 22.5 N-acetyl-D-quinovosamine (D-QuiNAc) in every 100 saccharide repeat units of the polysaccharide.

125. A S. pneumoniae serotype 12F glycoconjugate comprising a serotype 12F capsular polysaccharide comprising between about 0.05 to about 15 N-acetyl-D-fucosamine (D-FucNAc) residues and between about 0.05 to about 15 N-acetyl-D-quinovosamine (D-QuiNAc) in every 100 saccharide repeat units of the polysaccharide.

126. A S. pneumoniae serotype 12F glycoconjugate comprising a serotype 12F capsular polysaccharide comprising between about 0.1 to about 12.5 N-acetyl-D-fucosamine (D-FucNAc) residues and between about 0.1 to about 12.5 N-acetyl-D-quinovosamine (D-QuiNAc) in every 100 saccharide repeat units of the polysaccharide.

127. A S. pneumoniae serotype 12F glycoconjugate comprising a serotype 12F capsular polysaccharide comprising between about 0.1 to about 25 N-acetyl-D-fucosamine (D-FucNAc) residues and between about 0.1 to about 25 N-acetyl-D-quinovosamine (D-QuiNAc) in every 100 saccharide repeat units of the polysaccharide.

128. A S. pneumoniae serotype 12F glycoconjugate comprising a serotype 12F capsular polysaccharide comprising between about 0.1 to about 22.5 N-acetyl-D-fucosamine (D-FucNAc) residues and between about 0.1 to about 22.5 N-acetyl-D-quinovosamine (D-QuiNAc) in every 100 saccharide repeat units of the polysaccharide.

129. A S. pneumoniae serotype 12F glycoconjugate comprising a serotype 12F capsular polysaccharide comprising between about 0.1 to about 15 N-acetyl-D-fucosamine (D-FucNAc) residues and between about 0.1 to about 15 N-acetyl-D-quinovosamine (D-QuiNAc) in every 100 saccharide repeat units of the polysaccharide.

130. A S. pneumoniae serotype 12F glycoconjugate comprising a serotype 12F capsular polysaccharide comprising between about 0.1 to about 12.5 N-acetyl-D-fucosamine (D-FucNAc) residues and between about 0.1 to about 12.5 N-acetyl-D-quinovosamine (D-QuiNAc) in every 100 saccharide repeat units of the polysaccharide.

131. A S. pneumoniae serotype 12F glycoconjugate comprising a serotype 12F capsular polysaccharide comprising between about 0.5 to about 25 N-acetyl-D-fucosamine (D-FucNAc) residues and between about 0.5 to about 25 N-acetyl-D-quinovosamine (D-QuiNAc) in every 100 saccharide repeat units of the polysaccharide.

132. A S. pneumoniae serotype 12F glycoconjugate comprising a serotype 12F capsular polysaccharide comprising between about 0.05 to about 22.5 N-acetyl-D-fucosamine (D-FucNAc) residues and between about 0.5 to about 22.5 N-acetyl-D-quinovosamine (D-QuiNAc) in every 100 saccharide repeat units of the polysaccharide.

133. A S. pneumoniae serotype 12F glycoconjugate comprising a serotype 12F capsular polysaccharide comprising between about 0.5 to about 15 N-acetyl-D-fucosamine (D-FucNAc) residues and between about 0.5 to about 15 N-acetyl-D-quinovosamine (D-QuiNAc) in every 100 saccharide repeat units of the polysaccharide.

134. A S. pneumoniae serotype 12F glycoconjugate comprising a serotype 12F capsular polysaccharide comprising between about 0.5 to about 12.5 N-acetyl-D-fucosamine (D-FucNAc) residues and between about 0.5 to about 12.5 N-acetyl-D-quinovosamine (D-QuiNAc) in every 100 saccharide repeat units of the polysaccharide.

135. A S. pneumoniae serotype 12F glycoconjugate comprising a serotype 12F capsular polysaccharide comprising between about 1 to about 25 N-acetyl-D-fucosamine (D-FucNAc) residues and between about 1 to about 25 N-acetyl-D-quinovosamine (D-QuiNAc) in every 100 saccharide repeat units of the polysaccharide.

136. A S. pneumoniae serotype 12F glycoconjugate comprising a serotype 12F capsular polysaccharide comprising between about 1 to about 22.5 N-acetyl-D-fucosamine (D-FucNAc) residues and between about 1 to about 22.5 N-acetyl-D-quinovosamine (D-QuiNAc) in every 100 saccharide repeat units of the polysaccharide.

137. A S. pneumoniae serotype 12F glycoconjugate comprising a serotype 12F capsular polysaccharide comprising between about 1 to about 15 N-acetyl-D-fucosamine (D-FucNAc) residues and between about 1 to about 15 N-acetyl-D-quinovosamine (D-QuiNAc) in every 100 saccharide repeat units of the polysaccharide.

138. A S. pneumoniae serotype 12F glycoconjugate comprising a serotype 12F capsular polysaccharide comprising between about 1 to about 12.5 N-acetyl-D-fucosamine (D-FucNAc) residues and between about 1 to about 12.5 N-acetyl-D-quinovosamine (D-QuiNAc) in every 100 saccharide repeat units of the polysaccharide.

139. A S. pneumoniae serotype 12F glycoconjugate comprising a serotype 12F capsular polysaccharide comprising between about 2 to about 25 N-acetyl-D-fucosamine (D-FucNAc) residues and between about 2 to about 25 N-acetyl-D-quinovosamine (D-QuiNAc) in every 100 saccharide repeat units of the polysaccharide.

140. A S. pneumoniae serotype 12F glycoconjugate comprising a serotype 12F capsular polysaccharide comprising between about 2 to about 22.5 N-acetyl-D-fucosamine (D-FucNAc) residues and between about 2 to about 22.5 N-acetyl-D-quinovosamine (D-QuiNAc) in every 100 saccharide repeat units of the polysaccharide.

141. A S. pneumoniae serotype 12F glycoconjugate comprising a serotype 12F capsular polysaccharide comprising between about 2 to about 15 N-acetyl-D-fucosamine (D-FucNAc) residues and between about 2 to about 15 N-acetyl-D-quinovosamine (D-QuiNAc) in every 100 saccharide repeat units of the polysaccharide.

142. A S. pneumoniae serotype 12F glycoconjugate comprising a serotype 12F capsular polysaccharide comprising between about 2 to about 12.5 N-acetyl-D-fucosamine (D-FucNAc) residues and between about 2 to about 12.5 N-acetyl-D-quinovosamine (D-QuiNAc) in every 100 saccharide repeat units of the polysaccharide.

143. A S. pneumoniae serotype 12F glycoconjugate comprising a serotype 12F capsular polysaccharide comprising between about 3 to about 25 N-acetyl-D-fucosamine (D-FucNAc) residues and between about 3 to about 25 N-acetyl-D-quinovosamine (D-QuiNAc) in every 100 saccharide repeat units of the polysaccharide.

144. A S. pneumoniae serotype 12F glycoconjugate comprising a serotype 12F capsular polysaccharide comprising between about 3 to about 22.5 N-acetyl-D-fucosamine (D-FucNAc) residues and between about 3 to about 22.5 N-acetyl-D-quinovosamine (D-QuiNAc) in every 100 saccharide repeat units of the polysaccharide.

145. A S. pneumoniae serotype 12F glycoconjugate comprising a serotype 12F capsular polysaccharide comprising between about 3 to about 15 N-acetyl-D-fucosamine (D-FucNAc) residues and between about 3 to about 15 N-acetyl-D-quinovosamine (D-QuiNAc) in every 100 saccharide repeat units of the polysaccharide.

146. A S. pneumoniae serotype 12F glycoconjugate comprising a serotype 12F capsular polysaccharide comprising between about 3 to about 12.5 N-acetyl-D-fucosamine (D-FucNAc) residues and between about 3 to about 12.5 N-acetyl-D-quinovosamine (D-QuiNAc) in every 100 saccharide repeat units of the polysaccharide.

147. A S. pneumoniae serotype 12F glycoconjugate comprising a serotype 12F capsular polysaccharide comprising between about 4 to about 25 N-acetyl-D-fucosamine (D-FucNAc) residues and between about 4 to about 25 N-acetyl-D-quinovosamine (D-QuiNAc) in every 100 saccharide repeat units of the polysaccharide.

148. A S. pneumoniae serotype 12F glycoconjugate comprising a serotype 12F capsular polysaccharide comprising between about 4 to about 22.5 N-acetyl-D-fucosamine (D-FucNAc) residues and between about 4 to about 22.5 N-acetyl-D-quinovosamine (D-QuiNAc) in every 100 saccharide repeat units of the polysaccharide.

149. A S. pneumoniae serotype 12F glycoconjugate comprising a serotype 12F capsular polysaccharide comprising between about 4 to about 15 N-acetyl-D-fucosamine (D-FucNAc) residues and between about 4 to about 15 N-acetyl-D-quinovosamine (D-QuiNAc) in every 100 saccharide repeat units of the polysaccharide.

150. A S. pneumoniae serotype 12F glycoconjugate comprising a serotype 12F capsular polysaccharide comprising between about 4 to about 12.5 N-acetyl-D-fucosamine (D-FucNAc) residues and between about 4 to about 12.5 N-acetyl-D-quinovosamine (D-QuiNAc) in every 100 saccharide repeat units of the polysaccharide.

151. A S. pneumoniae serotype 12F glycoconjugate comprising a serotype 12F capsular polysaccharide comprising between about 5 to about 25 N-acetyl-D-fucosamine (D-FucNAc) residues and between about 5 to about 25 N-acetyl-D-quinovosamine (D-QuiNAc) in every 100 saccharide repeat units of the polysaccharide.

152. A S. pneumoniae serotype 12F glycoconjugate comprising a serotype 12F capsular polysaccharide comprising between about 5 to about 22.5 N-acetyl-D-fucosamine (D-FucNAc) residues and between about 5 to about 22.5 N-acetyl-D-quinovosamine (D-QuiNAc) in every 100 saccharide repeat units of the polysaccharide.

153. A S. pneumoniae serotype 12F glycoconjugate comprising a serotype 12F capsular polysaccharide comprising between about 5 to about 15 N-acetyl-D-fucosamine (D-FucNAc) residues and between about 5 to about 15 N-acetyl-D-quinovosamine (D-QuiNAc) in every 100 saccharide repeat units of the polysaccharide.

154. A S. pneumoniae serotype 12F glycoconjugate comprising a serotype 12F capsular polysaccharide comprising between about 5 to about 12.5 N-acetyl-D-fucosamine (D-FucNAc) residues and between about 5 to about 12.5 N-acetyl-D-quinovosamine (D-QuiNAc) in every 100 saccharide repeat units of the polysaccharide.

155. A S. pneumoniae serotype 12F glycoconjugate comprising a serotype 12F capsular polysaccharide comprising between about 7.5 to about 25 N-acetyl-D-fucosamine (D-FucNAc) residues and between about 7.5 to about 25 N-acetyl-D-quinovosamine (D-QuiNAc) in every 100 saccharide repeat units of the polysaccharide.

156. A S. pneumoniae serotype 12F glycoconjugate comprising a serotype 12F capsular polysaccharide comprising between about 7.5 to about 22.5 N-acetyl-D-fucosamine (D-FucNAc) residues and between about 7.5 to about 22.5 N-acetyl-D-quinovosamine (D-QuiNAc) in every 100 saccharide repeat units of the polysaccharide.

157. A S. pneumoniae serotype 12F glycoconjugate comprising a serotype 12F capsular polysaccharide comprising between about 7.5 to about 15 N-acetyl-D-fucosamine (D-FucNAc) residues and between about 7.5 to about 15 N-acetyl-D-quinovosamine (D-QuiNAc) in every 100 saccharide repeat units of the polysaccharide.

158. A S. pneumoniae serotype 12F glycoconjugate comprising a serotype 12F capsular polysaccharide comprising between about 7.5 to about 12.5 N-acetyl-D-fucosamine (D-FucNAc) residues and between about 7.5 to about 12.5 N-acetyl-D-quinovosamine (D-QuiNAc) in every 100 saccharide repeat units of the polysaccharide.

159. A S. pneumoniae serotype 12F glycoconjugate comprising a serotype 12F capsular polysaccharide comprising between about 10 to about 25 N-acetyl-D-fucosamine (D-FucNAc) residues and between about 10 to about 25 N-acetyl-D-quinovosamine (D-QuiNAc) in every 100 saccharide repeat units of the polysaccharide.

160. A S. pneumoniae serotype 12F glycoconjugate comprising a serotype 12F capsular polysaccharide comprising between about 10 to about 22.5 N-acetyl-D-fucosamine (D-FucNAc) residues and between about 10 to about 22.5 N-acetyl-D-quinovosamine (D-QuiNAc) in every 100 saccharide repeat units of the polysaccharide.

161. A S. pneumoniae serotype 12F glycoconjugate comprising a serotype 12F capsular polysaccharide comprising between about 10 to about 15 N-acetyl-D-fucosamine (D-FucNAc) residues and between about 10 to about 15 N-acetyl-D-quinovosamine (D-QuiNAc) in every 100 saccharide repeat units of the polysaccharide.

162. A S. pneumoniae serotype 12F glycoconjugate comprising a serotype 12F capsular polysaccharide comprising between about 0.05 to about 0.1 N-acetyl-D-fucosamine (D-FucNAc) residues and between about 0.05 to about 0.1 N-acetyl-D-quinovosamine (D-QuiNAc) in every 100 saccharide repeat units of the polysaccharide.

163. A S. pneumoniae serotype 12F glycoconjugate comprising a serotype 12F capsular polysaccharide comprising between about 0.05 to about 0.5 N-acetyl-D-fucosamine (D-FucNAc) residues and between about 0.05 to about 0.5 N-acetyl-D-quinovosamine (D-QuiNAc) in every 100 saccharide repeat units of the polysaccharide.

164. A S. pneumoniae serotype 12F glycoconjugate comprising a serotype 12F capsular polysaccharide comprising between about 0.05 to about 1 N-acetyl-D-fucosamine (D-FucNAc) residues and between about 0.05 to about 1 N-acetyl-D-quinovosamine (D-QuiNAc) in every 100 saccharide repeat units of the polysaccharide.

165. A S. pneumoniae serotype 12F glycoconjugate comprising a serotype 12F capsular polysaccharide comprising between about 0.1 to about 0.2 N-acetyl-D-fucosamine (D-FucNAc) residues and between about 0.1 to about 0.2 N-acetyl-D-quinovosamine (D-QuiNAc) in every 100 saccharide repeat units of the polysaccharide.

166. A S. pneumoniae serotype 12F glycoconjugate comprising a serotype 12F capsular polysaccharide comprising between about 0.1 to about 0.5 N-acetyl-D-fucosamine (D-FucNAc) residues and between about 0.1 to about 0.5 N-acetyl-D-quinovosamine (D-QuiNAc) in every 100 saccharide repeat units of the polysaccharide.

167. A S. pneumoniae serotype 12F glycoconjugate comprising a serotype 12F capsular polysaccharide comprising between about 0.1 to about 1 N-acetyl-D-fucosamine (D-FucNAc) residues and between about 0.1 to about 1 N-acetyl-D-quinovosamine (D-QuiNAc) in every 100 saccharide repeat units of the polysaccharide.

168. A S. pneumoniae serotype 12F glycoconjugate comprising a serotype 12F capsular polysaccharide comprising about 0.05 N-acetyl-D-fucosamine (D-FucNAc) residues and about 0.05 N-acetyl-D-quinovosamine (D-QuiNAc) in every 100 saccharide repeat units of the polysaccharide.

169. A S. pneumoniae serotype 12F glycoconjugate comprising a serotype 12F capsular polysaccharide comprising about 0.1 N-acetyl-D-fucosamine (D-FucNAc) residues and about 0.1 N-acetyl-D-quinovosamine (D-QuiNAc) in every 100 saccharide repeat units of the polysaccharide.

170. A S. pneumoniae serotype 12F glycoconjugate comprising a serotype 12F capsular polysaccharide comprising about 0.5 N-acetyl-D-fucosamine (D-FucNAc) residues and about 0.5 N-acetyl-D-quinovosamine (D-QuiNAc) in every 100 saccharide repeat units of the polysaccharide.

171. A S. pneumoniae serotype 12F glycoconjugate comprising a serotype 12F capsular polysaccharide comprising about 1 N-acetyl-D-fucosamine (D-FucNAc) residues and about 1 N-acetyl-D-quinovosamine (D-QuiNAc) in every 100 saccharide repeat units of the polysaccharide.

172. A S. pneumoniae serotype 12F glycoconjugate comprising a serotype 12F capsular polysaccharide comprising about 2 N-acetyl-D-fucosamine (D-FucNAc) residues and about 2 N-acetyl-D-quinovosamine (D-QuiNAc) in every 100 saccharide repeat units of the polysaccharide.

173. A S. pneumoniae serotype 12F glycoconjugate comprising a serotype 12F capsular polysaccharide comprising about 3 N-acetyl-D-fucosamine (D-FucNAc) residues and about 3 N-acetyl-D-quinovosamine (D-QuiNAc) in every 100 saccharide repeat units of the polysaccharide.

174. A S. pneumoniae serotype 12F glycoconjugate comprising a serotype 12F capsular polysaccharide comprising about 5 N-acetyl-D-fucosamine (D-FucNAc) residues and about 5 N-acetyl-D-quinovosamine (D-QuiNAc) in every 100 saccharide repeat units of the polysaccharide.

175. A S. pneumoniae serotype 12F glycoconjugate comprising a serotype 12F capsular polysaccharide comprising about 7 N-acetyl-D-fucosamine (D-FucNAc) residues and about 7 N-acetyl-D-quinovosamine (D-QuiNAc) in every 100 saccharide repeat units of the polysaccharide.

176. A S. pneumoniae serotype 12F glycoconjugate comprising a serotype 12F capsular polysaccharide comprising about 12 N-acetyl-D-fucosamine (D-FucNAc) residues and about 12 N-acetyl-D-quinovosamine (D-QuiNAc) in every 100 saccharide repeat units of the polysaccharide.

177. A S. pneumoniae serotype 12F glycoconjugate comprising a serotype 12F capsular polysaccharide comprising about 12.5 N-acetyl-D-fucosamine (D-FucNAc) residues and about 12.5 N-acetyl-D-quinovosamine (D-QuiNAc) in every 100 saccharide repeat units of the polysaccharide.

178. A S. pneumoniae serotype 12F glycoconjugate comprising a serotype 12F capsular polysaccharide comprising about 13 N-acetyl-D-fucosamine (D-FucNAc) residues and about 13 N-acetyl-D-quinovosamine (D-QuiNAc) in every 100 saccharide repeat units of the polysaccharide.

179. A S. pneumoniae serotype 12F glycoconjugate comprising a serotype 12F capsular polysaccharide comprising about 14 N-acetyl-D-fucosamine (D-FucNAc) residues and about 14 N-acetyl-D-quinovosamine (D-QuiNAc) in every 100 saccharide repeat units of the polysaccharide.

180. A S. pneumoniae serotype 12F glycoconjugate comprising a serotype 12F capsular polysaccharide comprising about 15 N-acetyl-D-fucosamine (D-FucNAc) residues and about 15 N-acetyl-D-quinovosamine (D-QuiNAc) in every 100 saccharide repeat units of the polysaccharide.

181. A S. pneumoniae serotype 12F glycoconjugate comprising a serotype 12F capsular polysaccharide comprising about 22.5 N-acetyl-D-fucosamine (D-FucNAc) residues and about 22.5 N-acetyl-D-quinovosamine (D-QuiNAc) in every 100 saccharide repeat units of the polysaccharide.

182. A S. pneumoniae serotype 12F glycoconjugate comprising a serotype 12F capsular polysaccharide comprising about 25 N-acetyl-D-fucosamine (D-FucNAc) residues and about 25 N-acetyl-D-quinovosamine (D-QuiNAc) in every 100 saccharide repeat units of the polysaccharide.

183. The glycoconjugate of any one of paragraphs 50-182 comprising a serotype 12F polysaccharide wherein the weight average molecular weight (Mw) of said polysaccharide before conjugation is between 50 kDa and 1,000 kDa.

184. The glycoconjugate of any one of paragraphs 50-182 comprising a serotype 12F polysaccharide wherein the weight average molecular weight (Mw) of said polysaccharide before conjugation is between 100 kDa and 600 kDa.

185. The glycoconjugate of any one of paragraphs 50-182 comprising a serotype 12F polysaccharide wherein the weight average molecular weight (Mw) of said polysaccharide before conjugation is between 150 kDa and 400 kDa.

186. The glycoconjugate of any one of paragraphs 50-185 having a weight average molecular weight (Mw) of between 250 kDa and 15,000 kDa.

187. The glycoconjugate of any one of paragraphs 50-185 having a weight average molecular weight (Mw) of between 500 kDa and 2,500 kDa.

188. The glycoconjugate of any one of paragraphs 50-185 having a weight average molecular weight (Mw) of between 1,000 kDa and 2,500 kDa.

189. The glycoconjugate of any one of paragraphs 50-188 wherein the degree of conjugation is between 2 and 15.

190. The glycoconjugate of any one of paragraphs 50-188 wherein the degree of conjugation is between 2 and 10.

191. The glycoconjugate of any one of paragraphs 50-188 wherein the degree of conjugation is between 2 and 6.

192. The glycoconjugate of any one of paragraphs 50-188 wherein the degree of conjugation is between 3 and 5.

193. The glycoconjugate of any one of paragraphs 50-188 wherein the degree of conjugation is between 4 and 10.

194. The glycoconjugate of any one of paragraphs 50-193 wherein the ratio of serotype 12F polysaccharide to carrier protein in the glycoconjugate (w/w) is between 0.5 and 3.0.

195. The glycoconjugate of any one of paragraphs 50-193 wherein the ratio of serotype 12F polysaccharide to carrier protein in the glycoconjugate (w/w) is between 0.5 and 2.0.

196. The glycoconjugate of any one of paragraphs 50-193 wherein the ratio of serotype 12F polysaccharide to carrier protein in the glycoconjugate (w/w) is between 0.5 and 1.5.

197. The glycoconjugate of any one of paragraphs 50-193 wherein the ratio of serotype 12F polysaccharide to carrier protein in the glycoconjugate (w/w) is between 0.8 and 1.2.

198. The glycoconjugate of any one of paragraphs 50-193 wherein the ratio of serotype 12F polysaccharide to carrier protein in the glycoconjugate (w/w) is between 0.5 and 1.0.

199. The glycoconjugate of any one of paragraphs 50-193 wherein the ratio of serotype 12F polysaccharide to carrier protein in the glycoconjugate (w/w) is between 1.0 and 1.5.

200. The glycoconjugate of any one of paragraphs 50-193 wherein the ratio of serotype 12F polysaccharide to carrier protein in the glycoconjugate (w/w) is between 0.9 and 1.1.

201. The glycoconjugate of any one of paragraphs 50-200 comprising less than about 50% of free serotype 12F polysaccharide compared to the total amount of serotype 12F polysaccharide.

202. The glycoconjugate of any one of paragraphs 50-200 comprising less than about 25% of free serotype 12F polysaccharide compared to the total amount of serotype 12F polysaccharide.

203. The glycoconjugate of any one of paragraphs 50-200 comprising less than about 20% of free serotype 12F polysaccharide compared to the total amount of serotype 12F polysaccharide.

204. The glycoconjugate of any one of paragraphs 50-200 comprising less than about 15% of free serotype 12F polysaccharide compared to the total amount of serotype 12F polysaccharide.

205. The glycoconjugate of any one of paragraphs 50-204 wherein at least 30% of the serotype 12F glycoconjugate has a K_{d} below or equal to 0.3 in a CL-4B column.

206. The glycoconjugate of any one of paragraphs 50-204 wherein at least 60% of the serotype 12F glycoconjugate has a K_{d} below or equal to 0.3 in a CL-4B column.

207. The glycoconjugate of any one of paragraphs 50-204 wherein between 50% and 80% of the serotype 12F glycoconjugate has a K_{d} below or equal to 0.3 in a CL-4B column.

208. The glycoconjugate of any one of paragraphs 50-204 wherein between 65% and 80% of the serotype 12F glycoconjugate has a K_{d} below or equal to 0.3 in a CL-4B column.

209. The glycoconjugate of any one of paragraphs 50-208 wherein the carrier protein of the glycoconjugate is selected from the group consisting of TT (tetanus toxoid), DT (Diphtheria toxoid), DT mutants (such as CRM₁₉₇), and a C5a peptidase from Streptococcus (SCP).

210. The glycoconjugate of any one of paragraphs 50-208 wherein the carrier protein of the glycoconjugate is DT (Diphtheria toxoid).

211. The glycoconjugate of any one of paragraphs 50-208 wherein the carrier protein of the glycoconjugate is TT (tetanus toxoid).

212. The glycoconjugate of any one of paragraphs 50-208 wherein the carrier protein of the glycoconjugate is PD (H. influenzae protein D).

213. The glycoconjugate of any one of paragraphs 50-208 wherein the carrier protein of the glycoconjugate is CRM₁₉₇.

214. The glycoconjugate of paragraph 213 wherein the CRM₁₉₇ comprises 1 to 15 lysine residues out of 39 covalently linked to the saccharide.

215. The glycoconjugate of paragraph 213 wherein the CRM₁₉₇ comprises 1 to 20 lysine residues out of 39 covalently linked to the saccharide.

216. The glycoconjugate of any one of paragraphs 50-215 wherein said glycoconjugate is prepared using reductive amination.

217. The glycoconjugate of paragraph 216 wherein before oxidation, sizing of the polysaccharide to a target molecular weight (MW) range is performed.

218. The glycoconjugate of any one of paragraphs 50-217 wherein said glycoconjugate is prepared by a process comprising the step of: a) reacting a serotype 12F saccharide with a stable nitroxyl radical compound and an oxidant to produce an activated saccharide; and b) reacting the activated saccharide with a carrier protein.

219. The glycoconjugate of paragraph 218 wherein said stable nitroxyl radical compound is a molecule bearing a TEMPO or a PROXYL (2,2,5,5-tetramethyl-1-pyrrolidinyloxy) moiety.

220. The glycoconjugate of paragraph 218 wherein said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl.

221. The glycoconjugate of paragraph 218 wherein said stable nitroxyl radical compound is TEMPO.

222. The glycoconjugate of any one of paragraphs 218-221 wherein said oxidant is a molecule bearing a N-halo moiety.

223. The glycoconjugate of any one of paragraphs 218-221 wherein said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione.

224. The glycoconjugate of any one of paragraphs 218-221 wherein said oxidant is N-Chlorosuccinimide.

225. The glycoconjugate of paragraph 218 wherein said stable nitroxyl radical compound is 2,2,6,6-Tetramethyl-1-piperidinyloxy free radical (TEMPO) and said oxidant is N-Chlorosuccinimide (NCS).

226. The glycoconjugate of any one of paragraphs 218-225 wherein at the end of the reduction reaction, the unreacted aldehyde groups remaining in the conjugates are capped using a capping agent.

227. The glycoconjugate of paragraph 226 wherein said capping agent is sodium borohydride (NaBH₄).

228. The glycoconjugate of any one of paragraphs 50-217 wherein said glycoconjugate is prepared by a process comprising the step of: (a) reacting an isolated serotype 12F polysaccharide with an oxidizing agent; (b) compounding the activated polysaccharide of step (a) with a carrier protein; and (c) reacting the compounded activated polysaccharide and carrier protein with a reducing agent to form a glycoconjugate.

229. The glycoconjugate of any one of paragraphs 50-217 wherein said glycoconjugate is prepared by a process comprising the step of: (a) reacting an isolated serotype 12F polysaccharide with an oxidizing agent; (a′) quenching the oxidation reaction by addition of a quenching agent; (b) compounding the activated polysaccharide of step (a′) with a carrier protein; and (c) reacting the compounded activated polysaccharide and carrier protein with a reducing agent to form a glycoconjugate.

230. The glycoconjugate of any one of paragraphs 228-229 wherein said oxidizing agent is periodate.

231. The glycoconjugate of any one of paragraphs 228-230 wherein the degree of oxidation of the activated serotype 12F polysaccharide is between 2 and 30. 232. The glycoconjugate of any one of paragraphs 228-231 wherein at the end of the reduction reaction, the unreacted aldehyde groups remaining in the conjugates are capped using a capping agent.

233. The glycoconjugate of paragraph 232 wherein said capping agent is sodium borohydride (NaBH₄).

234. An immunogenic composition comprising the polysaccharide of any one of paragraphs 1-49.

235. An immunogenic composition comprising the glycoconjugate of any one of paragraphs 50-234.

236. The immunogenic composition of paragraph 235 comprising from 1 to 25 glycoconjugates from different serotypes of S. pneumoniae.

237. The immunogenic composition of paragraph 235 comprising 20 glycoconjugates from different serotypes of S. pneumoniae.

238. The immunogenic composition of paragraph 235 which is a 20-valent pneumococcal conjugate composition.

239. The immunogenic composition of paragraph 235 further comprising glycoconjugates from S. pneumoniae serotypes 4, 6B, 9V, 14, 18C, 19F and 23F.

240. The immunogenic composition of paragraph 239 further comprising glycoconjugates from S. pneumoniae serotypes 1, 5 and 7F.

241. The immunogenic composition of paragraph 240 further comprising a glycoconjugate from S. pneumoniae serotype 3.

242. The immunogenic composition of paragraph 241 further comprising glycoconjugates from S. pneumoniae serotypes 6A and 19A.

243. The immunogenic composition of paragraph 242 further comprising glycoconjugates from S. pneumoniae serotype 22F and 33F.

244. The immunogenic composition of paragraph 243 further comprising glycoconjugates from S. pneumoniae serotypes 8, 10A, 11A and 15B.

245. The immunogenic composition of paragraph 244 which is a 20-valent pneumococcal conjugate composition

246. The immunogenic composition of any one of paragraphs 235-245 wherein the glycoconjugate from S. pneumoniae serotype 12F is conjugated to CRM₁₉₇.

247. The immunogenic composition of any one of paragraphs 235-246 wherein the glycoconjugates are all individually conjugated to CRM₁₉₇.

248. The immunogenic compositions of any one of paragraphs 235-247 for use as a medicament.

249. The immunogenic compositions of any one of paragraphs 235-247 for use as a vaccine.

250. A method of detecting the presence of 4-keto-N-acetyl-quinovosamine residues in an isolated S. pneumoniae serotype 12F polysaccharide, said method comprising the step of: a) isolating an S. pneumoniae serotype 12F polysaccharide and b) detecting the presence of 4-keto-N-acetyl-quinovosamine residues in said polysaccharide.

251. The method of paragraph 250 wherein the presence of 4-keto-N-acetyl-quinovosamine residues is detected by NMR or Mass Spectrometry (MS).

252. The method of paragraph 250 wherein the presence of 4-keto-N-acetyl-quinovosamine residues is detected by NMR.

253. The method of paragraph 250 wherein the presence of 4-keto-N-acetyl-quinovosamine residues is detected by Mass Spectrometry (MS).

254. A method of determining the amount of 4-keto-N-acetyl-quinovosamine residues in an isolated S. pneumoniae serotype 12F polysaccharide, said method comprising the step of: a) isolating an S. pneumoniae serotype 12F polysaccharide and b) measuring the amount of 4-keto-N-acetyl-quinovosamine residues in said polysaccharide.

255. The method of paragraph 254 wherein the amount of 4-keto-N-acetyl-quinovosamine residues is determined by NMR.

256. The method of paragraph 254 wherein the amount of 4-keto-N-acetyl-quinovosamine residues is determined by Mass Spectrometry (MS).

257. A method of detecting the presence of N-acetyl-D-fucosamine (D-FucNAc) residues in a reduced serotype 12F polysaccharide, said method comprising the step of: a) reacting an isolated S. pneumoniae serotype 12F polysaccharide with a reducing agent and b) detecting the presence of N-acetyl-D-fucosamine (D-FucNAc) residues in said reduced polysaccharide.

258. The method of paragraph 257 wherein the presence of N-acetyl-D-fucosamine (D-FucNAc) residues is detected by NMR.

259. The method of paragraph 254 wherein the presence of N-acetyl-D-fucosamine (D-FucNAc) residues is detected by Mass Spectrometry (MS).

260. The method of any one of paragraphs 254-259 wherein said reducing agent is sodium borohydride (NaBH₄).

261. The method of any one of paragraphs 254-260 wherein said isolated S. pneumoniae serotype 12F polysaccharide has been previously treated with an oxidizing agent.

262. The method of paragraphs 261 wherein the oxidizing agent is any oxidizing agent which oxidizes a terminal hydroxyl group to an aldehyde.

263. The method of paragraphs 261 wherein the oxidizing agent is periodate.

264. The method of any one of paragraphs 254-260 wherein said isolated S. pneumoniae serotype 12F polysaccharide has been previously treated with a stable nitroxyl radical compound and an oxidant.

265. The method of paragraph 263 wherein, said stable nitroxyl radical compound is 2,2,6,6-Tetramethyl-1-piperidinyloxy free radical (TEMPO) and said oxidant is N-Chlorosuccinimide (NCS).

266. A method of detecting the presence of N-acetyl-D-quinovosamine (D-QuiNAc) residues in a reduced serotype 12F polysaccharide, said method comprising the step of: a) reacting an isolated S. pneumoniae serotype 12F polysaccharide with a reducing agent and b) detecting the presence of N-acetyl-D-quinovosamine (D-QuiNAc) residues in said reduced polysaccharide.

267. The method of paragraph 266 wherein the presence of N-acetyl-D-quinovosamine (D-QuiNAc) residues is detected by NMR.

268. The method of paragraph 266 wherein the presence of N-acetyl-D-quinovosamine (D-QuiNAc) residues is detected by Mass Spectrometry (MS).

269. The method of any one of paragraphs 266-268 wherein said reducing agent is sodium borohydride (NaBH₄).

270. The method of any one of paragraphs 266-269 wherein said isolated S. pneumoniae serotype 12F polysaccharide has been previously treated with an oxidizing agent.

271. The method of paragraphs 270 wherein the oxidizing agent is any oxidizing agent which oxidizes a terminal hydroxyl group to an aldehyde.

272. The method of paragraphs 270 wherein the oxidizing agent is periodate.

273. The method of any one of paragraphs 266-269 wherein said isolated S. pneumoniae serotype 12F polysaccharide has been previously treated with a stable nitroxyl radical compound and an oxidant.

274. The method of paragraph 273 wherein, said stable nitroxyl radical compound is 2,2,6,6-Tetramethyl-1-piperidinyloxy free radical (TEMPO) and said oxidant is N-Chlorosuccinimide (NCS).

275. A method of detecting the presence of N-acetyl-D-fucosamine (D-FucNAc) and N-acetyl-D-quinovosamine (D-QuiNAc) residues in a reduced serotype 12F polysaccharide, said method comprising the step of: a) reacting an isolated S. pneumoniae serotype 12F polysaccharide with a reducing agent and b) detecting the presence of N-acetyl-D-fucosamine (D-FucNAc) and N-acetyl-D-quinovosamine (D-QuiNAc) residues in said reduced polysaccharide.

276. The method of paragraph 275 wherein the presence of N-acetyl-D-fucosamine (D-FucNAc) and N-acetyl-D-quinovosamine (D-QuiNAc) residues is detected by NMR.

277. The method of paragraph 275 wherein the presence of N-acetyl-D-fucosamine (D-FucNAc) and N-acetyl-D-quinovosamine (D-QuiNAc) residues is detected by Mass Spectrometry (MS).

278. In an embodiment, said reducing agent is sodium borohydride (NaBH₄).

279. The method of any one of paragraphs 275-278 wherein said reducing agent is sodium borohydride (NaBH₄).

280. The method of any one of paragraphs 275-279 wherein said isolated S. pneumoniae serotype 12F polysaccharide has been previously treated with an oxidizing agent.

281. The method of paragraphs 280 wherein the oxidizing agent is any oxidizing agent which oxidizes a terminal hydroxyl group to an aldehyde.

282. The method of paragraphs 280 wherein the oxidizing agent is periodate.

283. The method of any one of paragraphs 275-269 wherein said isolated S. pneumoniae serotype 12F polysaccharide has been previously treated with a stable nitroxyl radical compound and an oxidant.

284. The method of paragraph 283 wherein, said stable nitroxyl radical compound is 2,2,6,6-Tetramethyl-1-piperidinyloxy free radical (TEMPO) and said oxidant is N-Chlorosuccinimide (NCS).

285. A method of detecting the presence of N-acetyl-D-fucosamine (D-FucNAc) and/or N-acetyl-D-quinovosamine (D-QuiNAc) residues in S. pneumoniae serotype 12F glycoconjugate, said method comprising the step of: a) preparing a S. pneumoniae serotype 12F glycoconjugate and b) detecting the presence of N-acetyl-D-fucosamine (D-FucNAc) and/or N-acetyl-D-quinovosamine (D-QuiNAc) residues in said glycoconjugate.

286. The method of paragraph 285 wherein the presence of N-acetyl-D-fucosamine (D-FucNAc) and/or N-acetyl-D-quinovosamine (D-QuiNAc) residues is detected by NMR.

287. The method of paragraph 285 wherein the presence of N-acetyl-D-fucosamine (D-FucNAc) and/or N-acetyl-D-quinovosamine (D-QuiNAc) residues is detected by Mass Spectrometry (MS).

288. A method of detecting the presence of N-acetyl-D-fucosamine (D-FucNAc) and N-acetyl-D-quinovosamine (D-QuiNAc) residues in S. pneumoniae serotype 12F glycoconjugate, said method comprising the step of: a) preparing a S. pneumoniae serotype 12F glycoconjugate and b) detecting the presence of N-acetyl-D-fucosamine (D-FucNAc) and N-acetyl-D-quinovosamine (D-QuiNAc) residues in said glycoconjugate.

289. The method of paragraph 288 wherein the presence of N-acetyl-D-fucosamine (D-FucNAc) and N-acetyl-D-quinovosamine (D-QuiNAc) residues is detected by NMR.

290. The method of paragraph 288 wherein the presence of N-acetyl-D-fucosamine (D-FucNAc) and N-acetyl-D-quinovosamine (D-QuiNAc) residues is detected by Mass Spectrometry (MS).

291. A method of detecting the presence of N-acetyl-D-fucosamine (D-FucNAc) residues in S. pneumoniae serotype 12F glycoconjugate, said method comprising the step of: a) preparing a S. pneumoniae serotype 12F glycoconjugate and b) detecting the presence of N-acetyl-D-fucosamine (D-FucNAc) residues in said glycoconjugate.

292. The method of paragraph 291 wherein the presence of N-acetyl-D-fucosamine (D-FucNAc) residues is detected by NMR.

293. The method of paragraph 291 wherein the presence of N-acetyl-D-fucosamine (D-FucNAc) residues is detected by Mass Spectrometry (MS).

294. A method of detecting the presence of N-acetyl-D-quinovosamine (D-QuiNAc) residues in S. pneumoniae serotype 12F glycoconjugate, said method comprising the step of: a) preparing a S. pneumoniae serotype 12F glycoconjugate and b) detecting the presence of N-acetyl-D-quinovosamine (D-QuiNAc) residues in said glycoconjugate.

295. The method of paragraph 294 wherein the presence of N-acetyl-D-quinovosamine (D-QuiNAc) residues is detected by NMR.

296. The method of paragraph 294 wherein the presence of N-acetyl-D-quinovosamine (D-QuiNAc) residues is detected by Mass Spectrometry (MS).

As used herein, the term “about” means within a statistically meaningful range of a value, such as a stated concentration range, time frame, molecular weight, temperature or pH.

Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLES

### Example 1: Serotype 12F Capsular Polysaccharide Contains 4-keto-N-acetyl-quinovosamine (4KQ)

According to Leontein et al., (Leontein et al. (1981) Can. J. Chem. 59: 2081-2085) the polysaccharide repeating unit of serotype 12F consists of a linear trisaccharide backbone (one N-acetylfucosamine (FucpNAc), one N-acetylgalactosamine (GalpNAc) and one N-acetylmannuronic acid (ManpNAcA)) with two branches: a pendant α-galactopyranose (Galp) linked at C3 of FucpNAc and an α-Glcp-(1→2)-α-Glcp disaccharide branch linked at C3 of ManpNAcA.

The Pneumococcal polysaccharide 12F was studied by 2D NMR and Mass Spectrometry to characterize the polymer repeat unit. It has been surprisingly found that the serotype 12F polysaccharide actually contains partial substitution of N-acetyl-galactosamine by 4-keto-N-acetyl-quinovosamine (also referred as 2-acetamido-2,6-dideoxy-D-xylo-4-hexulose and Sug in the present document). This keto sugar variant (4KQ) has been identified that replaced the GalNAc residue at a statistical average of ˜20-25 mol % among 12F repeat units in a first strain.

Methods

NMR. For NMR analysis, samples were typically treated with tip sonication, and 12F polysaccharide dissolved in aqueous solvent was tip sonicated for up to 90 minutes over an ice bath. Samples were filtered with a 0.22-micron filter to remove tip particles, and in some cases were size separated using spin columns of fixed MWCO pore size. Sonicated samples with or without size separation were dialyzed using 3 kDa MWCO dialysis cassettes against water, frozen, lyophilized, and re-dissolved in D₂O with ˜0.55 mM TSP-d₄. Sample pH was between ˜6-7 and was modulated with small amounts of NaOD for high concentration samples due to acidity of carboxylic acid in backbone MannNAcA residue. The NMR data were collected on Bruker 5 mm DCH cryoprobe on a Bruker-Biospin AVANCE III NMR spectrometer operating at 500 MHz. The data processing was conducted using M-Nova v 12.0. The chemical shift reference was TSP-d₄ at 0 and −1.8 ppm ¹H and ¹³C, respectively. For 1D ¹H spectra processing, 0.5 Hz EM line broadening was used, and manual cubic spline baseline correction was applied. 1D ¹³C spectra were collected using power gating with 0.5 s. inter-scan recycle delay. 2D analyses included ¹H—¹H COSY, ¹H—¹H NOESY, ¹H—¹³C HSQC, ¹H—¹³C HMBC, ¹H—¹³C HSQC-TOCSY.

LC-MS. LC-MS and LC-MS/MS data were collected in a positive ionization mode on a Thermo Orbitrap Fusion Lumos Tribrid mass spectrometer equipped with an Agilent 1260 HPLC. Samples were injected and separated on a Waters hydrophilic interaction (HILIC) BEH spherical hybrid column. Mobile phase (MP) A is water with 0.1% formic acid (FA) and MPB is acetonitrile (ACN) with 0.1% FA. The elution gradient was delivered at 200 μL/min; 10-95% MPA in 35 min, returned to 10% MPA in 1 min and equilibrated for 15 min. The RF lens voltages were increased to 70 V to induce in-source fragmentation of polysaccharide. The MS/MS data was acquired using high-energy collision dissociation (HCD).

SEC/MS. SEC/MS data from the partial acid hydrolysis experiments was collected on a Thermo Orbitrap Elite mass spectrometer equipped with an Agilent 1260 HPLC. Samples were injected and separated on a BEH200 SEC column. Mobile phase (MP) A is water with 0.05% trifluoracetic acid (TFA) and MPB is acetonitrile (ACN) with 0.05% TFA. The isocratic gradient was delivered at 200 μL/min: 80% MPA A and 20% MPB.

Partial Acid Hydrolysis. Native 12F polysaccharide at 6.3 mg/ml in water was treated with TFA. To a HPLC vial, 100 μl of the 12F sample and 2 μl of neat TFA was added. The mixture was vortexed and put on hot plate set at 60° C. for 100 min, then 70° C. for 120 min, then 80° C. for 60 min. The sample was analyzed by SEC/MS on a Thermo Orbitrap Elite mass spectrometer.

Results

NMR data is consistent with a single heterologous serotype 12F polysaccharide with two distinct repeat unit populations. The main repeat unit population (˜75-80 mol %) is consistent with the component sugars and organization published by Leontein et al., (Leontein et al. (1981) Can. J. Chem. 59: 2081-2085). The secondary repeat unit population (˜20-25 mol %) is characterized by replacement of backbone GalNAc residue with Sug residue (2-acetamido-2,6 dideoxy-D-xylo-hexos-4-ulose), which is predominantly in the hydrate form in bulk aqueous solvent under typical temperature and pH conditions assayed (i.e. 75° C. and pH ˜6-7). FIGS. 1A, 1B and 1C illustrate the two repeat unit populations (without GalNAc replacement by Sug (FIG. 1A), and with GalNAc replacement by Sug (FIG. 1B)). There is no evidence for two distinct serotype 12F polysaccharides, and the NMR data best fits with a single serotype 12F polysaccharide with 20-25% average replacement of GalNAc with Sug (keto-sugar) (FIG. 1C). Site-specific resolved changes in serotype 12F repeat unit sugar residue ¹H and/or ¹³C chemical shift due to incorporation of Sug residue are illustrated by shaded circles in FIG. 1B.

Well resolved shifts due to Sug residue include position 6 CH3 ¹H signals of Sug, as well as modification of adjacent FucNAc position 6 CH3 ¹H signal due to Sug incorporation. A unique feature of the Sug residue is a significantly shielded position 6 CH3 ¹³C signal at ˜12.36 ppm, with a corresponding proton chemical shift of 1.3 ppm. This is a stronger shielding than expected for this type of methyl carbon and is due to the adjacent hydrated ketone at position 4. The hydrated ketone is sp3, does not have a bound proton, and the ˜94 ppm sharp and resolved ¹³C resonance was therefore confirmed by HMBC correlation to be the position 4 hydrated ketone carbon of the Sug residue (FIG. 2). The position 4 sp3 hydrated ketone ¹³C resonance at ˜94 ppm represents a serotype 12F signal that is unique to the Sug residue. Under typical analytic conditions (aqueous solvent, pH ˜6-7, 75° C.), the Sug ketone is predominantly in the hydrate form, with a weak signal at ˜203 ppm attributed to Sug ketone at an approximate ratio of 9:1 (hydrate:ketone) at position 4 of Sug residue in serotype 12F polysaccharide (see FIG. 3).

Accurate mole ratio of sugar residues in serotype 12F polysaccharide has been established based on deconvolution of 1D ¹³C spectra. By this deconvolution approach, the ratio of serotype 12F polysaccharide residues has been found to be α-L-FucNAc:α-D-Gal:β-D-GalNAc:Sug:β-D-MannNAcA:α-D-Glc (1:1:0.75:0.25:1:2) as shown in FIG. 1.

The Sug residue component of serotype 12F polysaccharide is systematically present at ˜20-25 mol % in different lots of polysaccharide from the same strain (Strain 3 of Table 3 below) and is a product of fermentation.

The location of the Sug residue in the polysaccharide repeat unit is identified using 2D ¹H-¹H NOESY NMR spectra. The NMR NOESY data indicate that Sug replaces GalNAc in the repeat unit (see NOESY correlations at FIG. 4).

Mass spectrometry was also used to help further elucidate the structure of this unique signal in the NMR. A mass spectrometry experiment was designed to produce the pseudo-molecular ion for a polysaccharide repeat unit (RU). By conducting MS/MS experiments on the RU pseudo-molecular ion at m/z 1094.3892 of the 12F polysaccharide, the total sequence of the repeat unit could be obtained.

FIG. 5 is an example of the repeat unit (RU) sequencing that can be accomplished with mass spectrometry. In this figure, all of the sugar rings are detected and identified. FIG. 5 is labeled with a letter from A to F representing the 5 sugar rings in the 12F RU. As shown in FIG. 5, the linear portion of the repeat unit makes up rings ABC. Side-chain sugars are placed on top of the sugar where they are connected.

FIG. 5 shows the structure of the 12F polysaccharide as an example of how the mass spectrometry data will be interpreted. The rings are labeled A through F and a short hand configuration of the order of the rings is also shown. Table 1 provides the molecular masses.

TABLE 1

| Symbol | Monosacarride | Chemical Formula | Mass (Da) |
|---|---|---|---|
| A | N-acetylgalactosamine | C8O5NH13 | 203.07937 |
| B | N-acetyl mannuronic acid | C8H11NO6 | 217.05864 |
| C | hexose | C6O5H10 | 162.05282 |
| D | hexose | C6O5H10 | 162.05282 |
| E | N-acetyl fucose | C8H13NO4 | 187.08446 |
| F | hexose | C6O5H10 | 162.05282 |

In an effort to determine if the 12F keto-sugar variant was a unique polysaccharide present in the serotype 12F polysaccharide or if the keto-sugar was part of the 12F polysaccharide substituting for GalNac ˜20% of the time, several experiments were carried out. The first was to see if two different polysaccharides could be separated using a HILIC column. Only one polysaccharide species was found that contained both the GalNAc and keto-sugar.

The next experiment involved looking for a dimer or trimer with all keto-sugars in the polysaccharide, which would provide evidence of two different polysaccharides.

12F polysaccharide partially hydrolysed was analized. The partial acid hydrolysis cleaves the glycosidic bond to produce species of various residue length. 4KQ is minus 18 Da compared to N-acetylgalactosamine and the dimer and trimer would produce hydrolysis products which are 18 Da lower in case only one polysaccharide contains the keto-sugar. In case of a second species (if there is a chain where the keto sugar has replaced all the of N-acetylgalactosamine), then the dimer and trimer would produce hydrolysis products which are 36 Da and 54 Da lower, respectively.

The species detected are consistent with the incorporation of about 1 out of every 4 RU has a keto-sugar. This data indicates that every polysaccharide chain of 12F contains the keto-sugar and there is not two keto-sugars in adjacent repeat units. There is no evidence of a chain where every N-acetylgalactosamine has been replaced by a keto-sugar.

These data support the fact that 12F is one polysaccharide that contains the keto-sugar 20-25% of the time in the repeat unit.

Collectively, the data support that serotype 12F polysaccharide is comprised of single heterologous polysaccharide chains with keto-sugar replacing N-Acetylgalactosamine in the repeat unit backbone at a statistical average of 20-25% (FIG. 1). There is no evidence of a chain where every N-acetylgalactosamine has been replaced by a keto sugar.

### Example 2: Structure of Reduced Serotype 12F Capsular Polysaccharide

Methods

Reduced 12F polysaccharide. Reduced 12F polysaccharide was produced as follows: 150 mg of hydrolysed 12F in 60 mL of water (2.5 mg/mL) at pH 7.0 was mixed with 2 mL of a NaBH4 solution (˜807 mM in water) at 150 rpm overnight at 23° C., then dialyzed against water using 7 KDa MWCO dialysis cassette. For 1D ¹H spectra processing, 0.5 Hz EM line broadening was used, and manual cubic spline baseline correction was applied. 1D ¹³C spectra were collected using power gating with 0.5 s. inter-scan recycle delay. 2D analyses included ¹H—¹H COSY, ¹H—¹H NOESY, ¹H—¹³C HSQC, ¹H-¹³C HMBC, ¹H-¹³C HSQC-TOCSY.

LC-MS and LC-MS/MS. LC-MS and LC-MS/MS data from the reduction of 12F with NaBD₄ were collected in a positive ionization mode on a Thermo Orbitrap Q Exactive mass spectrometer equipped with an Agilent 1260 HPLC. Samples were injected and separated on a Waters hydrophilic interaction (HILIC) BEH spherical hybrid column. Mobile phase (MP) A is water with 0.1% TFA and MPB is acetonitrile (ACN) with 0.1% TFA. The elution gradient was delivered at 200 μL/min; 30-70% MPA in 35 min, returned to 30% MPA in 1 min and equilibrated for 14 min. The RF lens voltages were increased to 60 V to induce in-source fragmentation of polysaccharide. The MS/MS data was acquired using high-energy collision dissociation (HCD).

Reaction with NaBD₄: Dissolve 6.7 mg/ml NaBD₄ in water. To 0.27 ml of 12F polysaccharide (at about 3 mg/ml in water) 0.03 ml of NaBD₄ was added and incubated for 3 hours at room temperature. Then 1 μl of neat acetic acid was added to quench the reaction.

Results

The ketone/hydrate of Sug residue is sensitive to reduction using NaBH4. NaBH4 treated serotype 12F polysaccharide is characterized by specific changes in Sug residue involving deshielding of position 6 methyl carbon, loss of sp3 carbon at ˜94 ppm without directly bound proton, and emergence of two weaker novel spin systems consistent with D-FucNAc and D-QuiNAc (FIG. 6). With reduction, the main serotype 12F polysaccharide spin system is unchanged (FIG. 1A), and the same pattern of heterogeneity in residues adjacent to incorporation site is observed.

Treatment of serotype 12F polysaccharide with NaBH₄ specifically reduces the position 4 of Sug residue from a ketone/hydrate to an alcohol, and transform the residue Sug to a mixture of D-FucNAc and D-QuiNAc, characterized by position 4 hydroxyl at axial and equatorial orientations, respectively as illustrated in FIG. 6.

After reduction with NaBD4 the mass of the RU and fragment ions containing the keto sugar residue are shifted 3.0219 Da higher. This atypical mass shift from deuterium, is unnatural and provides unique ions that do not have interference from fragment ions that contain natural isotopes of ¹²C, ¹³C ¹H, ¹⁶O and ¹⁴N. This data supports the reduction of a ketone with NaBD4 (see Table 2 and FIG. 7).

TABLE 2
Mass to Ion Structure Correlations for RU of 12F after reduction with NaBD4.
All fragment ions containing the A^{D} residue are shifted by 3.0219 Da indicting reduction
(addition of two hydrogens) and incorporation of one deuterium on the keto sugar residue,
A^{D}. This is excellent evidence the N-acetylgalactosamine has been replaced by the keto
sugar.

| Residue (see | | | |
|---|---|---|---|
| FIG. 7) | Theoretical m/z | Observed m/z | Mass Error (ppm) |
| A^{D}BCDEF | 1079.3996 | 1079.3997 | 0.1 |
| BCDEF | 891.3088 | 891.3092 | 0.4 |
| A^{D}BCEF | 917.3468 | 917.3471 | 0.4 |
| A^{D}BCD | 730.2623 | 730.2622 | −0.1 |
| A^{D}BCE | 755.2939 | 755.2935 | −0.6 |
| BCDE | 729.2560 | 729.2563 | 0.4 |
| A^{D}BC | 568.2095 | 568.2091 | −0.7 |
| A^{D}BE | 593.2411 | 593.2406 | −0.9 |
| BCE | 567.2032 | 567.2035 | 0.5 |
| BCD | 542.1716 | 542.1715 | −0.1 |
| A^{D}EF | 538.2353 | 538.2353 | 0.0 |
| A^{D}CD | 513.2037 | 513.2034 | −0.5 |
| EF plus hex | 512.1974 | 512.1978 | 0.8 |
| A^{D}B | 406.1566 | 406.1561 | −1.4 |
| BC | 380.1187 | 380.1186 | −0.4 |
| BE | 405.1504 | 405.1501 | −0.7 |
| EF | 350.1446 | 350.1445 | −0.2 |
| CD | 325.1129 | 325.1129 | 0.0 |
| A^{D}E | 376.1825 | 376.1825 | 0.0 |
| A^{D}C | 351.1508 | 351.1499 | −2.7 |
| A^{D} | 189.0980 | 189.0981 | 0.5 |
| B | 218.0659 | 218.0660 | 0.4 |
| E | 188.0917 | 188.0918 | 0.4 |
| \hat{ }A^{D} | 207.1086 | 207.1086 | 0.1 |
| A^{D}* | 171.0874 | 171.0876 | 0.9 |

\hat{ }= addition of water
*= loss of water

### Example 3: Level of 4KQ Substitution Among Circulating Clinical 12F Isolates

Methods

Production, purification and analysis of 12F polysaccharides. Culture stocks as frozen cell suspensions were prepared by growing to late exponential phase in soy hydrolysate medium and freezing at −70° C. Production of 12F polysaccharide in fermentation medium was performed by first developing seed cultures from the frozen stocks in a soy-hydrolysate medium. The starter culture was then used to inoculate the same culture medium; the fermentation was performed in a stirred bioreactor at 36° C. The broth was then lysed by adding N-lauryl-sulfonate to 0.1% and subjected to purification procedures.

Purification of the polysaccharides was conducted as described in WO2020/170190.

Results

To determine if the 4KQ substitution was common among circulating clinical isolates, 17 confirmed 12F clinical isolates were analyzed. All 17 of the 12F clinical isolates had evidence of this 4KQ substitution ranging from 2.5 to 10.2% of the repeat units (Table 3).

Four culture strains and purified 12F pneumococcal polysaccharides accessible at the ATCC (the American Type Culture Collection (ATCC, Manassas, VA USA), ref. 196-X) have also been analysed (Table 3).

The 4-KQ substituent amount was quantitated by 2D-NMR (see example 1). The % keto substitution in Pn-12F is determined using the ratio of the intensities of methyl peak corresponding to 4KQ relative to FucNAc.

The level of 4KQ substitution that occurs in fermentation depends on the strain which is used (see Table 3).

TABLE 3

| Source culture collection | Source country | % 4KQ by NMR |
|---|---|---|
| Strain 1 | Unknown | 1.9 |
| Strain 2 | Unknown | 5.0 |
| Strain 3 | USA | 24.6 |
| Strain 4 | Germany | 4.7 |
| Strain 5 | Unknown | ~45^{a} |
| Purified 12F (ATCC | Unknown | 0.1 |
| ref. 196-X) | | |
| Clinical isolate #1 | UK | 5.6 |
| Clinical isolate #2 | Spain | 3.9 |
| Clinical isolate #3 | Israel | 4.7 |
| Clinical isolate #4 | Canada | 4.5 |
| Clinical isolate #5 | US | 7.0 |
| Clinical isolate #6 | France | 10.2 |
| Clinical isolate #7 | Canada | 10.2 |
| Clinical isolate #8 | Canada | 7.7 |
| Clinical isolate #9 | Canada | 7.6 |
| Clinical isolate #10 | Canada | 7.0 |
| Clinical isolate #11 | Canada | 6.1 |
| Clinical isolate #12 | Greece | 5.7 |
| Clinical isolate #13 | France | 4.1 |
| Clinical isolate #14 | Belgium | 4.2 |
| Clinical isolate #15 | Singapore | 2.5 |
| Clinical isolate #16 | USA | 5.6 |
| Clinical isolate #17 | USA | 5.8 |

^{a}The sample contained larger amount of C-polysaccharide contaminant (compared to the other samples) creating some interfering signals which likely expanded measurement uncertainty.

### Example 4: Immunogenicity of 4KQ-Containing 12F Polysaccharide Conjugates in OPAs Using Clinical Isolates with Different Levels of 4KQ

Methods

S pneumoniae Microcolony Opsonophagocytic Assays (OPAs). Opsonophagocytic Assays were performed as described (see e.g. WO2018/134693). The assays quantitatively assess functional anti-S. pneumoniae antibodies by measuring bacterial killing in reactions containing serially diluted test sera, baby rabbit complement, and differentiated effector cells (HL-60). The OPA titer is the reciprocal of the serum dilution resulting in 50% reduction in the number of bacterial colony forming units (CFUs) when compared to the control without serum (defined as the background CFU). The titer is interpolated from the two dilutions that encompass this 50% killing cut-off. Titers from multiple determinations per sample are reported as geometric mean titers (GMT).

Results

To determine if 4KQ content of capsular polysaccharide impacts the ability of vaccine induced antibodies to bind to and kill S pneumoniae serotype 12F isolates, immunogenicity of a multi-valent vaccine containing a 12F conjugate and a multi-valent vaccine containing plain 12F polysaccharide were assessed against a set of S pneumoniae 12F isolates with a range of 4KQ modification levels. OPA assays were conducted for six 12F isolates with a range of 4KQ modification levels, and titers were generated for a set of sera from subjects immunized with a multi-valent vaccine containing a 12F conjugate (n=41), a multi-valent vaccine containing plain 12F polysaccharide (n=26) or a multi-valent vaccine which did not contain 12F polysaccharide (as a negative control, n=28). The 12F polysaccharide included in the multi-valent vaccine containing plain 12F polysaccharide has very low 4KQ incorporation (˜0.2%), while the 12F polysaccharide used in the multi-valent vaccine containing a 12F conjugate contains a ˜25% 4KQ modification level.

As shown in FIG. 8, both the vaccine containing a 12F conjugate (12F conj.) and the vaccine containing plain 12F polysaccharide (12F plain) immune sera were able to elicit bacterial killing responses of isolates with 4KQ modification levels between 1.9% to 27.5%, with no statistically significant differences between titers. These data indicate that the vaccines elicited OPA titers that are similar across strains expressing low to high 4KQ modification levels.

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. An isolated polysaccharide with the following repeating unit: 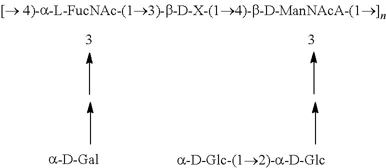
where n represents the number of repeating units and where X represents either N-acetylgalactosamine or 4-keto-N-acetyl-quinovosamine.

2. The isolated polysaccharide of claim 1 where said isolated polysaccharide comprises between about 99.9 to about 50 N-acetylgalactosamine residues and about 0.1 to about 50 4-keto-N-acetyl-quinovosamine residues in every 100 saccharide repeat units of the polysaccharide.

3. A S. pneumoniae serotype 12F glycoconjugate prepared by a process comprising the step of: a) reacting the isolated polysaccharide of claim 1 with an activating agent to produce an activated saccharide; and b) reacting the activated saccharide with a carrier protein.

4. A S. pneumoniae serotype 12F glycoconjugate comprising a serotype 12F capsular polysaccharide comprising between about 0.05 to about 25 N-acetyl-D-fucosamine (D-FucNAc) residues and/or between about 0.05 to about 25 N-acetyl-D-quinovosamine (D-QuiNAc) in every 100 saccharide repeat units of the polysaccharide.

5. The glycoconjugate of claim 3 comprising a serotype 12F polysaccharide wherein the weight average molecular weight (Mw) of said polysaccharide before conjugation is between 50 kDa and 1,000 kDa.

6. The glycoconjugate of claim 3 wherein the ratio of serotype 12F polysaccharide to carrier protein in the glycoconjugate (w/w) is between 0.5 and 3.0.

7. The glycoconjugate of claim 3 wherein the carrier protein of the glycoconjugate is TT (tetanus toxoid), DT (Diphtheria toxoid), DT mutants (such as CRM₁₉₇) or a C5a peptidase from Streptococcus (SCP).

8. The glycoconjugate of claim 3 wherein the carrier protein of the glycoconjugate is CRM₁₉₇.

9. The glycoconjugate of claim 8 wherein the CRM₁₉₇ comprises 1 to 15 lysine residues out of 39 covalently linked to the saccharide.

10. The glycoconjugate of claim 3 wherein said glycoconjugate is prepared using reductive amination.

11. The glycoconjugate of claim 3 wherein said glycoconjugate is prepared by a process comprising the step of: a) reacting a serotype 12F saccharide with a stable nitroxyl radical compound and an oxidant to produce an activated saccharide; and b) reacting the activated saccharide with a carrier protein.

12. The glycoconjugate of claim 3 wherein said glycoconjugate is prepared by a process comprising the step of: (a) reacting an isolated serotype 12F polysaccharide with an oxidizing agent; (b) compounding the activated polysaccharide of step (a) with a carrier protein; and (c) reacting the compounded activated polysaccharide and carrier protein with a reducing agent to form a glycoconjugate.

13. The glycoconjugate of claim 3 wherein said glycoconjugate is prepared by a process comprising the step of: (a) reacting an isolated serotype 12F polysaccharide with an oxidizing agent; (a′) quenching the oxidation reaction by addition of a quenching agent; (b) compounding the activated polysaccharide of step (a′) with a carrier protein; and (c) reacting the compounded activated polysaccharide and carrier protein with a reducing agent to form a glycoconjugate.

14. The glycoconjugate of claim 11 wherein the degree of oxidation of the activated serotype 12F polysaccharide is between 2 and 30.

15. An immunogenic composition comprising the polysaccharide of claim 1.

16. (canceled)

17. The immunogenic composition of claim 15 administered as a vaccine.

18. A method of detecting the presence of:
4-keto-N-acetyl-quinovosamine residues in an isolated S. pneumoniae serotype 12F polysaccharide, said method comprising the step of: a) isolating an S. pneumoniae serotype 12F polysaccharide and b) detecting the presence of 4-keto-N-acetyl-quinovosamine residues in said polysaccharide;
N-acetyl-D-fucosamine (D-FucNAc) residues in a reduced serotype 12F polysaccharide, said method comprising the step of: a) reacting an isolated S. pneumoniae serotype 12F polysaccharide with a reducing agent and b) detecting the presence of N-acetyl-D-fucosamine (D-FucNAc) residues in said reduced polysaccharide;
N-acetyl-D-quinovosamine (D-QuiNAc) residues in a reduced serotype 12F polysaccharide, said method comprising the step of: a) reacting an isolated S. pneumoniae serotype 12F polysaccharide with a reducing agent and b) detecting the presence of N-acetyl-D-quinovosamine (D-QuiNAc) residues in said reduced polysaccharide;
N-acetyl-D-fucosamine (D-FucNAc) and N-acetyl-D-quinovosamine (D-QuiNAc) residues in a reduced serotype 12F polysaccharide, said method comprising the step of: a) reacting an isolated S. pneumoniae serotype 12F polysaccharide with a reducing agent and b) detecting the presence of N-acetyl-D-fucosamine (D-FucNAc) and N-acetyl-D-quinovosamine (D-QuiNAc) residues in said reduced polysaccharide;
N-acetyl-D-fucosamine (D-FucNAc) and/or N-acetyl-D-quinovosamine (D-QuiNAc) residues in S. pneumoniae serotype 12F glycoconjugate, said method comprising the step of: a) preparing a S. pneumoniae serotype 12F glycoconjugate and b) detecting the presence of N-acetyl-D-fucosamine (D-FucNAc) and/or N-acetyl-D-quinovosamine (D-QuiNAc) residues in said glycoconjugate;
N-acetyl-D-fucosamine (D-FucNAc) residues in S. pneumoniae serotype 12F glycoconjugate, said method comprising the step of: a) preparing a S. pneumoniae serotype 12F glycoconjugate and b) detecting the presence of N-acetyl-D-fucosamine (D-FucNAc) residues in said glycoconjugate; and/or
N-acetyl-D-quinovosamine (D-QuiNAc) residues in S. pneumoniae serotype 12F glycoconjugate, said method comprising the step of: a) preparing a S. pneumoniae serotype 12F glycoconjugate and b) detecting the presence of N-acetyl-D-quinovosamine (D-QuiNAc) residues in said glycoconjugate.

19. A method of determining the amount of 4-keto-N-acetyl-quinovosamine residues in an isolated S. pneumoniae serotype 12F polysaccharide, said method comprising the step of: a) isolating an S. pneumoniae serotype 12F polysaccharide and b) measuring the amount of 4-keto-N-acetyl-quinovosamine residues in said polysaccharide.

20.-25. (canceled)

26. The isolated polysaccharide of claim 1 where said isolated polysaccharide comprises about 75 N-acetylgalactosamine residues and about 25 4-keto-N-acetyl-quinovosamine residues in every 100 saccharide repeat units of the polysaccharide.

27. An immunogenic composition comprising the glycoconjugate of claim 3.

28. The immunogenic composition of claim 27 administered as a vaccine.

29. The glycoconjugate of claim 4 comprising a serotype 12F capsular polysaccharide comprising between about 10 to about 15 N-acetyl-D-fucosamine (D-FucNAc) residues and between about 10 to about 15 N-acetyl-D-quinovosamine (D-QuiNAc) in every 100 saccharide repeat units of the polysaccharide.
